(19) United States
(12) Patent Application Publication
(10) Pub. No.: US 2026/0226479 A1
(43) Pub. Date: Aug. 6, 2026

(54) Methods for Inducing Modifications of Target Endogenous Nucleic Acid Sequences in Non-Human Embryos

(71) Applicant: TOOLGEN INCORPORATED, Seoul (KR)

(72) Inventors: Jin-Soo Kim, Seoul (KR); Seung Woo Cho, Seoul (KR); Sojung Kim, Seoul (KR)

(73) Assignee: TOOLGEN INCORPORATED, Seoul (KR)

(21) Appl. No.: 19/460,419

(22) Filed: Jan. 27, 2026

Related U.S. Application Data
(63) Continuation of application No. 19/027,181, filed on Jan. 17, 2025, now Pat. No. 12,612,634.
Application No. 19/027,181 is a continuation of application No. 18/932,745, filed on Oct. 31, 2024, now Pat. No. 12,612,632.
Application No. 18/932,745 is a continuation of application No. 17/004,338, filed on Aug. 27, 2020.
Application No. 17/004,338 is a continuation of application No. 14/685,568, filed on Apr. 13, 2015, now Pat. No. 10,851,380.
Application No. 14/685,568 is a continuation of application No. PCT/KR2013/009488, filed on Oct. 23, 2013.
(60) Provisional application No. 61/717,324, filed on Oct. 23, 2012.
(60) Provisional application No. 61/837,481, filed on Jun. 20, 2013.

(51) Int. Cl. C12N 15/52; C12N 9/16; C12N 9/22; C12N 15/10; C12N 15/11; C12N 15/63; C12N 15/82; C12N 15/85; C12N 15/90

(52) U.S. Cl. CPC C12N 15/52; C12N 9/16; C12N 9/22; C12N 15/102; C12N 15/111; C12N 15/63; C12N 15/8216; C12N 15/85; C12N 15/907; C12Y 301/21; C12N 2310/10; C12N 2310/20; C12N 2310/531

(57) ABSTRACT

The present disclosure relates to methods for producing a genome modified non-human embryo by inducing modifications at target endogenous nucleic acid sequences. For example, methods disclosed herein may comprise providing a Cas9/sgRNA complex, comprising a Cas9 protein and a single-guide RNA (sgRNA) in a cell-free buffer. Methods may further comprise introducing the Cas9/sgRNA complex into a non-human embryo, wherein the Cas9/sgRNA complex induces a modification at a target endogenous nucleic acid of the non-human embryo to provide for a genome modified embryo. Methods may further comprise transferring the genome modified embryo into a foster mother and allowing the foster mother to produce a F0 animal having the modification at the target endogenous nucleic acid.

### CROSS REFERENCE TO RELATED APPLICATIONS

The present application is a continuation application of U.S. application Ser. No. 19/027,181 filed Jan. 17, 2025, which is a continuation of U.S. application Ser. No. 18/932,745 filed Oct. 31, 2024, which is a continuation application of U.S. application Ser. No. 17/004,338 filed Aug. 27, 2020, which is a continuation application of U.S. application Ser. No. 14/685,568 filed Apr. 13, 2015, which is a continuation of PCT/KR2013/009488 filed Oct. 23, 2013, which claims priority to U.S. Provisional Application No. 61/837,481 filed on Jun. 20, 2013, U.S. Provisional Application No. 61/803,599 filed Mar. 20, 2013, and U.S. Provisional Application No. 61/717,324 filed Oct. 23, 2012, the entire contents of each aforementioned application are incorporated herein by reference.

### SEQUENCE LISTING

The instant application contains a Sequence Listing which has been submitted electronically in XML file format and is hereby incorporated by reference in its entirety. Said XML copy, created Jan. 23, 2026, is named 00375 SL.xml and is 495,147 bytes in size.

### TECHNICAL FIELD

The present invention relates to targeted genome editing in eukaryotic cells or organisms. More particularly, the present invention relates to a composition for cleaving a target DNA in eukaryotic cells or organisms comprising a guide RNA specific for the target DNA and Cas protein-encoding nucleic acid or Cas protein, and use thereof.

### BACKGROUND ART

CRISPRs (Clustered Regularly Interspaced Short Palindromic Repeats) are loci containing multiple short direct repeats that are found in the genomes of approximately 40% of sequenced bacteria and 90% of sequenced archaea. CRISPR functions as a prokaryotic immune system, in that it confers resistance to exogenous genetic elements such as plasmids and phages. The CRISPR system provides a form of acquired immunity. Short segments of foreign DNA, called spacers, are incorporated into the genome between CRISPR repeats, and serve as a memory of past exposures. CRISPR spacers are then used to recognize and silence exogenous genetic elements in a manner analogous to RNAi in eukaryotic organisms.

Cas9, an essential protein component in the Type II CRISPR/Cas system, forms an active endonuclease when complexed with two RNAs termed CRISPR RNA (crRNA) and trans-activating crRNA (tracrRNA), thereby slicing foreign genetic elements in invading phages or plasmids to protect the host cells. crRNA is transcribed from the CRISPR element in the host genome, which was previously captured from such foreign invaders. Recently, Jinek et al. (1) demonstrated that a single-chain chimeric RNA produced by fusing an essential portion of crRNA and tracrRNA could replace the two RNAs in the Cas9/RNA complex to form a functional endonuclease.

CRISPR/Cas systems offer an advantage to zinc finger and transcription activator-like effector DNA-binding proteins, as the site specificity in nucleotide binding CRISPR-Cas proteins is governed by a RNA molecule instead of the DNA-binding protein, which can be more challenging to design and synthesize.

However, until now, a genome editing method using the RNA-guided endonuclease (RGEN) based on CRISPR/Cas system has not been developed.

Meanwhile, Restriction fragment length polymorphism (RFLP) is one of the oldest, most convenient, and least expensive methods of genotyping that is still used widely in molecular biology and genetics but is often limited by the lack of appropriate sites recognized by restriction endonucleases.

Engineered nuclease-induced mutations are detected by various methods, which include mismatch-sensitive T7 endonuclease I (T7E1) or Surveyor nuclease assays, RFLP, capillary electrophoresis of fluorescent PCR products, Dideoxy sequencing, and deep sequencing. The T7E1 and Surveyor assays are widely used but are cumbersome. Furthermore, these enzymes tend to underestimate mutation frequencies because mutant sequences can form homoduplexes with each other and cannot distinguish homozygous bi-allelic mutant clones from wildtype cells. RFLP is free of these limitations and therefore is a method of choice. Indeed, RFLP was one of the first methods to detect engineered nuclease-mediated mutations in cells and animals. Unfortunately, however, RFLP is limited by the availability of appropriate restriction sites. It is possible that no restriction sites are available at the target site of interest.

### DISCLOSURE OF INVENTION

### Technical Problem

Until now, a genome editing and genotyping method using the RNA-guided endonuclease (RGEN) based on CRISPR/Cas system has not been developed.

Under these circumstances, the present inventors have made many efforts to develop a genome editing method based on CRISPR/Cas system and finally established a programmable RNA-guided endonuclease that cleave DNA in a targeted manner in eukaryotic cells and organisms.

In addition, the present inventors have made many efforts to develop a novel method of using RNA-guided endonucleases (RGENs) in RFLP analysis. They have used RGENs to genotype recurrent mutations found in cancer and those induced in cells and organisms by engineered nucleases including RGENs themselves, thereby completing the present invention.

### Solution to Problem

It is an object of the present invention to provide a composition for cleaving target DNA in eukaryotic cells or organisms comprising a guide RNA specific for target DNA or DNA that encodes the guide RNA, and Cas protein-encoding nucleic acid or Cas protein.

It is another object of the present invention to provide a composition for inducing targeted mutagenesis in eukaryotic cells or organisms, comprising a guide RNA specific for target DNA or DNA that encodes the guide RNA, and Cas protein-encoding nucleic acid or Cas protein.

It is still another object of the present invention to provide a kit for cleaving a target DNA in eukaryotic cells or organisms comprising a guide RNA specific for target DNA or DNA that encodes the guide RNA, and Cas protein-encoding nucleic acid or Cas protein.

It is still another object of the present invention to provide a kit for inducing targeted mutagenesis in eukaryotic cells or organisms comprising a guide RNA specific for target DNA or DNA that encodes the guide RNA, and Cas protein-encoding nucleic acid or Cas protein.

It is still another object of the present invention to provide a method for preparing a eukaryotic cell or organism comprising Cas protein and a guide RNA comprising a step of co-transfecting or serial-transfecting the eukaryotic cell or organism with a Cas protein-encoding nucleic acid or Cas protein, and a guide RNA or DNA that encodes the guide RNA.

It is still another object of the present invention to provide a eukaryotic cell or organism comprising a guide RNA specific for target DNA or DNA that encodes the guide RNA, and Cas protein-encoding nucleic acid or Cas protein.

It is still another object of the present invention to provide a method for cleaving a target DNA in eukaryotic cells or organisms comprising a step of transfecting the eukaryotic cells or organisms comprising a target DNA with a composition comprising a guide RNA specific for target DNA or DNA that encodes the guide RNA, and Cas protein-encoding nucleic acid or Cas protein.

It is still another object of the present invention to provide a method for inducing targeted mutagenesis in a eukaryotic cell or organism comprising a step of treating a eukaryotic cell or organism with a composition comprising a guide RNA specific for target DNA or DNA that encodes the guide RNA, and Cas protein-encoding nucleic acid or Cas protein.

It is still another object of the present invention to provide an embryo, a genome-modified animal, or genome-modified plant comprising a genome edited by a composition comprising a guide RNA specific for target DNA or DNA that encodes the guide RNA, and Cas protein-encoding nucleic acid or Cas protein.

It is still another object of the present invention to provide a method of preparing a genome-modified animal comprising a step of introducing the composition comprising a guide RNA specific for target DNA or DNA that encodes the guide RNA, and Cas protein-encoding nucleic acid or Cas protein into an embryo of an animal; and a step of transferring the embryo into a oviduct of pseudopregnant foster mother to produce a genome-modified animal.

It is still another object of the present invention to provide a composition for genotyping mutations or variations in an isolated biological sample, comprising a guide RNA specific for the target DNA sequence Cas protein.

It is still another object of the present invention to provide a method of using a RNA-guided endonuclease (RGEN) to genotype mutations induced by engineered nucleases in cells or naturally-occurring mutations or variations, wherein the RGEN comprises a guide RNA specific for target DNA and Cas protein.

It is still another object of the present invention to provide a kit for genotyping mutations induced by engineered nucleases in cells or naturally-occurring mutations or variations, comprising a RNA-guided endonuclease (RGEN), wherein the RGEN comprises a guide RNA specific for target DNA and Cas protein.

It is an object of the present invention to provide a composition for cleaving target DNA in eukaryotic cells or organisms comprising a guide RNA specific for target DNA or DNA that encodes the guide RNA, and Cas protein-encoding nucleic acid or Cas protein.

It is another object of the present invention to provide a composition for inducing targeted mutagenesis in eukaryotic cells or organisms, comprising a guide RNA specific for target DNA or DNA that encodes the guide RNA, and Cas protein-encoding nucleic acid or Cas protein.

It is still another object of the present invention to provide a kit for cleaving a target DNA in eukaryotic cells or organisms comprising a guide RNA specific for target DNA or DNA that encodes the guide RNA, and Cas protein-encoding nucleic acid or Cas protein.

It is still another object of the present invention to provide a kit for inducing targeted mutagenesis in eukaryotic cells or organisms comprising a guide RNA specific for target DNA or DNA that encodes the guide RNA, and Cas protein-encoding nucleic acid or Cas protein.

It is still another object of the present invention to provide a method for preparing a eukaryotic cell or organism comprising Cas protein and a guide RNA comprising a step of co-transfecting or serial-transfecting the eukaryotic cell or organism with a Cas protein-encoding nucleic acid or Cas protein, and a guide RNA or DNA that encodes the guide RNA.

It is still another object of the present invention to provide a eukaryotic cell or organism comprising a guide RNA specific for target DNA or DNA that encodes the guide RNA, and Cas protein-encoding nucleic acid or Cas protein.

It is still another object of the present invention to provide a method for cleaving a target DNA in eukaryotic cells or organisms comprising a step of transfecting the eukaryotic cells or organisms comprising a target DNA with a composition comprising a guide RNA specific for target DNA or DNA that encodes the guide RNA, and Cas protein-encoding nucleic acid or Cas protein.

It is still another object of the present invention to provide a method for inducing targeted mutagenesis in a eukaryotic cell or organism comprising a step of treating a eukaryotic cell or organism with a composition comprising a guide RNA specific for target DNA or DNA that encodes the guide RNA, and Cas protein-encoding nucleic acid or Cas protein.

It is still another object of the present invention to provide an embryo, a genome-modified animal, or genome-modified plant comprising a genome edited by a composition comprising a guide RNA specific for target DNA or DNA that encodes the guide RNA, and Cas protein-encoding nucleic acid or Cas protein.

It is still another object of the present invention to provide a method of preparing a genome-modified animal comprising a step of introducing the composition comprising a guide RNA specific for target DNA or DNA that encodes the guide RNA, and Cas protein-encoding nucleic acid or Cas protein into an embryo of an animal; and a step of transferring the embryo into a oviduct of pseudopregnant foster mother to produce a genome-modified animal.

It is still another object of the present invention to provide a composition for genotyping mutations or variations in an isolated biological sample, comprising a guide RNA specific for the target DNA sequence Cas protein.

It is still another object of the present invention to provide a composition for genotyping nucleic acid sequences in pathogenic microorganisms in an isolated biological sample, comprising a guide RNA specific for the target DNA sequence and Cas protein.

It is still another object of the present invention to provide a kit for genotyping mutations or variations in an isolated biological sample, comprising the composition, specifically comprising a RNA-guided endonuclease (RGEN), wherein the RGEN comprises a guide RNA specific for target DNA and Cas protein.

It is still another object of the present invention to provide a method of genotyping mutations or variations in an isolated biological sample, using the composition, specifically comprising a RNA-guided endonuclease (RGEN), wherein the RGEN comprises a guide RNA specific for target DNA and Cas protein.

### Advantageous Effects of Invention

The present composition for cleaving a target DNA or inducing a targeted mutagenesis in eukaryotic cells or organisms, comprising a guide RNA specific for the target DNA and Cas protein-encoding nucleic acid or Cas protein, the kit comprising the composition, and the method for inducing targeted mutagenesis provide a new convenient genome editing tools. In addition, because custom RGENs can be designed to target any DNA sequence, almost any single nucleotide polymorphism or small insertion/deletion (indel) can be analyzed via RGEN-mediated RFLP, therefore, the composition and method of the present invention may be used in detection and cleaving naturally-occurring variations and mutations.

### BRIEF DESCRIPTION OF DRAWINGS

FIGS. 1A and 1B show Cas9-catalyzed cleavage of plasmid DNA in vitro. FIG. 1A: Schematic representation of target DNA (SEQ ID NO: 112) and chimeric RNA sequences (SEQ ID NO: 113). Triangles indicate cleavage sites. The PAM sequence recognized by Cas9 is shown in bold. The sequences in the guide RNA (SEQ ID NO: 113) derived from crRNA and tracrRNA are shown in box and underlined, respectively. FIG. 1B: In vitro cleavage of plasmid DNA by Cas9. An intact circular plasmid or ApaLI-digested plasmid was incubated with Cas9 and guide RNA.

FIGS. 2A and 2B show Cas9-induced mutagenesis at an episomal target site. FIG. 2A: Schematic overview of cell-based assays using a RFP-GFP reporter. GFP is not expressed from this reporter because the GFP sequence is fused to the RFP sequence out-of-frame. The RFP-GFP fusion protein is expressed only when the target site between the two sequences is cleaved by a site-specific nuclease. FIG. 2B: Flow cytometry of cells transfected with Cas9. The percentage of cells that express the RFP-GFP fusion protein is indicated.

FIGS. 3A and 3B show RGEN-driven mutations at endogenous chromosomal sites. FIG. 3A: CCR5 locus. FIG. 3B: C4BPB locus. (Top) The T7E1 assay was used to detect RGEN-driven mutations. Arrows indicate the expected position of DNA bands cleaved by T7E1. Mutation frequencies (Indels (%)) were calculated by measuring the band intensities. (Bottom) DNA sequences of the wild-type (WT) CCR5 (SEQ ID NO: 114) and C4BPB (SEQ ID NO: 122) and mutant clones. DNA sequences of RGEN-induced mutations at the CCR5 locus: +1 (SEQ ID NO: 115), −13 (SEQ ID NO: 116), −14 (SEQ ID NO: 117), −18 (SEQ ID NO: 118), −19 (SEQ ID NO: 119), −24 (SEQ ID NO: 120), and −30 (SEQ ID NO: 121). DNA sequences of RGEN-induced mutations at the C4BPB locus: +1 (SEQ ID NO: 122), +2 (SEQ ID NO: 123), −30 (SEQ ID NO: 125), and −180 (SEQ ID NO: 126). The region of the target sequence complementary to the guide RNA is shown in box. The PAM sequence is shown in bold. Triangles indicate the cleavage site. Bases corresponding to microhomologies are underlined. The column on the right indicates the number of inserted or deleted bases.

FIGS. 4A, 4B, and 4C show that RGEN-driven off-target mutations are undetectable. FIG. 4A: On-target and potential off-target sequences. The human genome was searched in silico for potential off-target sites. Four sites were identified, ADCY5 (SEQ ID NO: 128), KCNJ6 (SEQ ID NO: 129), CNTNAP2 (SEQ ID NO: 130), and Chr. 5 N/A (SEQ ID NO: 131), each of which carries 3-base mismatches with the CCR5 on-target (SEQ ID NO: 127). Mismatched bases are underlined. FIG. 4B: The T7E1 assay was used to investigate whether these sites were mutated in cells transfected with the Cas9/RNA complex. No mutations were detected at these sites. N/A (not applicable), an intergenic site. FIG. 4C: Cas9 did not induce off-target-associated chromosomal deletions. The CCR5-specific RGEN and ZFN were expressed in human cells. PCR was used to detect the induction of the 15-kb chromosomal deletions in these cells.

FIGS. 5A, 5B, 5C, and 5D show RGEN-induced Foxn1 gene targeting in mice. FIG. 5A: A schematic diagram depicting target DNA (SEQ ID NO: 132) and a sgRNA specific to exon 2 of the mouse Foxn1 gene (SEQ ID NO: 133). PAM in exon 2 is shown in a box and the sequence in the sgRNA that is complementary to exon 2 is underlined. Triangles indicate cleavage sites.

FIG. 5B: Representative T7E1 assays demonstrating gene-targeting efficiencies of Cas9 mRNA plus Foxn1-specific sgRNA that were delivered via intra-cytoplasmic injection into one-cell stage mouse embryos. Numbers indicate independent founder mice generated from the highest dose. Arrows indicate bands cleaved by T7E1. FIG. 5C: DNA sequences of wild-type (WT) Foxn1 (SEQ ID NO: 134) and mutant alleles (SEQ ID NOs. 135-141) observed in three Foxn1 mutant founders identified in FIG. 5B. DNA sequences of mutant alleles in founder #108: −44 (SEQ ID NO: 135), −23 (SEQ ID NO: 136), −17 (SEQ ID NO: 137), and +1 (SEQ ID NO: 138). DNA sequences of mutant alleles in founder #111: +1 (SEQ ID NO: 138) and −11 (SEQ ID NO: 139). DNA sequences of mutant alleles in founder #114: −6 (SEQ ID NO: 140), −17 (SEQ ID NO: 137), and −8 (SEQ ID NO: 141). The number of occurrences is shown in parentheses. FIG. 5D: PCR genotyping of F1 progenies derived from crossing Foxn1 founder #108 and wild-type FVB/NTac. Note the segregation of the mutant alleles found in Foxn1 founder #108 in the progenies.

FIGS. 6A, 6B, and 6C show Foxn1 gene targeting in mouse embryos by intra-cytoplasmic injection of Cas9 mRNA and Foxn1-sgRNA. FIG. 6A: A representative result of a T7E1 assay monitoring the mutation rate after injecting the highest dose. Arrows indicate bands cleaved by T7E1. FIG. 6B: A summary of T7E1 assay results. Mutant fractions among in vitro cultivated embryos obtained after intra-cytoplasmic injection of the indicated RGEN doses are indicated. FIG. 6C: DNA sequences of wild-type (WT) Foxn1 (SEQ ID NO: 143) and Foxn1 mutant alleles (SEQ ID Nos. 144-152) identified from a subset of T7E1-positive mutant embryos. The DNA sequences of the mutant alleles are: Δ11 (SEQ ID NO: 144), Δ11+Δ17 (SEQ ID NO: 145) Δ57 (SEQ ID NO: 146), 017 (SEQ ID NO: 147), +1 (SEQ ID NO: 148), Δ12 (SEQ ID NO: 149, 072 (SEQ ID NO: 150), Δ25 (SEQ ID NO:151), Δ24 (SEQ ID NO: 152). The target sequence of the wild-type allele is denoted in box.

FIGS. 7A, 7B, and 7C show Foxn1 gene targeting in mouse embryos using the recombinant Cas9 protein: Foxn1-sgRNA complex. FIG. 7A and FIG. 7B are representative T7E1 assays results and their summaries. Embryos were cultivated in vitro after they underwent pronuclear (FIG. 7A) or intra-cytoplasmic injection (FIG. 7B). Underlined numbers indicate T7E1-positive mutant founder mice. FIG. 7C: DNA sequences of wild-type (WT) Foxn1 (SEQ ID NO: 153) and Foxn1 mutant alleles (SEQ ID NOs. 154-166) identified from the in vitro cultivated embryos that were obtained by the pronucleus injection of recombinant Cas9 protein: Foxn1-sgRNA complex at the highest dose. The target sequence of the wild-type allele is denoted in box. The DNA sequences of the mutant alleles are: Δ18 (SEQ ID NO: 154), Δ20 (SEQ ID NO: 155), Δ19 (SEQ ID NO: 156), Δ17 (SEQ ID NO: 157), All (SEQ ID NO: 158), Δ3+1 (SEQ ID NO: 159), Δ2 (SEQ ID NO: 160), +1, Embryo 1 (SEQ ID NO: 161), +1, Embryo 10 (SEQ ID NO: 162), Δ6 (SEQ ID NO: 163), Δ5 (SEQ ID NO: 164), Δ28 (SEQ ID NO: 165), and Δ126 (SEQ ID NO: 166).

FIGS. 8A, 8B, and 8C show Germ-line transmission of the mutant alleles found in Foxn1 mutant founder #12. FIG. 8A: wild type fPCR analysis. FIG. 8B: Foxn1 mutant founder #12 fPCR analysis. FIG. 8C: PCR genotyping of wild-type FVB/NTac, the founder mouse, and their F1 progenies.

FIGS. 9A and 9B show Genotypes of embryos generated by crossing Prkdc mutant founders. Prkdc mutant founders ♂25 and ♀15 were crossed and E13.5 embryos were isolated. FIG. 9A: fPCR analysis of wild-type, founder ♂25, and founder ♀15. Note that, due to the technical limitations of fPCR analysis, these results showed small differences from the precise sequences of the mutant alleles; e.g., from the sequence analysis, Δ269/Δ61/WT and Δ5+1/+7/+12/WT were identified in founders ♂25 and ♀15, respectively. FIG. 9B: Genotypes of the generated embryos.

FIGS. 10A, 10B, 10C, 10D, and 10E show Cas9 protein/sgRNA complex induced targeted mutation at CCR5 gene (FIGS. 10A-10C) and ABCC11 gene (FIGS. 10D-10E). FIG. 10A: Results of a T7E1 assay monitoring the mutation rate at CCR5 locus after introducing Cas9 protein and sgRNA or Cas9 protein and crRNA+tracrRNA into K562 cells. FIG. 10B: Results of a T7E1 assay using ⅕ scaled down doses of Cas9 protein and sgRNA. FIG. 10C: Wild-type (WT) CCR5 sequence (SEQ ID NO: 114) and Cas protein induced mutant sequences (SEQ ID NOs. 167-171 and 115) identified in CCR5 locus. The DNA sequences of the mutant sequences are: −4 (SEQ ID NO: 167), −4 (SEQ ID NO: 168), −7 (SEQ ID NO: 169), −1 (SEQ ID NO: 170), +1 (SEQ ID NO: 115), and −17, +1 (SEQ ID NO: 171). FIG. 10D: Results of a T7E1 assay monitoring the mutation rate at ABCC11 locus after introducing Cas9 protein and sgRNA into K562 cells. FIG. 10E: Wild-type (WT) ABCC11 sequence (SEQ ID NO: 172) and Cas9 protein induced mutant sequences (SEQ ID NOs. 173-176) identified in ABCC11 locus. The DNA sequences of the mutant sequences are: −6 (SEQ ID NO: 173), −3 (SEQ ID NO: 174), −29 (SEQ ID NO: 175), −20 (SEQ ID NO: 176), and −256 (TTCTC).

FIG. 11 shows recombinant Cas9 protein-induced mutations in Arabidopsis protoplasts.

FIG. 12 shows wild type BRI1 sequence (SEQ ID NO: 177) and recombinant Cas9 protein-induced mutant sequences (SEQ ID NOs. 178-181) in the Arabidopsis BRI1 gene. The DNA sequences of the mutant sequences are: −7 (SEQ ID NO: 178), −224 (SEQ ID NO: 179), −223 (SEQ ID NO: 180), and −223, +62 (SEQ ID NO: 181).

FIG. 13 shows T7E1 assay showing endogenous CCR5 gene disruption in 293 cells by treatment of Cas9-mal-9R4L and sgRNA/C9R4LC complex.

FIGS. 14A and 14B show mutation frequencies at on-target and off-target sites of RGENs reported in Fu et al. (2013). T7E1 assays analyzing genomic DNA from K562 cells (R) transfected serially with 20 μg of Cas9-encoding plasmid and with 60 μg and 120 μg of in vitro transcribed GX19 crRNA and tracrRNA, respectively (1×10⁶ cells), or (D) co-transfected with 1 μg of Cas9-encoding plasmid and 1 μg of GX₁₉ sgRNA expression plasmid (2×10⁵ cells). FIG. 14A: VEGFA site 1 on target sequence (SEQ ID NO: 182) and off target sequences, OT1-3 (SEQ ID NO: 183) and OT1-11 (SEQ ID NO: 184). VEGFA site 2 on target sequence (SEQ ID NO: 185) and off target sequences OT2-1 (SEQ ID NO: 186), OT2-9 (SEQ ID NO: 187) and OT2-24 (SEQ ID NO: 188). FIG. 14B: VEGFA site 3 on target sequence (SEQ ID NO: 189) and off target sequence OT3-18 (SEQ ID NO: 190) and EMX1 on target sequence (SEQ ID NO: 191) and off target sequence OT4-1 (SEQ ID NO: 192).

FIGS. 15A and 15B show comparison of guide RNA structure. Mutation frequencies of the RGENs reported in Fu et al. (2013) were measured at on-target and off-target sites using the T7E1 assay. K562 cells were co-transfected with the Cas9-encoding plasmid and the plasmid encoding GX19 sgRNA or GGX20 sgRNA. Off-target sites (OT1-3 etc.) are labeled as in Fu et al. (2013). FIG. 15A: VEGFA site 1 on target sequence (SEQ ID NO: 182) and off target sequences OT1-3 (SEQ ID NO: 183 and OT1-11 (SEQ ID NO: 184). VEGFA site 2 on target sequence (SEQ ID NO: 185) and off target sequences OT2-1 (SEQ ID NO: 186), OT2-9 (SEQ ID NO: 187), and OT2-24 (SEQ ID NO: 188). FIG. 15B: VEGFA site 3 on target sequence (SEQ ID NO: 189) and off target sequence OT3-18 (SEQ ID NO: 190) and EMX1 on target sequence (SEQ ID NO: 191) and off target sequence OT4-1 (SEQ ID NO: 192).

FIGS. 16A, 16B, 16C, and 16D show that in vitro DNA cleavage by Cas9 nickases. FIG. 16A: Schematic overview of the Cas9 nuclease and the paired Cas9 nickase. The PAM sequences and cleavage sites are shown in box. FIG. 16B: Target sites in the human AAVS1 locus. The position of each target site is shown in triangle. FIG. 16C: Schematic overview of DNA cleavage reactions. FAM dyes (shown in box) were linked to both 5′ ends of the DNA substrate. FIG. 16D: DSBs and SSBs analyzed using fluorescent capillary electrophoresis. Fluorescently-labeled DNA substrates were incubated with Cas9 nucleases or nickases before electrophoresis.

FIGS. 17A and 17B show comparison of Cas9 nuclease and nickase behavior. FIG. 17A: On-target mutation frequencies associated with Cas9 nucleases (WT), nickases (D10A), and paired nickases at the following target sequences of the AAVS1 locus: S1 (SEQ ID NO: 193, S2 (SEQ ID NO: 194), S3 (SEQ ID NO: 195), S4 (SEQ ID NO: 196), S5 (SEQ ID NO: 197), S6 (SEQ ID NO: 198), AS1 (SEQ ID NO: 199), AS2 (SEQ ID NO: 200), and AS3 (SEQ ID NO: 201). Paired nickases that would produce 5′ overhangs or 3′ overhangs are indicated. FIG. 17B: Analysis of off-target effects of Cas9 nucleases and paired nickases. A total of seven potential off-target sites (SEQ ID NOs. 202-208) for three sgRNAs were analyzed. The mutation frequency for the S2 on-target sequence (SEQ ID NO: 194) was compared to the off-target sequences, S2 Off-1 (SEQ ID NO: 202) and S2 Off-2 (SEQ ID NO: 203). The mutation frequency for the S3 on-target sequence (SEQ ID NO: 195) was compared to the off-target sequences, S3 Off-1 (SEQ ID NO: 204) and S3 Off-2 (SEQ ID NO: 205). The mutation frequency for the AS2 on-target sequence (SEQ ID NO: 198) was compared to the off-target sequences, AS2 Off-1 (SEQ ID NO: 206), AS2 Off-6 (SEQ ID NO: 207), and AS2 Off-9 (SEQ ID NO: 208).

FIGS. 18A, 18B, 18C, and 18D show paired Cas9 nickases tested at other endogenous human loci. The sgRNA target sites at the human CCR5 locus (FIG. 18A; SEQ ID NO: 209) and the BRCA2 locus (FIG. 18C; SEQ ID NO: 210). PAM sequences are indicated in a box. Genome editing activities at CCR5 (FIG. 18B) and BRCA2 (FIG. 18D) target sites were detected by the T7E1 assay. The repair of two nicks that would produce 5′ overhangs led to the formation of indels much more frequently than did those producing 3′ overhangs.

FIGS. 19A and 19B show that paired Cas9 nickases mediate homologous recombination. FIG. 19A: Strategy to detect homologous recombination. Donor DNA included an XbaI restriction enzyme site between two homology arms, whereas the endogenous target site lacked this site. A PCR assay was used to detect sequences that had undergone homologous recombination. To prevent amplification of contaminating donor DNA, primers specific to genomic DNA were used. FIG. 19B: Efficiency of homologous recombination. Only amplicons of a region in which homologous recombination had occurred could be digested with XbaI; the intensities of the cleavage bands were used to measure the efficiency of this method.

FIGS. 20A, 20B, 20C, and 20D show DNA splicing induced by paired Cas9 nickases. FIG. 20A: The target sites of paired nickases in the human AAVS1 locus. The distances between the AS2 site and each of the other sites are shown. Arrows indicate PCR primers. FIG. 20B: Genomic deletions detected using PCR. Asterisks indicate deletion-specific PCR products. FIG. 20C: DNA sequences of wild-type (WT) (SEQ ID NO: 211 and 332) and the following deletion-specific PCR products (SEQ ID Nos. 212-218) obtained using AS2 sgRNAs or deletion-specific PCR products (SEQ ID NOs. 219-224) using Li sgRNAs. Target site PAM sequences are shown in box and sgRNA-matching sequences are shown in capital letters. Intact sgRNA-matching sequences are underlined. FIG. 20D: A schematic model of paired Cas9 nickase-mediated chromosomal deletions. Newly-synthesized DNA strands are shown in box.

FIGS. 21A, 21B, and 21C show that paired Cas9 nickases do not induce translocations. FIG. 21A: Schematic overview of chromosomal translocations between the on-target and off-target sites. FIG. 21B: PCR amplification to detect chromosomal translocations. FIG. 21C: Translocations induced by Cas9 nucleases but not by the nickase pair.

FIGS. 22A and 22B show a conceptual diagram of the T7E1 and RFLP assays. FIG. 22A: Comparison of assay cleavage reactions in four possible scenarios after engineered nuclease treatment in a diploid cell: (A) wild type, (B) a monoallelic mutation, (C) different biallelic mutations (hetero), and (D) identical biallelic mutations (homo). Black lines represent PCR products derived from each allele; dashed and dotted boxes indicate insertion/deletion mutations generated by NHEJ. FIG. 22B: Expected results of T7E1 and RGEN digestion resolved by electrophoresis.

FIG. 23 shows in vitro cleavage assay of a linearized plasmid containing the C4BPB target site bearing indels. DNA sequences of individual plasmid substrates (upper panel): WT (SEQ ID NO: 104), I1 (SEQ ID NO: 225), I2 (SEQ ID NO: 226), I3 (SEQ ID NO: 227), D1 (SEQ ID NO: 228), D2 (SEQ ID NO: 229), and D3 (SEQ ID NO: 230). The PAM sequence is underlined. Inserted bases are shown in box. Arrows (bottom panel) indicate expected positions of DNA bands cleaved by the wild-type-specific RGEN after electrophoresis.

FIGS. 24A and 24B show genotyping of mutations induced by engineered nucleases in cells via RGEN-mediated RFLP. FIG. 24A: Genotype of C4BPB wild type (SEQ ID NO: 231) and the following mutant K562 cell clones: +3 (SEQ ID NO: 232, −12 (SEQ ID NO: 233), −9 (SEQ ID NO: 234), −8 (SEQ ID NO: 235), −36 (SEQ ID NO: 236), +1 (SEQ ID NO: 237), +1 (SEQ ID NO: 238), +67 (SEQ ID NO: 239), −7, +1 (SEQ ID NO: 240), −94 (SEQ ID NO: 241). FIG. 24B: Comparison of the mismatch-sensitive T7E1 assay with RGEN-mediated RFLP analysis. Black arrows indicate the cleavage product by treatment of T7E1 enzyme or RGENs.

FIGS. 25A, 25B, and 25C show genotyping of RGEN-induced mutations via the RGEN-RFLP technique. FIG. 25A: Analysis of C4BPB-disrupted clones using RGEN-RFLP and T7E1 assays. Arrows indicate expected positions of DNA bands cleaved by RGEN or T7E1. FIG. 25B: Quantitative comparison of RGEN-RFLP analysis with T7E1 assays. Genomic DNA samples from wild-type and C4BPB-disrupted K562 cells were mixed in various ratios and subjected to PCR amplification. FIG. 25C: Genotyping of RGEN-induced mutations in the HLA-B gene in HeLa cells with RFLP and T7E1 analyses.

FIGS. 26A and 26B show genotyping of mutations induced by engineered nucleases in organisms via RGEN-mediated RFLP. FIG. 26A: Genotype of Pibf1 wild-type (WT) (SEQ ID NO: 242) and the following mutant founder mice: #1 (SEQ ID NO: 243 and SEQ ID NO: 244), #3 (SEQ ID NO: 245 and SEQ ID NO: 246), #4 (SEQ ID NO: 247 and SEQ ID NO: 242), #5 (SEQ ID NO: 246 and SEQ ID NO: 242), #6 (SEQ ID NO: 248 and SEQ ID NO: 249), #8 (SEQ ID NO: 250 and SEQ ID NO: 251), and #11 (SEQ ID NO: 252 and SEQ ID NO: 250). FIG. 26B: Comparison of the mismatch-sensitive T7E1 assay with RGEN-mediated RFLP analysis. Black arrows indicate the cleavage product by treatment of T7E1 enzyme or RGENs.

FIG. 27 shows RGEN-mediated genotyping of ZFN-induced mutations at a wild-type CCR5 sequence (SEQ ID NO: 253). The ZFN target site is shown in box. Black arrows indicate DNA bands cleaved by T7E1.

FIG. 28 shows polymorphic sites in a region of the human HLA-B gene (SEQ ID NO: 254). The sequence, which surrounds the RGEN target site, is that of a PCR amplicon from HeLa cells. Polymorphic positions are shown in box. The RGEN target site and the PAM sequence are shown in dashed and bolded box, respectively. Primer sequences are underlined.

FIGS. 29A and 29B show genotyping of oncogenic mutations via RGEN-RFLP analysis. FIG. 29A: A recurrent mutation (c.133-135 deletion of TCT; SEQ ID NO: 256) in the human CTNNB1 gene in HCT116 cells was detected by RGENs. The wild-type CTNNB1 sequence is represented by SEQ ID NO: 255. HeLa cells were used as a negative control. FIG. 29B: Genotyping of the KRAS substitution mutation (c.34 G>A) in the Δ549 cancer cell line with RGENs that contain mismatched guide RNA that are WT-specific (SEQ ID NO: 257) or mutant-specific (SEQ ID NO: 258). Mismatched nucleotides are shown in box. HeLa cells were used as a negative control. Arrows indicate DNA bands cleaved by RGENs. DNA sequences confirmed by Sanger sequencing are shown: wild-type (SEQ ID NO: 259) and c.34G>A (SEQ ID NO: 260).

FIGS. 30A, 30B, 30C, and 30D show genotyping of the CCR5 delta32 allele in HEK293T cells via RGEN-RFLP analysis. FIG. 30A: RGEN-RFLP assays of cell lines. DNA sequences of the wild-type CCR5 locus (SEQ ID NO: 262) and delta 32 mutation (SEQ ID NO: 261) are shown. K562, SKBR3, and HeLa cells were used as wild-type controls. Arrows indicate DNA bands cleaved by RGENs. FIG. 30B: DNA sequence of wild-type (SEQ ID NO: 263) and delta32 CCR5 alleles (SEQ ID NO: 264). Both on-target and off-target sites of RGENs used in RFLP analysis are underlined. A single-nucleotide mismatch between the two sites is shown in box. The PAM sequence is underlined. FIG. 30C: In vitro cleavage of plasmids harboring WT or del32 CCR5 alleles using the wild-type-specific RGEN. FIG. 30D Confirming the presence of an off-target site of the CCR5-delta32-specific RGEN at the CCR5 locus. In vitro cleavage assays of plasmids harboring either on-target (SEQ ID NO: 265) or off-target sequences (SEQ ID NO: 266) using various amounts of the del32-specific RGEN.

FIGS. 31A and 31B show genotyping of a KRAS point mutation (c.34 G>A). FIG. 31A: RGEN-RFLP analysis of the KRAS mutation (c.34 G>A) in cancer cell lines. PCR products from HeLa cells (used as a wild-type control) or Δ549 cells, which are homozygous for the point mutation, were digested with RGENs with perfectly matched crRNA specific to the wild-type sequence (SEQ ID NO: 259) or the mutant sequence (SEQ ID NO: 260). KRAS genotypes in these cells were confirmed by Sanger sequencing. FIG. 31B: Plasmids harboring either the wild-type (SEQ ID NO: 259) or mutant KRAS sequences (SEQ ID NO: 260) were digested using RGENs with perfectly matched crRNAs or attenuated, one-base mismatched crRNAs: m7 (SEQ ID NO: 267), m6 (SEQ ID NO: 257), m5 (SEQ ID NO: 268), m4 (SEQ ID NO: 269), m8 (SEQ ID NO: 260), m7,8 (SEQ ID NO: 270), m6,8 (SEQ ID NO: 258), m5,8 (SEQ ID NO: 271), and m4,8 (SEQ ID NO: 272). Attenuated crRNAs that were chosen for genotyping are labeled in box above the gels.

FIGS. 32A and 32B show genotyping of a PIK3CA point mutation (c.3140 A>G). FIG. 32A: RGEN-RFLP analysis of the PIK3CA mutation (c.3140 A>G) in cancer cell lines. PCR products from HeLa cells (used as a wild-type control) or HCT116 cells that are heterozygous for the point mutation were digested with RGENs with perfectly matched crRNA specific to the wild-type sequence (SEQ ID NO: 273) or the mutant sequence (SEQ ID NO: 274). PIK3CA genotypes in these cells were confirmed by Sanger sequencing. FIG. 32B: Plasmids harboring either the wild-type PIK3CA sequence (SEQ ID NO: 273) or mutant PIK3CA sequence (SEQ ID NO: 274) were digested using RGENs with perfectly matched crRNAs or attenuated, one-base mismatched crRNAs: m5 (SEQ ID NO: 275), m6 (SEQ ID NO: 276), m7 (SEQ ID NO: 277), m10 (SEQ ID NO: 278), m13 (SEQ ID NO: 279), m16 (SEQ ID NO: 280), m19 (SEQ ID NO: 281), m4 (SEQ ID NO:274), m4,5 (SEQ ID NO: 282), m4,6 (SEQ ID NO: 283), m4,7 (SEQ ID NO: 284), m4,10 (SEQ ID NO: 285), m4,13 (SEQ ID NO: 286), m4,16 (SEQ ID NO: 287), and m4,19 (SEQ ID NO: 288). Attenuated crRNAs that were chosen for genotyping are labeled in box above the gels.

FIGS. 33A, 33B, 33C, and 33D show genotyping of recurrent point mutations in cancer cell lines. FIG. 33A: RGEN-RFLP assays to distinguish between a wild-type IDH gene sequence (SEQ ID NO: 289) and a recurrent oncogenic point mutation sequence in the IDH gene (c.394c>T; SEQ ID NO: 290). RGENs with attenuated, one-base mismatched crRNAs, SEQ ID NO: 291 (WT-Specific RNA) and SEQ ID NO: 292 (Mutant-Specific RNA), distinguished the wild type and mutant IDH sequences. FIG. 33B: RGEN-RFLP assays to distinguish between a wild-type PIK3CA gene sequence (SEQ ID NO: 271) and a recurrent oncogenic point mutation sequence in the PIK3CA gene (c.3140A>G; SEQ ID NO: 273). RGENs with attenuated, one-base mismatched crRNAs, SEQ ID NO: 275 (WT-Specific RNA) and SEQ ID NO: 284 (Mutant-Specific RNA), distinguished the wild type and mutant PIK3CA sequences. FIG. 33C: RGEN-RFLP assays to distinguish between a wild-type NRAS gene sequence (SEQ ID NO: 293) and a recurrent oncogenic point mutation sequence in the NRAS gene (c.181C>A; SEQ ID NO: 294). RGENs with perfectly matched crRNAs, SEQ ID NO: 293 (WT-Specific RNA) and SEQ ID NO: 294 (Mutant-Specific RNA), distinguished the wild type and mutant NRAS sequences. FIG. 33D: RGEN-RFLP assays to distinguish between a wild-type BRAF gene sequence (SEQ ID NO: 295) and a recurrent oncogenic point mutation sequence in the BRAF gene (c.1799T>A; SEQ ID NO: 296). RGENs with perfectly matched crRNAs, SEQ ID NO: 295 (WT-Specific RNA) and SEQ ID NO: 296 (Mutant-Specific RNA), distinguished the wild type and mutant BRAF sequences. Genotypes of each cell line confirmed by Sanger sequencing are shown. Mismatched nucleotides are shown in box. Black arrows indicate DNA bands cleaved by RGENs.

### BEST MODE FOR CARRYING OUT THE INVENTION

In accordance with one aspect of the invention, the present invention provides a composition for cleaving target DNA in eukaryotic cells or organisms comprising a guide RNA specific for target DNA or DNA that encodes the guide RNA, and Cas protein-encoding nucleic acid or Cas protein. In addition, the present invention provides a use of the composition for cleaving target DNA in eukaryotic cells or organisms comprising a guide RNA specific for target DNA or DNA that encodes the guide RNA, and Cas protein-encoding nucleic acid or Cas protein.

In the present invention, the composition is also referred to as a RNA-guided endonuclease (RGEN) composition.

ZFNs and TALENs enable targeted mutagenesis in mammalian cells, model organisms, plants, and livestock, but the mutation frequencies obtained with individual nucleases are widely different from each other. Furthermore, some ZFNs and TALENs fail to show any genome editing activities. DNA methylation may limit the binding of these engineered nucleases to target sites. In addition, it is technically challenging and time-consuming to make customized nucleases.

The present inventors have developed a new RNA-guided endonuclease composition based on Cas protein to overcome the disadvantages of ZFNs and TALENs.

Prior to the present invention, an endonuclease activity of Cas proteins has been known. However, it has not been known whether the endonuclease activity of Cas protein would function in an eukaryotic cell because of the complexity of the eukaryotic genome. Further, until now, a composition comprising Cas protein or Cas protein-encoding nucleic acid and a guide RNA specific for the target DNA to cleave a target DNA in eukaryotic cells or organisms has not been developed.

Compared to ZFNs and TALENs, the present RGEN composition based on Cas protein can be more readily customized because only the synthetic guide RNA component is replaced to make a new genome-editing nuclease. No sub-cloning steps are involved to make customized RNA guided endonucleases. Furthermore, the relatively small size of the Cas gene (for example, 4.2 kbp for Cas9) as compared to a pair of TALEN genes (~6 kbp) provides an advantage for this RNA-guided endonuclease composition in some applications such as virus-mediated gene delivery. Further, this RNA-guided endonuclease does not have off-target effects and thus does not induce unwanted mutations, deletion, inversions, and duplications. These features make the present RNA-guided endonuclease composition a scalable, versatile, and convenient tool for genome engineering in eukaryotic cells and organisms. In addition, RGEN can be designed to target any DNA sequence, almost any single nucleotide polymorphism or small insertion/deletion (indel) can be analyzed via RGEN-mediated RFLP. The specificity of RGENs is determined by the RNA component that hybridizes with a target DNA sequence of up to 20 base pairs (bp) in length and by the Cas9 protein that recognizes the protospacer-adjacent motif (PAM). RGENs are readily reprogrammed by replacing the RNA component. Therefore, RGENs provide a platform to use simple and robust R00FLP analysis for various sequence variations.

The target DNA may be an endogenous DNA, or artificial DNA, preferably, endogenous DNA.

As used herein, the term “Cas protein” refers to an essential protein component in the CRISPR/Cas system, forms an active endonuclease or nickase when complexed with two RNAs termed CRISPR RNA (crRNA) and trans-activating crRNA (tracrRNA).

The information on the gene and protein of Cas are available from GenBank of National Center for Biotechnology Information (NCBI), without limitation.

The CRISPR-associated (cas) genes encoding Cas proteins are often associated with CRISPR repeat-spacer arrays. More than forty different Cas protein families have been described. Of these protein families, Cas1 appears to be ubiquitous among different CRISPR/Cas systems. There are three types of CRISPR-Cas system. Among them, Type II CRISPR/Cas system involving Cas9 protein and crRNA and tracrRNA is representative and is well known. Particular combinations of cas genes and repeat structures have been used to define 8 CRISPR subtypes (Ecoli, Ypest, Nmeni, Dvulg, Tneap, Hmari, Apern, and Mtube).

The Cas protein may be linked to a protein transduction domain. The protein transduction domain may be poly-arginine or a TAT protein derived from HIV, but it is not limited thereto.

The present composition may comprise Cas component in the form of a protein or in the form of a nucleic acid encoding Cas protein.

In the present invention, Cas protein may be any Cas protein provided that it has an endonuclease or nickase activity when complexed with a guide RNA.

Preferably, Cas protein is Cas9 protein or variants thereof.

The variant of the Cas9 protein may be a mutant form of Cas9 in which the catalytic aspartate residue is changed to any other amino acid. Preferably, the other amino acid may be an alanine, but it is not limited thereto.

Further, Cas protein may be the one isolated from an organism such as Streptococcus sp., preferably Streptococcus pyogenes or a recombinant protein, but it is not limited thereto.

The Cas protein derived from Streptococcus pyogenes may recognize NGG trinucleotide. The Cas protein may comprise an amino acid sequence of SEQ ID NO: 109, but it is not limited thereto.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, a recombinant Cas protein may be generated by reconstituting Cas protein-encoding sequence using the human codon table.

As for the present invention, Cas protein-encoding nucleic acid may be a form of vector, such as plasmid comprising Cas-encoding sequence under a promoter such as CMV or CAG. When Cas protein is Cas9, Cas9 encoding sequence may be derived from Streptococcus sp., and preferably derived from Streptococcus pyogenes. For example, Cas9 encoding nucleic acid may comprise the nucleotide sequence of SEQ ID. NO: 1. Moreover, Cas9 encoding nucleic acid may comprise the nucleotide sequence having homology of at least 50% to the sequence of SEQ ID NO: 1, preferably at least 60, 70, 80, 90, 95, 97, 98, or 99% to the SEQ ID NO:1, but it is not limited thereto. Cas9 encoding nucleic acid may comprise the nucleotide sequence of SEQ ID NOs.108, 110, 106, or 107.

As used herein, the term “guide RNA” refers to a RNA which is specific for the target DNA and can form a complex with Cas protein and bring Cas protein to the target DNA.

In the present invention, the guide RNA may consist of two RNA, i.e., CRISPR RNA (crRNA) and transactivating crRNA (tracrRNA) or be a single-chain RNA (sgRNA) produced by fusion of an essential portion of crRNA and tracrRNA.

The guide RNA may be a dualRNA comprising a crRNA and a tracrRNA.

If the guide RNA comprises the essential portion of crRNA and tracrRNA and a portion complementary to a target, any guide RNA may be used in the present invention.

The crRNA may hybridize with a target DNA.

The RGEN may consist of Cas protein, and dualRNA (invariable tracrRNA and target-specific crRNA), or Cas protein and sgRNA (fusion of an essential portion of invariable tracrRNA and target-specific crRNA), and may be readily reprogrammed by replacing crRNA.

The guide RNA further comprises one or more additional nucleotides at the 5′ end of the single-chain guide RNA or the crRNA of the dualRNA.

Preferably, the guide RNA further comprises 2-additional guanine nucleotides at the 5′ end of the single-chain guide RNA or the crRNA of the dualRNA.

The guide RNA may be transferred into a cell or an organism in the form of RNA or DNA that encodes the guide RNA.

The guide RNA may be in the form of an isolated RNA, RNA incorporated into a viral vector, or is encoded in a vector. Preferably, the vector may be a viral vector, plasmid vector, or agrobacterium vector, but it is not limited thereto.

A DNA that encodes the guide RNA may be a vector comprising a sequence coding for the guide RNA. For example, the guide RNA may be transferred into a cell or organism by transfecting the cell or organism with the isolated guide RNA or plasmid DNA comprising a sequence coding for the guide RNA and a promoter.

Alternatively, the guide RNA may be transferred into a cell or organism using virus-mediated gene delivery.

When the guide RNA is transfected in the form of an isolated RNA into a cell or organism, the guide RNA may be prepared by in vitro transcription using any in vitro transcription system known in the art. The guide RNA is preferably transferred to a cell in the form of isolated RNA rather than in the form of plasmid comprising encoding sequence for a guide RNA. As used herein, the term “isolated RNA” may be interchangeable to “naked RNA”. This is cost- and time-saving because it does not require a step of cloning. However, the use of plasmid DNA or virus-mediated gene delivery for transfection of the guide RNA is not excluded.

The present RGEN composition comprising Cas protein or Cas protein-encoding nucleic acid and a guide RNA can specifically cleave a target DNA due to a specificity of the guide RNA for a target and an endonuclease or nickase activity of Cas protein.

As used herein, the term “cleavage” refers to the breakage of the covalent backbone of a nucleotide molecule.

In the present invention, a guide RNA may be prepared to be specific for any target which is to be cleaved. Therefore, the present RGEN composition can cleave any target DNA by manipulating or genotyping the target-specific portion of the guide RNA.

The guide RNA and the Cas protein may function as a pair. As used herein, the term “paired Cas nickase” may refer to the guide RNA and the Cas protein functioning as a pair. The pair comprises two guide RNAs. The guide RNA and Cas protein may function as a pair, and induce two nicks on different DNA strand. The two nicks may be separated by at least 100 bps, but are not limited thereto.

In the Example, the present inventors confirmed that paired Cas nickase allow targeted mutagenesis and large deletions of up to 1-kbp chromosomal segments in human cells. Importantly, paired nickases did not induce indels at off-target sites at which their corresponding nucleases induce mutations. Furthermore, unlike nucleases, paired nickases did not promote unwanted translocations associated with off-target DNA cleavages. In principle, paired nickases double the specificity of Cas9-mediated mutagenesis and will broaden the utility of RNA-guided enzymes in applications that require precise genome editing such as gene and cell therapy.

In the present invention, the composition may be used in the genotyping of a genome in the eukaryotic cells or organisms in vitro.

In one specific embodiment, the guide RNA may comprise the nucleotide sequence of Seq ID. No. 1, wherein the portion of nucleotide position 3~22 is a target-specific portion and thus, the sequence of this portion may be changed depending on a target.

As used herein, a eukaryotic cell or organism may be yeast, fungus, protozoa, plant, higher plant, and insect, or amphibian cells, or mammalian cells such as CHO, HeLa, HEK293, and COS-1, for example, cultured cells (in vitro), graft cells and primary cell culture (in vitro and ex vivo), and in vivo cells, and also mammalian cells including human, which are commonly used in the art, without limitation.

In one specific embodiment, it was found that Cas9 protein/single-chain guide RNA could generate site-specific DNA double-strand breaks in vitro and in mammalian cells, whose spontaneous repair induced targeted genome mutations at high frequencies.

Moreover, it was found that gene-knockout mice could be induced by the injection of Cas9 protein/guide RNA complexes or Cas9 mRNA/guide RNA into one-cell stage embryo and germ-line transmittable mutations could be generated by Cas9/guide RNA system.

Using Cas protein rather than a nucleic acid encoding Cas protein to induce a targeted mutagenesis is advantageous because exogeneous DNA is not introduced into an organism. Thus, the composition comprising Cas protein and a guide RNA may be used to develop therapeutics or value-added crops, livestock, poultry, fish, pets, etc.

In accordance with another aspect of the invention, the present invention provides a composition for inducing targeted mutagenesis in eukaryotic cells or organisms, comprising a guide RNA specific for target DNA or DNA that encodes the guide RNA, and Cas protein-encoding nucleic acid or Cas protein. In addition, the present invention provides a use of the composition for inducing targeted mutagenesis in eukaryotic cells or organisms, comprising a guide RNA specific for target DNA or DNA that encodes the guide RNA, and Cas protein-encoding nucleic acid or Cas protein.

A guide RNA, Cas protein-encoding nucleic acid or Cas protein are as described in the above.

In accordance with another aspect of the invention, the present invention provides a kit for cleaving a target DNA or inducing targeted mutagenesis in eukaryotic cells or organisms comprising a guide RNA specific for target DNA or DNA that encodes the guide RNA, and Cas protein-encoding nucleic acid or Cas protein.

A guide RNA, Cas protein-encoding nucleic acid or Cas protein are as described in the above.

The kit may comprise a guide RNA and Cas protein-encoding nucleic acid or Cas protein as separate components or as one composition.

The present kit may comprise some additional components necessary for transferring the guide RNA and Cas component to a cell or an organism. For example, the kit may comprise an injection buffer such as DEPC-treated injection buffer, and materials necessary for analysis of mutation of a target DNA, but are not limited thereto.

In accordance with another aspect, the present invention provides a method for preparing a eukaryotic cell or organism comprising Cas protein and a guide RNA comprising a step of co-transfecting or serial-transfecting the eukaryotic cell or organism with a Cas protein-encoding nucleic acid or Cas protein, and a guide RNA or DNA that encodes the guide RNA.

A guide RNA, Cas protein-encoding nucleic acid or Cas protein are as described in the above.

In the present invention, a Cas protein-encoding nucleic acid or Cas protein and a guide RNA or DNA that encodes the guide RNA may be transferred into a cell by various methods known in the art, such as microinjection, electroporation, DEAE-dextran treatment, lipofection, nanoparticle-mediated transfection, protein transduction domain mediated transduction, virus-mediated gene delivery, and PEG-mediated transfection in protoplast, and so on, but are not limited thereto. Also, a Cas protein encoding nucleic acid or Cas protein and a guide RNA may be transferred into an organism by various method known in the art to administer a gene or a protein such as injection. A Cas protein-encoding nucleic acid or Cas protein may be transferred into a cell in the form of complex with a guide RNA, or separately. Cas protein fused to a protein transduction domain such as Tat can also be delivered efficiently into cells.

Preferably, the eukaryotic cell or organism is co-transfected or serial-transfected with a Cas9 protein and a guide RNA.

The serial-transfection may be performed by transfection with Cas protein-encoding nucleic acid first, followed by second transfection with naked guide RNA. Preferably, the second transfection is after 3, 6, 12, 18, 24 hours, but it is not limited thereto.

In accordance with another aspect, the present invention provides a eukaryotic cell or organism comprising a guide RNA specific for target DNA or DNA that encodes the guide RNA, and Cas protein-encoding nucleic acid or Cas protein.

The eukaryotic cells or organisms may be prepared by transferring the composition comprising a guide RNA specific for target DNA or DNA that encodes the guide RNA, and Cas protein-encoding nucleic acid or Cas protein into the cell or organism.

The eukaryotic cell may be yeast, fungus, protozoa, higher plant, and insect, or amphibian cells, or mammalian cells such as CHO, HeLa, HEK293, and COS-1, for example, cultured cells (in vitro), graft cells and primary cell culture (in vitro and ex vivo), and in vivo cells, and also mammalian cells including human, which are commonly used in the art, without limitation. Further the organism may be yeast, fungus, protozoa, plant, higher plant, insect, amphibian, or mammal.

In accordance with another aspect of the invention, the present invention provides a method for cleaving a target DNA or inducing targeted mutagenesis in eukaryotic cells or organisms, comprising a step of treating a cell or organism comprising a target DNA with a composition comprising a guide RNA specific for target DNA or DNA that encodes the guide RNA, and Cas protein-encoding nucleic acid or Cas protein.

The step of treating a cell or organism with the composition may be performed by transferring the present composition comprising a guide RNA specific for target DNA or DNA that encodes the guide RNA, and Cas protein-encoding nucleic acid or Cas protein into the cell or organism.

As described in the above, such transfer may be performed by microinjection, transfection, electroporation, and so on.

In accordance with another aspect of the invention, the present invention provides an embryo comprising a genome edited by the present RGEN composition comprising a guide RNA specific for target DNA or DNA that encodes the guide RNA, and Cas protein-encoding nucleic acid or Cas protein.

Any embryo can be used in the present invention, and for the present invention, the embryo may be an embryo of a mouse. The embryo may be produced by injecting PMSG (Pregnant Mare Serum Gonadotropin) and hCG (human Chorionic Gonadotropin) into a female mouse of 4 to 7 weeks and the super-ovulated female mouse may be mated to males, and the fertilized embryos may be collected from oviducts.

The present RGEN composition introduced into an embryo can cleave a target DNA complementary to the guide RNA by the action of Cas protein and cause a mutation in the target DNA. Thus, the embryo into which the present RGEN composition has been introduced has an edited genome.

In one specific embodiment, it was found that the present RGEN composition could cause a mutation in a mouse embryo and the mutation could be transmitted to offspring.

A method for introducing the RGEN composition into the embryo may be any method known in the art, such as microinjection, stem cell insertion, retrovirus insertion, and so on. Preferably, a microinjection technique can be used.

In accordance with another aspect, the present invention provides a genome-modified animal obtained by transferring the embryo comprising a genome edited by the present RGEN composition into the oviducts of an animal.

In the present invention, the term “genome-modified animal” refers to an animal of which genome has been modified in the stage of embryo by the present RGEN composition and the type of the animal is not limited.

The genome-modified animal has mutations caused by a targeted mutagenesis based on the present RGEN composition.

The mutations may be any one of deletion, insertion, translocation, inversion. The site of mutation depends on the sequence of guide RNA of the RGEN composition.

The genome-modified animal having a mutation of a gene may be used to determine the function of the gene.

In accordance with another aspect of the invention, the present invention provides a method of preparing a genome-modified animal comprising a step of introducing the present RGEN composition comprising a guide RNA specific for the target DNA or DNA that encodes the guide RNA and Cas protein-encoding nucleic acid or Cas protein into an embryo of an animal; and a step of transferring the embryo into a oviduct of pseudopregnant foster mother to produce a genome-modified animal.

The step of introducing the present RGEN composition may be accomplished by any method known in the art such as microinjection, stem cell insertion, retroviral insertion, and so on.

In accordance with another aspect of the invention, the present invention provides a plant regenerated form the genome-modified protoplasts prepared by the method for eukaryotic cells comprising the RGEN composition.

In accordance with another aspect of the invention, the present invention provides a composition for genotyping mutations or variations in an isolated biological sample, comprising a guide RNA specific for the target DNA sequence Cas protein. In addition, the present invention provides a composition for genotyping nucleic acid sequences in pathogenic microorganisms in an isolated biological sample, comprising a guide RNA specific for the target DNA sequence and Cas protein.

A guide RNA, Cas protein-encoding nucleic acid or Cas protein are as described in the above.

As used herein the term “genotyping” refers to the “Restriction fragment length polymorphism (RFLP) assay”.

RFLP may be used in 1) the detection of indel in cells or organisms induced by the engineered nucleases, 2) the genotyping naturally-occurring mutations or variations in cells or organisms, or 3) the genotyping the DNA of infected pathogenic microorganisms including virus or bacteria, etc.

The mutations or variation may be induced by engineered nucleases in cells.

The engineered nuclease may be a Zinc Finger Nuclease (ZFNs), Transcription Activator-Like Effector Nucleases (TALENs), or RGENs, but it is not limited thereto.

As used herein the term “biological sample” includes samples for analysis, such as tissues, cells, whole blood, serum, plasma, saliva, sputum, cerebrospinal fluid or urine, but is not limited thereto.

The mutations or variation may be a naturally-occurring mutations or variations.

The mutations or variations are induced by the pathogenic microorganisms. Namely, the mutations or variations occur due to the infection of pathogenic microorganisms, when the pathogenic microorganisms are detected, the biological sample is identified as infected.

The pathogenic microorganisms may be virus or bacteria, but are not limited thereto.

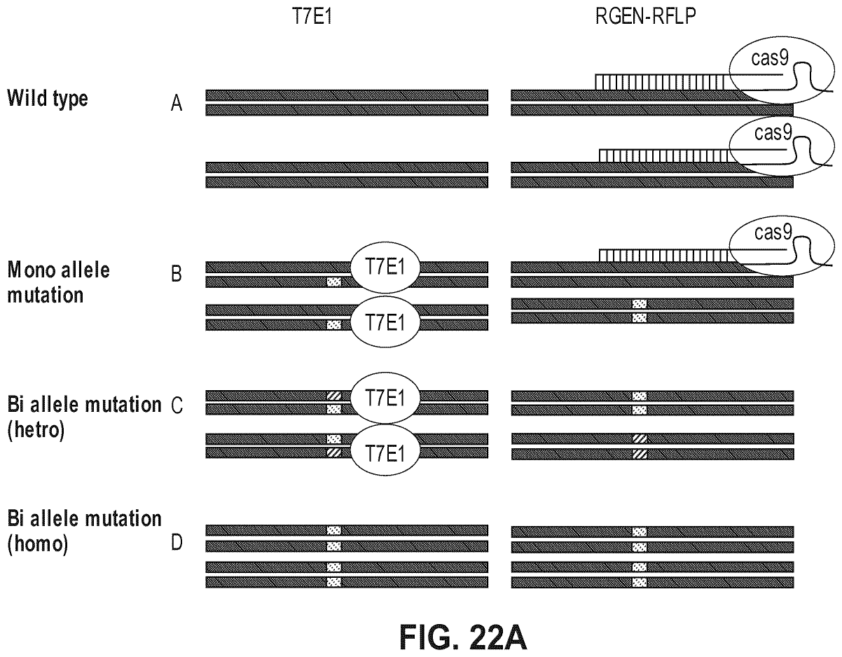

Engineered nuclease-induced mutations are detected by various methods, which include mismatch-sensitive Surveyor or T7 endonuclease I (T7E1) assays, RFLP analysis, fluorescent PCR, DNA melting analysis, and Sanger and deep sequencing. The T7E1 and Surveyor assays are widely used but often underestimate mutation frequencies because the assays detect heteroduplexes (formed by the hybridization of mutant and wild-type sequences or two different mutant sequences); they fail to detect homoduplexes formed by the hybridization of two identical mutant sequences. Thus, these assays cannot distinguish homozygous biallelic mutant clones from wild-type cells nor heterozygous biallelic mutants from heterozygous monoallelic mutants (FIG. 22). In addition, sequence polymorphisms near the nuclease target site can produce confounding results because the enzymes can cleave heteroduplexes formed by hybridization of these different wild-type alleles. RFLP analysis is free of these limitations and therefore is a method of choice. Indeed, RFLP analysis was one of the first methods used to detect engineered nuclease-mediated mutations. Unfortunately, however, it is limited by the availability of appropriate restriction sites.

In accordance with another aspect of the invention, the present invention provides a kit for genotyping mutations or variations in an isolated biological sample, comprising the composition for genotyping mutations or variations in an isolated biological sample. In addition, the present invention provides a kit for genotyping nucleic acid sequences in pathogenic microorganisms in an isolated biological sample, comprising a guide RNA specific for the target DNA sequence and Cas protein.

A guide RNA, Cas protein-encoding nucleic acid or Cas protein are as described in the above.

In accordance with another aspect of the invention, the present invention provides a method of genotyping mutations or variations in an isolated biological sample, using the composition for genotyping mutations or variations in an isolated biological sample. In addition, the present invention provides a method of genotyping nucleic acid sequences in pathogenic microorganisms in an isolated biological sample, comprising a guide RNA specific for the target DNA sequence and Cas protein.

A guide RNA, Cas protein-encoding nucleic acid or Cas protein are as described in the above.

### MODE FOR THE INVENTION

Hereinafter, the present invention will be described in more detail with reference to Examples. However, these Examples are for illustrative purposes only, and the invention is not intended to be limited by these Examples.

### Example 1: Genome Editing Assay

### 1-1. DNA Cleavage Activity of Cas9 Protein

Firstly, the DNA cleavage activity of Cas9 derived from Streptococcus pyogenes in the presence or absence of a chimeric guide RNA in vitro was tested.

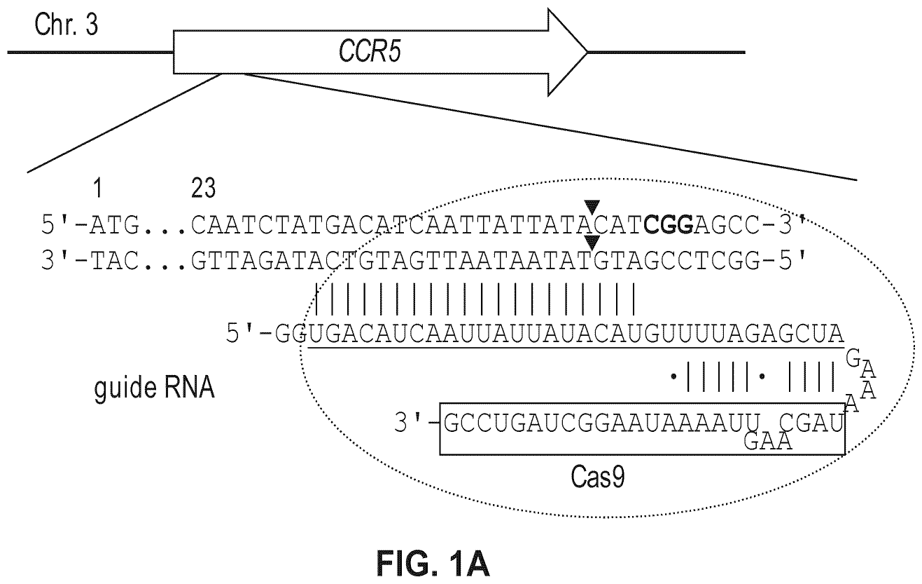

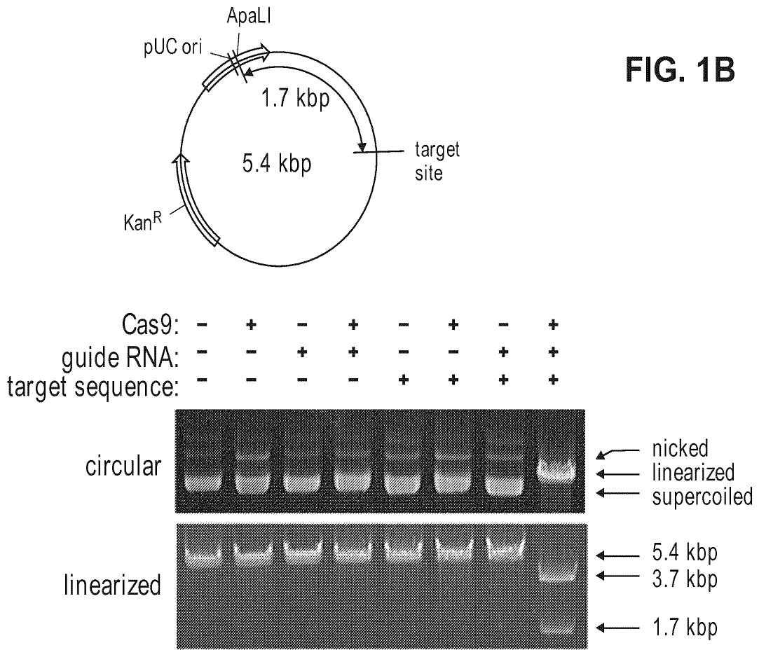

To this end, recombinant Cas9 protein that was expressed in and purified from E. coli was used to cleave a predigested or circular plasmid DNA that contained the 23-base pair (bp) human CCR5 target sequence. A Cas9 target sequence consists of a 20-bp DNA sequence complementary to crRNA or a chimeric guide RNA and the trinucleotide (5′-NGG-3′) protospacer adjacent motif (PAM) recognized by Cas9 itself (FIG. 1A).

Specifically, the Cas9-coding sequence (4,104 bp), derived from Streptococcus pyogenes strain M1 GAS (NC_002737.1), was reconstituted using the human codon usage table and synthesized using oligonucleotides. First, 1-kb DNA segments were assembled using overlapping ~35-mer oligonucleotides and Phusion™ polymerase (New England Biolabs) and cloned into T-vector (SolGent). A full-length Cas9 sequence was assembled using four 1-kbp DNA segments by overlap PCR. The Cas9-encoding DNA segment was subcloned into p3s, which was derived from pcDNA3.1 (Invitrogen). In this vector, a peptide tag (NH2-GGSGPPKKKRKVYPYDVPDYA-COOH, SEQ ID NO: 2) containing the HA epitope and a nuclear localization signal (NLS) was added to the C-terminus of Cas9. Expression and nuclear localization of the Cas9 protein in HEK 293T cells were confirmed by western blotting using anti-HA antibody (Santa Cruz).

Then, the Cas9 cassette was subcloned into pET28-b(+) and transformed into BL21(DE3). The expression of Cas9 was induced using 0.5 mM IPTG for 4 h at 25° C. The Cas9 protein containing the His6-tag at the C terminus was purified using Ni-NTA agarose resin (Qiagen) and dialyzed against 20 mM HEPES (pH 7.5), 150 mM KCl, 1 mM DTT, and 10% glycerol (1). Purified Cas9 (50 nM) was incubated with super-coiled or pre-digested plasmid DNA (300 ng) and chimeric RNA (50 nM) in a reaction volume of 20 μl in NEB buffer 3 for 1 h at 37° C. Digested DNA was analyzed by electrophoresis using 0.8% agarose gels.

Cas9 cleaved the plasmid DNA efficiently at the expected position only in the presence of the synthetic RNA and did not cleave a control plasmid that lacked the target sequence (FIG. 1B).

### 1-2. DNA Cleavage by Cas9/Guide RNA Complex in Human Cells

A RFP-GFP reporter was used to investigate whether the Cas9/guide RNA complex can cleave the target sequence incorporated between the RFP and GFP sequences in mammalian cells.

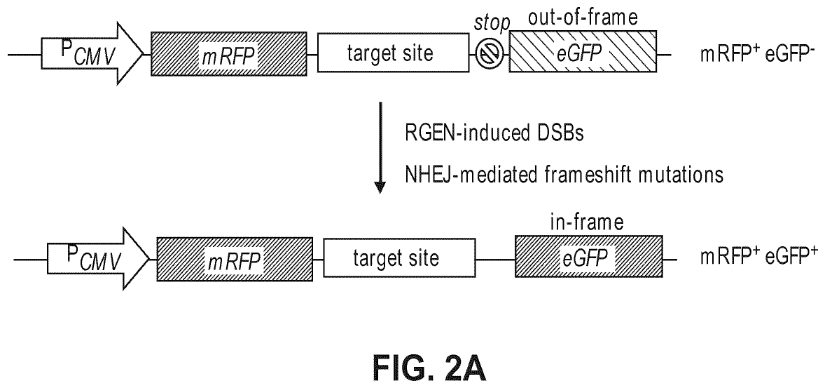

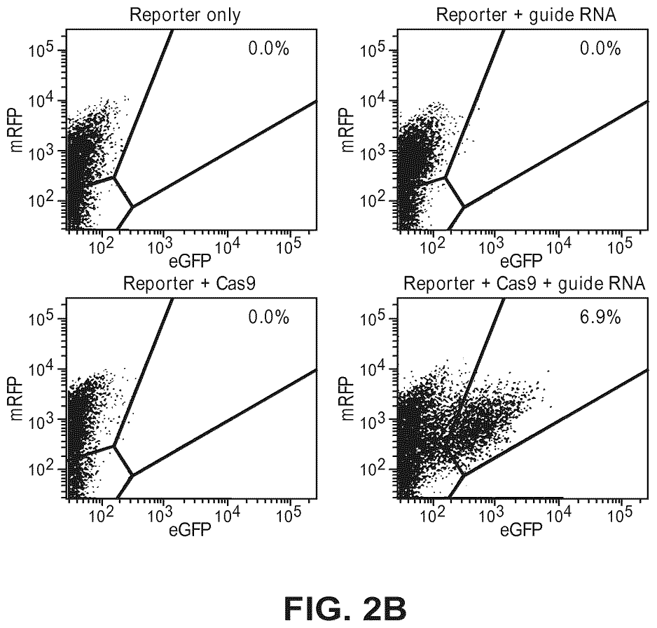

In this reporter, the GFP sequence is fused to the RFP sequence out-of-frame (2). The active GFP is expressed only when the target sequence is cleaved by site-specific nucleases, which causes frameshifting small insertions or deletions (indels) around the target sequence via error-prone non-homologous end-joining (NHEJ) repair of the double-strand break (DSB) (FIG. 2).

The RFP-GFP reporter plasmids used in this study were constructed as described previously (2). Oligonucleotides corresponding to target sites (Table 1) were synthesized (Macrogen) and annealed. The annealed oligonucleotides were ligated into a reporter vector digested with EcoRI and BamHI.

HEK 293T cells were co-transfected with Cas9-encoding plasmid (0.8 μg) and the RFP-GFP reporter plasmid (0.2 μg) in a 24-well plate using Lipofectamine™ 2000 (Invitrogen).

Meanwhile, the in vitro transcribed chimeric RNA had been prepared as follows. RNA was in vitro transcribed through run-off reactions using the MEGAshortscript™ T7 kit (Ambion) according to the manufacturer's manual. Templates for RNA in vitro transcription were generated by annealing two complementary single strand DNAs or by PCR amplification (Table 1). Transcribed RNA was resolved on a 8% denaturing urea-PAGE gel. The gel slice containing RNA was cut out and transferred to probe elution buffer. RNA was recovered in nuclease-free water followed by phenol:chloroform extraction, chloroform extraction, and ethanol precipitation. Purified RNAs were quantified by spectrometry.

At 12 h post transfection, chimeric RNA (1 μg) prepared by in vitro transcription was transfected using Lipofectamine 2000.

At 3d post-transfection, transfected cells were subjected to flow cytometry and cells expressing both RFP and GFP were counted.

It was found that GFP-expressing cells were obtained only when the cells were transfected first with the Cas9 plasmid and then with the guide RNA 12 h later (FIG. 2), demonstrating that RGENs could recognize and cleave the target DNA sequence in cultured human cells. Thus GFP-expressing cells were obtained by serial-transfection of the Cas9 plasmid and the guide RNA rather than co-transfection.

TABLE 1

| | | |
|---|---|---|
| Gene | sequence (5′ to 3′) | SEQ ID NO. |

Oligonucleotides used for the construction of the reporter plasmid

| | | | |
|---|---|---|---|
| CCR5 | F | AATTCATGACATCAATTATTATACATCGGAGGAG | 3 |
| | R | GATCCTCCTCCGATGTATAATAATTGATGTCATG | 4 |

Primers used in the T7E1 assay

| | | | |
|---|---|---|---|
| CCR5 | F1 | CTCCATGGTGCTATAGAGCA | 5 |
| | F2 | GAGCCAAGCTCTCCATCTAGT | 6 |
| | R | GCCCTGTCAAGAGTTGACAC | 7 |
| C4BPB | F1 | TATTTGGCTGGTTGAAAGGG | 8 |
| | R1 | AAAGTCATGAAATAAACACACCCA | 9 |
| | F2 | CTGCATTGATATGGTAGTACCATG | 10 |
| | R2 | GCTGTTCATTGCAATGGAATG | 11 |

Primers used for the amplification of off-target sites

| | | | |
|---|---|---|---|
| ADCY5 | F1 | GCTCCCACCTTAGTGCTCTG | 12 |
| | R1 | GGTGGCAGGAACCTGTATGT | 13 |
| | F2 | GTCATTGGCCAGAGATGTGGA | 14 |
| | R2 | GTCCCATGACAGGCGTGTAT | 15 |
| KCNJ6 | F | GCCTGGCCAAGTTTCAGTTA | 16 |
| | R1 | TGGAGCCATTGGTTTGCATC | 17 |
| | R2 | CCAGAACTAAGCCGTTTCTGAC | 18 |
| CNTNAP2 | F1 | ATCACCGACAACCAGTTTCC | 19 |
| | F2 | TGCAGTGCAGACTCTTTCCA | 20 |
| | R | AAGGACACAGGGCAACTGAA | 21 |
| N/A Chr.5 | F1 | TGTGGAACGAGTGGTGACAG | 22 |
| | R1 | GCTGGATTAGGAGGCAGGATTC | 23 |
| | F2 | GTGCTGAGAACGCTTCATAGAG | 24 |
| | R2 | GGACCAAACCACATTCTTCTCAC | 25 |

Primers used for the detection of chromosomal deletions

| | | | |
|---|---|---|---|
| Deletion | F | CCACATCTCGTTCTCGGTTT | 26 |
| | R | TCACAAGCCCACAGATATTT | 27 |

### 1-3. Targeted Disruption of Endogenous Genes in Mammalian Cells by RGEN

To test whether RGENs could be used for targeted disruption of endogenous genes in mammalian cells, genomic DNA isolated from transfected cells using T7 endonuclease I (T7E1), a mismatch-sensitive endonuclease that specifically recognizes and cleaves heteroduplexes formed by the hybridization of wild-type and mutant DNA sequences was analyzed (3).

To introduce DSBs in mammalian cells using RGENs, 2×10⁶ K562 cells were transfected with 20 μg of Cas9-encoding plasmid using the 4D-Nucleofector™, SF Cell Line 4D-Nucleofector® X Kit, Program FF-120 (Lonza) according to the manufacturer's protocol. For this experiment, K562 (ATCC, CCL-243) cells were grown in RPMI-1640 with 10% FBS and the penicillin/streptomycin mix (100 U/ml and 100 μg/ml, respectively).

After 24 h, 10-40 μg of in vitro transcribed chimeric RNA was nucleofected into 1×10⁶ K562 cells. The in vitro transcribed chimeric RNA had been prepared as described in the Example 1-2.

Cells were collected two days after RNA transfection and genomic DNA was isolated. The region including the target site was PCR-amplified using the primers described in Table 1. The amplicons were subjected to the T7E1 assay as described previously (3). For sequencing analysis, PCR products corresponding to genomic modifications were purified and cloned into the T-Blunt vector using the T-Blunt PCR Cloning Kit (SolGent). Cloned products were sequenced using the M13 primer.

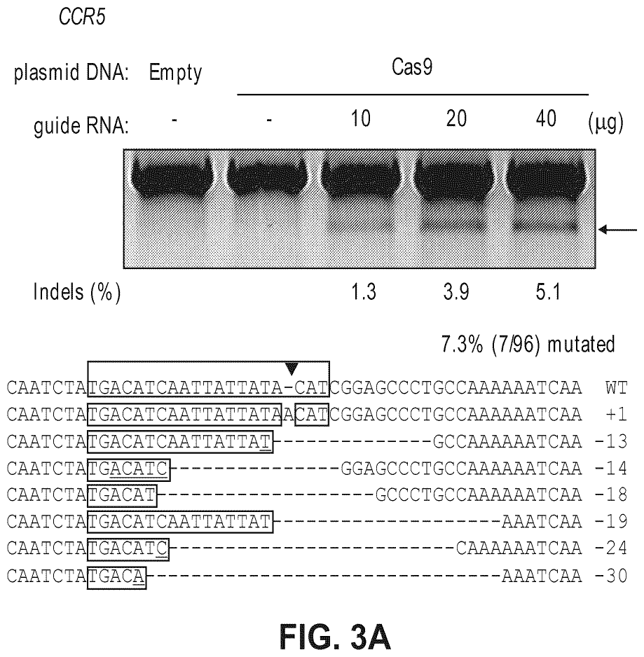

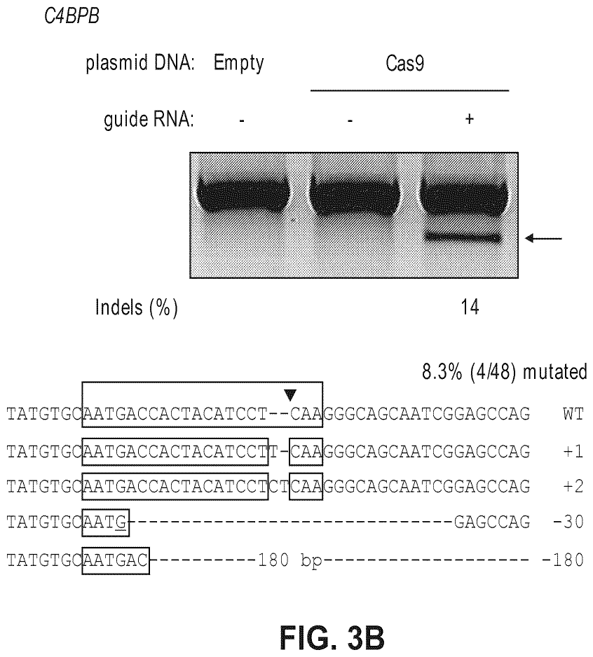

It was found that mutations were induced only when the cells were transfected serially with Cas9-encoding plasmid and then with guide RNA (FIG. 3). Mutation frequencies (Indels (%) in FIG. 3A) estimated from the relative DNA band intensities were RNA-dosage dependent, ranging from 1.3% to 5.1%. DNA sequencing analysis of the PCR amplicons corroborated the induction of RGEN-mediated mutations at the endogenous sites.

Indels and microhomologies, characteristic of error-prone NHEJ, were observed at the target site. The mutation frequency measured by direct sequencing was 7.3% (=7 mutant clones/96 clones), on par with those obtained with zinc finger nucleases (ZFNs) or transcription-activator-like effector nucleases (TALENs).

Serial-transfection of Cas9 plasmid and guide RNA was required to induce mutations in cells. But when plasmids that encode guide RNA, serial transfection was unnecessary and cells were co-transfected with Cas9 plasmid and guide RNA-encoding plasmid.

In the meantime, both ZFNs and TALENs have been successfully developed to disrupt the human CCR5 gene (3-6), which encodes a G-protein-coupled chemokine receptor, an essential co-receptor of HIV infection. A CCR5-specific ZFN is now under clinical investigation in the US for the treatment of AIDS (7). These ZFNs and TALENs, however, have off-target effects, inducing both local mutations at sites whose sequences are homologous to the on-target sequence (6, 8-10) and genome rearrangements that arise from the repair of two concurrent DSBs induced at on-target and off-target sites (11-12). The most striking off-target sites associated with these CCR5-specific engineered nucleases reside in the CCR2 locus, a close homolog of CCR5, located 15-kbp upstream of CCR5. To avoid off-target mutations in the CCR2 gene and unwanted deletions, inversions, and duplications of the 15-kbp chromosomal segment between the CCR5 on-target and CCR2 off-target sites, the present inventors intentionally chose the target site of our CCR5-specific RGEN to recognize a region within the CCR5 sequence that has no apparent homology with the CCR2 sequence.

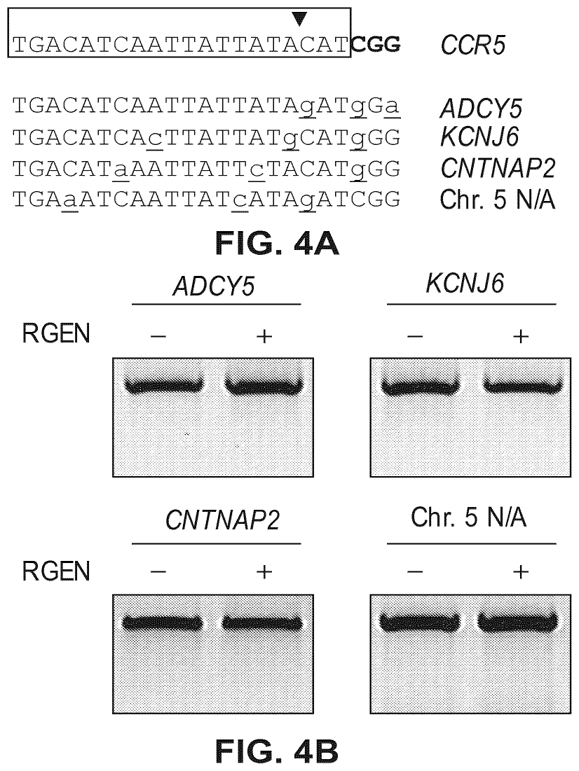

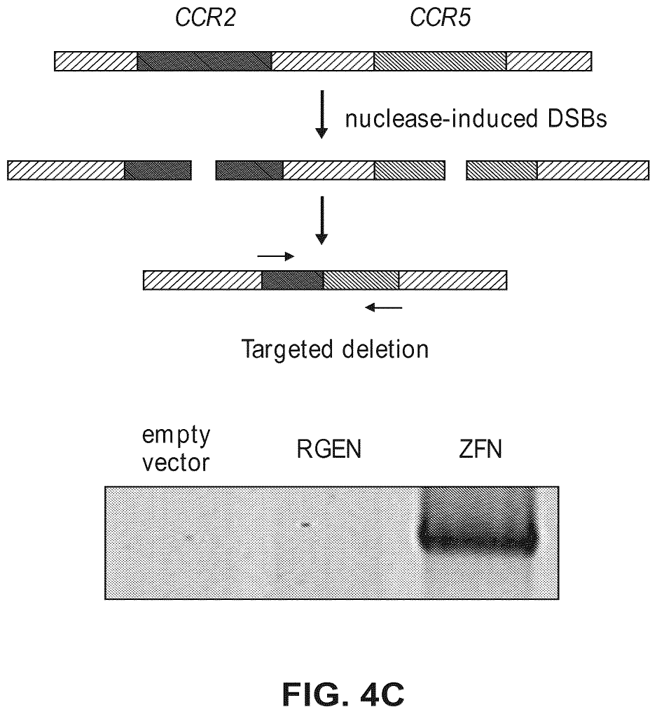

The present inventors investigated whether the CCR5-specific RGEN had off-target effects. To this end, we searched for potential off-target sites in the human genome by identifying sites that are most homologous to the intended 23-bp target sequence. As expected, no such sites were found in the CCR2 gene. Instead, four sites, each of which carries 3-base mismatches with the on-target site, were found (FIG. 4A). The T7E1 assays showed that mutations were not detected at these sites (assay sensitivity, ~0.5%), demonstrating exquisite specificities of RGENs (FIG. 4B). Furthermore, PCR was used to detect the induction of chromosomal deletions in cells separately transfected with plasmids encoding the ZFN and RGEN specific to CCR5. Whereas the ZFN induced deletions, the RGEN did not (FIG. 4C).

Next, RGENs was reprogrammed by replacing the CCR5-specific guide RNA with a newly-synthesized RNA designed to target the human C4BPB gene, which encodes the beta chain of C4b-binding protein, a transcription factor. This RGEN induced mutations at the chromosomal target site in K562 cells at high frequencies (FIG. 3B). Mutation frequencies measured by the T7E1 assay and by direct sequencing were 14% and 8.3% (=4 mutant clones/48 clones), respectively. Out of four mutant sequences, two clones contained a single-base or two-base insertion precisely at the cleavage site, a pattern that was also observed at the CCR5 target site. These results indicate that RGENs cleave chromosomal target DNA at expected positions in cells.

### Example 2: Proteinaceous RGEN-Mediated Genome Editing

RGENs can be delivered into cells in many different forms. RGENs consist of Cas9 protein, crRNA, and tracrRNA. The two RNAs can be fused to form a single-chain guide RNA (sgRNA). A plasmid that encodes Cas9 under a promoter such as CMV or CAG can be transfected into cells. crRNA, tracrRNA, or sgRNA can also be expressed in cells using plasmids that encode these RNAs. Use of plasmids, however, often results in integration of the whole or part of the plasmids in the host genome. The bacterial sequences incorporated in plasmid DNA can cause unwanted immune response in vivo. Cells transfected with plasmid for cell therapy or animals and plants derived from DNA-transfected cells must go through a costly and lengthy regulation procedure before market approval in most developed countries. Furthermore, plasmid DNA can persist in cells for several days post-transfection, aggravating off-target effects of RGENs.

Here, we used recombinant Cas9 protein complexed with in vitro transcribed guide RNA to induce targeted disruption of endogenous genes in human cells. Recombinant Cas9 protein fused with the hexa-histidine tag was expressed in and purified from E. coli using standard Ni ion affinity chromatography and gel filtration. Purified recombinant Cas9 protein was concentrated in storage buffer (20 mM HEPES pH 7.5, 150 mM KCl, 1 mM DTT, and 10% glycerol). Cas9 protein/sgRNA complex was introduced directly into K562 cells by nucleofection: 1×10⁶ K562 cells were transfected with 22.5-225 (1.4-14 μM) of Cas9 protein mixed with 100 ug (29 μM) of in vitro transcribed sgRNA (or crRNA 40 ug and tracrRNA 80 ug) in 100 μl solution using the 4D-Nucleofector, SF Cell Line 4D-Nucleofector X Kit, Program FF-120 (Lonza) according to the manufacturer's protocol. After nucleofection, cells were placed in growth media in 6-well plates and incubated for 48 hr. When 2×10⁵ K562 cells were transfected with ⅕ scale-downed protocol, 4.5-45 μg of Cas9 protein mixed with 6-60 ug of in vitro transcribed sgRNA (or crRNA 8 μg and tracrRNA 16 μg) were used and nucleofected in 20 μl solution. Nucleofected cells were then placed in growth media in 48-well plates. After 48 hr, cells were collected and genomic DNA was isolated. The genomic DNA region spanning the target site was PCR-amplified and subjected to the T7E1 assay.

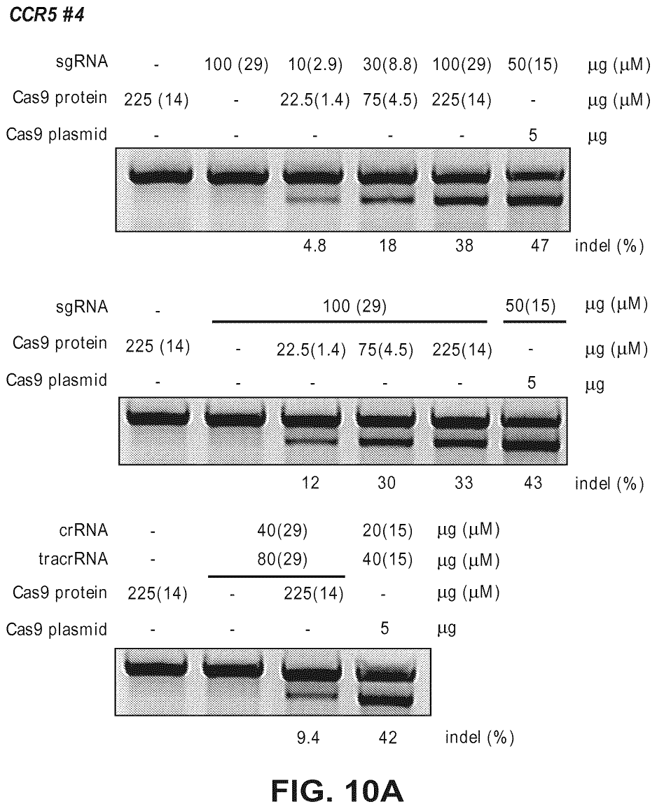

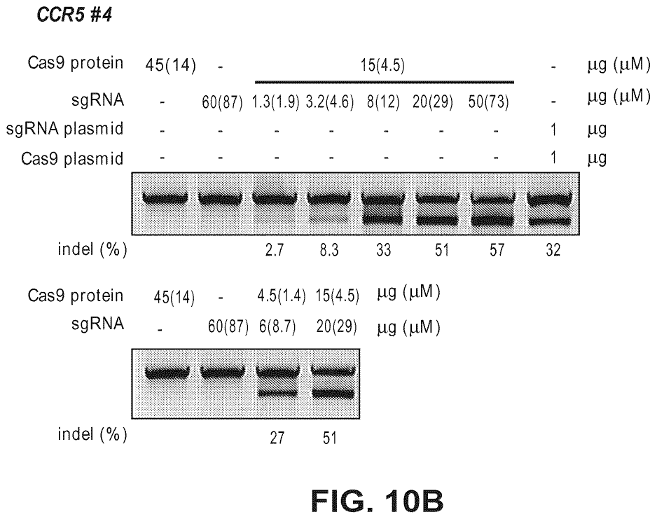

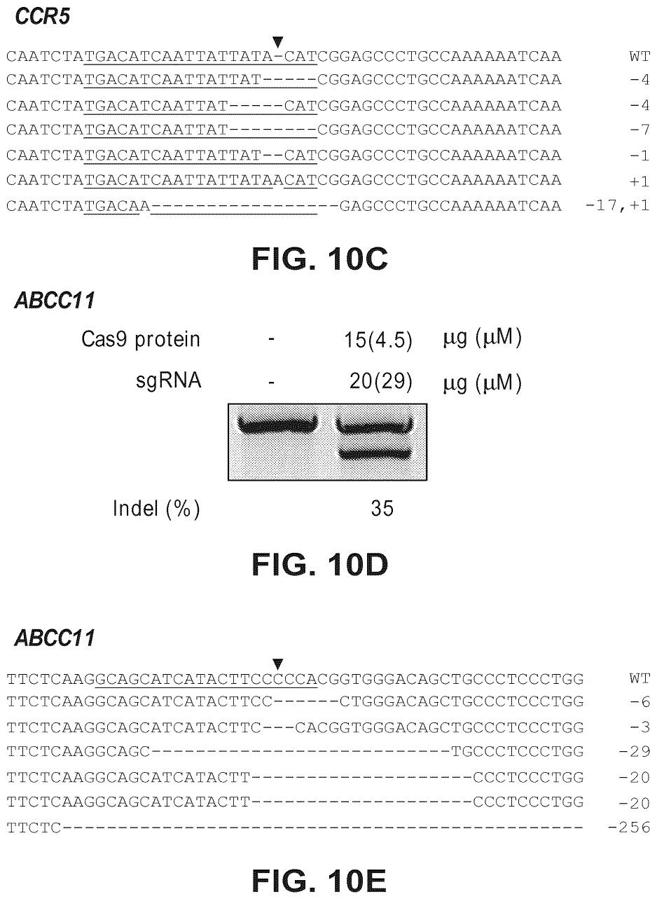

As shown in FIG. 10, Cas9 protein/sgRNA complex induced targeted mutation at the CCR5 locus at frequencies that ranged from 4.8 to 38% in a sgRNA or Cas9 protein dose-dependent manner, on par with the frequency obtained with Cas9 plasmid transfection (45%). Cas9 protein/crRNA/tracrRNA complex was able to induce mutations at a frequency of 9.4%. Cas9 protein alone failed to induce mutations. When 2×10⁵ cells were transfected with ⅕ scale-downed doses of Cas9 protein and sgRNA, mutation frequencies at the CCR5 locus ranged from 2.7 to 57% in a dose-dependent manner, greater than that obtained with co-transfection of Cas9 plasmid and sgRNA plasmid (32%).

We also tested Cas9 protein/sgRNA complex that targets the ABCC11 gene and found that this complex induced indels at a frequency of 35%, demonstrating general utility of this method.

TABLE 2
Sequences of guide RNA

| Target | RNA type | RNA sequence (5′ to 3′) | Length | SEQ ID NO |
|---|---|---|---|---|
| CCR5 | sgRNA | GGUGACAUCAAUUAUUAUACAUGUUUUAGAGCUAG | 104 bp | 28 |
| | | AAAUAGCAAGUUAAAAUAAGGCUAGUCCGUUAUCA | | |
| | | ACUUGAAAAAGUGGCACCGAGUCGGUGCUUUUUUU | | |
| | crRNA | GGUGACAUCAAUUAUUAUACAUGUUUUAGAGCUAU | 44 bp | 29 |
| | | GCUGUUUUG | | |
| | tracrRNA | GGAACCAUUCAAAACAGCAUAGCAAGUUAAAAUAA | 86 bp | 30 |
| | | GGCUAGUCCGUUAUCAACUUGAAAAAGUGGCACCG | | |
| | | AGUCGGUGCUUUUUUU | | |

### Example 3: RNA-Guided Genome Editing in Mice

To examine the gene-targeting potential of RGENs in pronuclear (PN)-stage mouse embryos, the forkhead box N1 (Foxn1) gene, which is important for thymus development and keratinocyte differentiation (Nehls et al., 1996), and the protein kinase, DNA activated, catalytic polypeptide (Prkdc) gene, which encodes an enzyme critical for DNA DSB repair and recombination (Taccioli et al., 1998) were used.

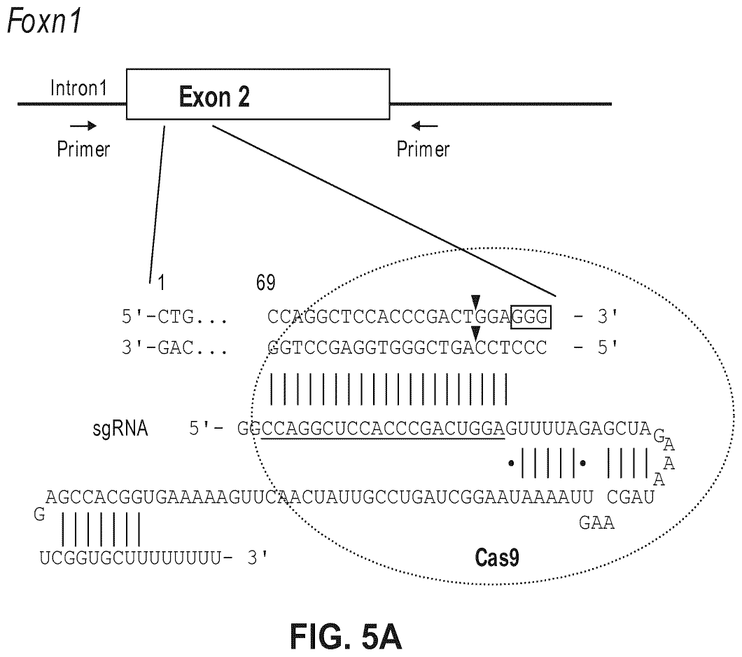

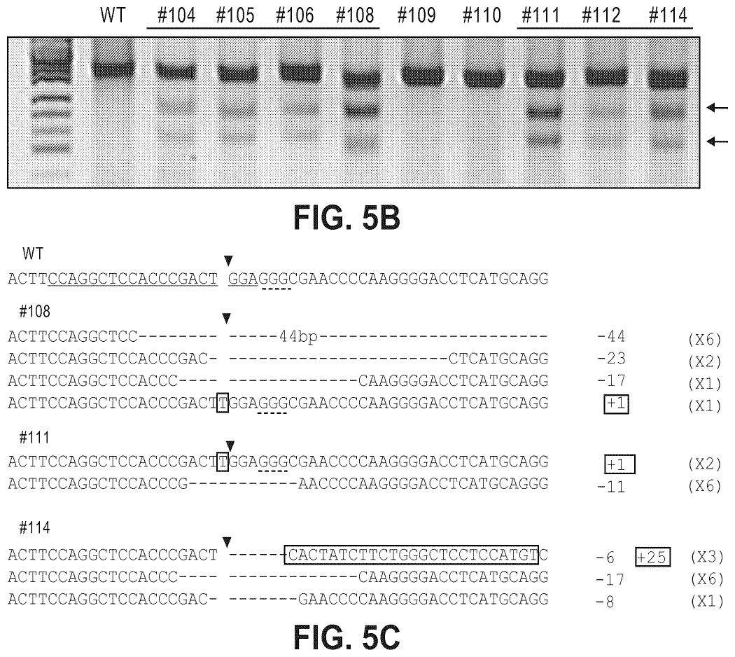

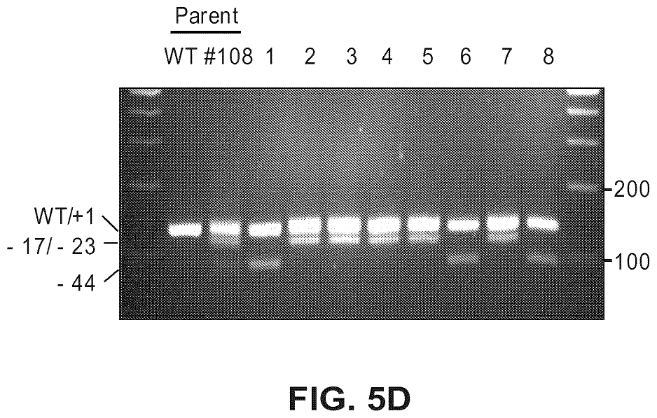

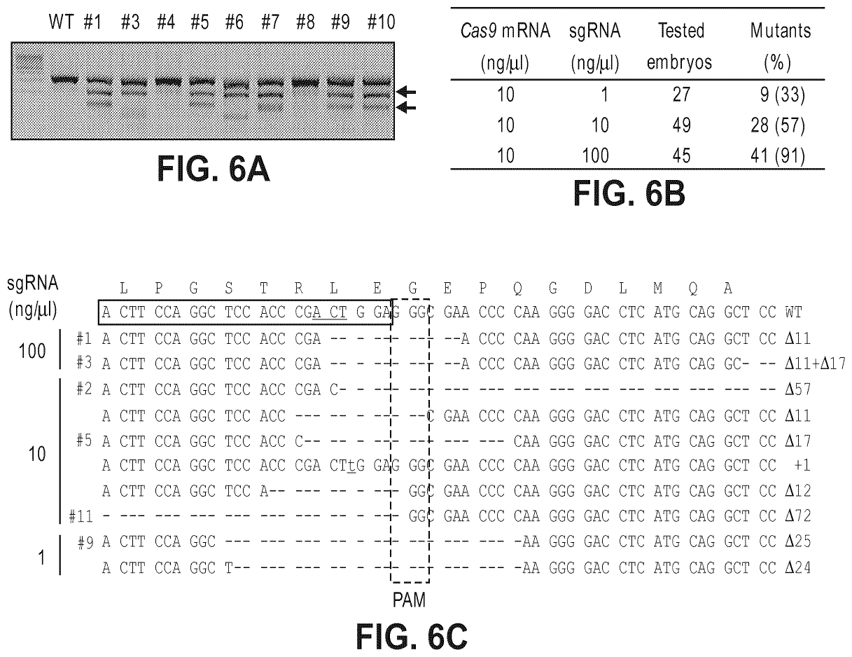

To evaluate the genome-editing activity of the Foxn1-RGEN, we injected Cas9 mRNA (10-ng/μl solution) with various doses of the sgRNA (FIG. 5a) into the cytoplasm of PN-stage mouse embryos, and conducted T7 endonuclease I (T7E1) assays (Kim et al. 2009) using genomic DNAs obtained from in vitro cultivated embryos (FIG. 6a).

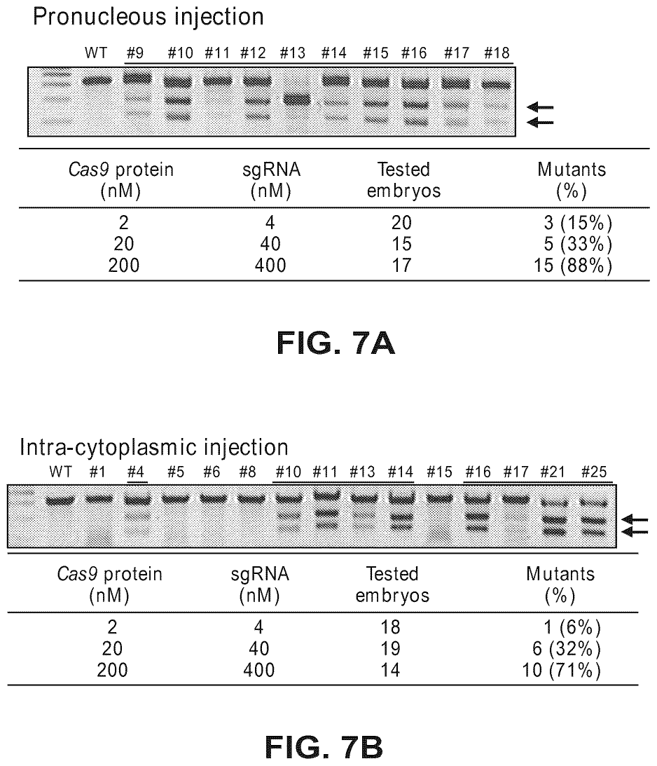

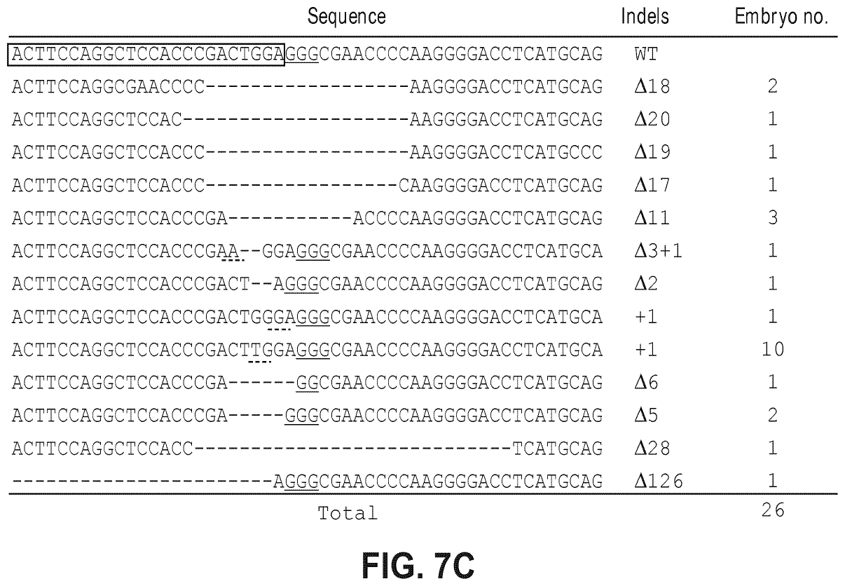

Alternatively, we directly injected the RGEN in the form of recombinant Cas9 protein (0.3 to 30 ng/μl) complexed with the two-fold molar excess of Foxn1-specific sgRNA (0.14 to 14 ng/μl) into the cytoplasm or pronucleus of one-cell mouse embryos, and analyzed mutations in the Foxn1 gene using in vitro cultivated embryos (FIG. 7).

Specifically, Cas9 mRNA and sgRNAs were synthesized in vitro from linear DNA templates using the mMESSAGE mMACHINE T7 Ultra kit (Ambion) and MEGAshortscript T7 kit (Ambion), respectively, according to the manufacturers' instructions, and were diluted with appropriate amounts of diethyl pyrocarbonate (DEPC, Sigma)-treated injection buffer (0.25 mM EDTA, 10 mM Tris, pH 7.4). Templates for sgRNA synthesis were generated using oligonucleotides listed in Table 3. Recombinant Cas9 protein was obtained from ToolGen, Inc.

TABLE 3

| RNA Name | Direction | Sequence (5′ to 3′) | SEQ ID NO |
|---|---|---|---|
| Foxn1 #1 | F | GAAATTAATACGACTCACTATAGGCAGTCTGACG | 31 |
| sgRNA | | TCACACTTCCGTTTTAGAGCTAGAAATAGCAAGT | |
| | | TAAAATAAGGCTAGTCCG | |
| Foxn1 #2 | F | GAAATTAATACGACTCACTATAGGACTTCCAGGC | 32 |
| sgRNA | | TCCACCCGACGTTTTAGAGCTAGAAATAGCAAGT | |
| | | TAAAATAAGGCTAGTCCG | |
| Foxn1 #3 | F | GAAATTAATACGACTCACTATAGGCCAGGCTCCA | 33 |
| sgRNA | | CCCGACTGGAGTTTTAGAGCTAGAAATAGCAAGT | |
| | | TAAAATAAGGCTAGTCCG | |
| Foxn1 #4 | F | GAAATTAATACGACTCACTATAGGACTGGAGGGC | 34 |
| sgRNA | | GAACCCCAAGGTTTTAGAGCTAGAAATAGCAAGT | |
| | | TAAAATAAGGCTAGTCCG | |
| Foxn1 #5 | F | GAAATTAATACGACTCACTATAGGACCCCAAGGG | 35 |
| sgRNA | | GACCTCATGCGTTTTAGAGCTAGAAATAGCAAGT | |
| | | TAAAATAAGGCTAGTCCG | |
| Prkdc #1 | F | GAAATTAATACGACTCACTATAGGTTAGTTTTTT | 36 |
| sgRNA | | CCAGAGACTTGTTTTAGAGCTAGAAATAGCAAGT | |
| | | TAAAATAAGGCTAGTCCG | |
| Prkdc #2 | F | GAAATTAATACGACTCACTATAGGTTGGTTTGCT | 37 |
| sgRNA | | TGTGTTTATCGTTTTAGAGCTAGAAATAGCAAGT | |
| | | TAAAATAAGGCTAGTCCG | |
| Prkdc #3 | F | GAAATTAATACGACTCACTATAGGCACAAGCAAA | 38 |
| sgRNA | | CCAAAGTCTCGTTTTAGAGCTAGAAATAGCAAGT | |
| | | TAAAATAAGGCTAGTCCG | |
| Prkdc #4 | F | GAAATTAATACGACTCACTATAGGCCTCAATGCT | 39 |
| sgRNA | | AAGCGACTTCGTTTTAGAGCTAGAAATAGCAAGT | |
| | | TAAAATAAGGCTAGTCCG | |

All animal experiments were performed in accordance with the Korean Food and Drug Administration (KFDA) guidelines. Protocols were reviewed and approved by the Institutional Animal Care and Use Committees (IACUC) of the Laboratory Animal Research Center at Yonsei University (Permit Number: 2013-0099). All mice were maintained in the specific pathogen-free facility of the Yonsei Laboratory Animal Research Center. FVB/NTac (Taconic) and ICR mouse strains were used as embryo donors and foster mothers, respectively. Female FVB/NTac mice (7-8 weeks old) were super-ovulated by intra-peritoneal injections of 5 IU pregnant mare serum gonadotropin (PMSG, Sigma) and 5 IU human chorionic gonadotropin (hCG, Sigma) at 48-hour intervals. The super-ovulated female mice were mated to FVB/NTac stud males, and fertilized embryos were collected from oviducts.

Cas9 mRNA and sgRNAs in M2 medium (Sigma) were injected into the cytoplasm of fertilized eggs with well-recognized pronuclei using a Piezo-driven micromanipulator (Prime Tech).

In the case of injection of recombinant Cas9 protein, the recombinant Cas9 protein: Foxn1-sgRNA complex was diluted with DEPC-treated injection buffer (0.25 mM EDTA, 10 mM Tris, pH 7.4) and injected into male pronuclei using a TransferMan NK2 micromanipulator and a FemtoJet® microinjector (Eppendorf).

The manipulated embryos were transferred into the oviducts of pseudopregnant foster mothers to produce live animals, or were cultivated in vitro for further analyses.

To screen F0 mice and in vitro cultivated mouse embryos with RGEN-induced mutations, T7E1 assays were performed as previously described using genomic DNA samples from tail biopsies and lysates of whole embryos (Cho et al., 2013).

Briefly, the genomic region encompassing the RGEN target site was PCR-amplified, melted, and re-annealed to form heteroduplex DNA, which was treated with T7 endonuclease 1 (New England Biolabs), and then analyzed by agarose gel electrophoresis. Potential off-target sites were identified by searching with bowtie 0.12.9 and were also similarly monitored by T7E1 assays. The primer pairs used in these assays were listed in Tables 4 and 5.

TABLE 4
Primers used in the T7E1 assay

| | Di- | | SEQ |
|---|---|---|---|
| | rec- | | ID |
| Gene | tion | Sequence(5′ to 3′) | NO |
| Foxn1 | F1 | GTCTGTCTATCATCTCTTCCCTTCTCTCC | 40 |
| | F2 | TCCCTAATCCGATGGCTAGCTCCAG | 41 |
| | R1 | ACGAGCAGCTGAAGTTAGCATGC | 42 |
| | R2 | CTACTCAATGCTCTTAGAGCTACCAGGCTTGC | 43 |
| Prkdc | F | GACTGTTGTGGGGAGGGCCG | 44 |
| | F2 | GGGAGGGCCGAAAGTCTTATTTTG | 45 |
| | R1 | CCTGAAGACTGAAGTTGGCAGAAGTGAG | 46 |
| | R2 | CTTTAGGGCTTCTTCTCTACAATCACG | 47 |

TABLE 5
Primers used for amplification of off-target sites

| Gene | Notation | Direction | Sequence(5′ to 3′) | SEQ ID NO |
|---|---|---|---|---|
| Foxn1 | off 1 | F | CTCGGTGTGTAGCCCTGAC | 48 |
| | | R | AGACTGGCCTGGAACTCACAG | 49 |
| | off 2 | F | CACTAAAGCCTGTCAGGAAGCCG | 50 |
| | | R | CTGTGGAGAGCACACAGCAGC | 51 |
| | off 3 | F | GCTGCGACCTGAGACCATG | 52 |
| | | R | CTTCAATGGCTTCCTGCTTAGGCTAC | 53 |
| | off 4 | F | GGTTCAGATGAGGCCATCCTTTC | 54 |
| | | R | CCTGATCTGCAGGCTTAACCCTTG | 55 |
| Prkdc | off 1 | F | CTCACCTGCACATCACATGTGG | 56 |
| | | R | GGCATCCACCCTATGGGGTC | 57 |
| | off 2 | F | GCCTTGACCTAGAGCTTAAAGAGCC | 58 |
| | | R | GGTCTTGTTAGCAGGAAGGACACTG | 59 |
| | off 3 | F | AAAACTCTGCTTGATGGGATATGTGGG | 60 |
| | | R | CTCTCACTGGTTATCTGTGCTCCTTC | 61 |
| | off 4 | F | GGATCAATAGGTGGTGGGGGATG | 62 |
| | | R | GTGAATGACACAATGTGACAGCTTCAG | 63 |
| | off 5 | F | CACAAGACAGACCTCTCAACATTCAGTC | 64 |
| | | R | GTGCATGCATATAATCCATTCTGATTGCTCTC | 65 |
| | off 6 | F1 | GGGAGGCAGAGGCAGGT | 66 |
| | | F2 | GGATCTCTGTGAGTTTGAGGCCA | 67 |
| | | R1 | GCTCCAGAACTCACTCTTAGGCTC | 68 |

Mutant founders identified by the T7E1 assay were further analyzed by fPCR. Appropriate regions of genomic DNA were sequenced as described previously (Sung et al., 2013). For routine PCR genotyping of F1 progenies, the following primer pairs were used for both wild-type and mutant alleles:

(SEQ ID NO: 69)
5′-CTACTCCCTCCGCAGTCTGA-3′
and
(SEQ ID NO: 70)
5′-CCAGGCCTAGGTTCCAGGTA-3′
for the Foxn1 gene,
(SEQ ID NO: 71)
5′-CCCCAGCATTGCAGATTTCC-3′
and
(SEQ ID NO: 72)
5′-AGGGCTTCTTCTCTACAATCACG-3′
for Prkdc gene.

In the case of injection of Cas9 mRNA, mutant fractions (the number of mutant embryos/the number of total embryos) were dose-dependent, ranging from 33% (1 ng/μl sgRNA) to 91% (100 ng/μl) (FIG. 6b). Sequence analysis confirmed mutations in the Foxn1 gene; most mutations were small deletions (FIG. 6c), reminiscent of those induced by ZFNs and TALENs (Kim et al., 2013).

In the case of injection of Cas9 protein, these injection doses and methods minimally affected the survival and development of mouse embryos in vitro: over 70% of RGEN-injected embryos hatched out normally in both experiments. Again, mutant fractions obtained with Cas9 protein injection were dose-dependent, and reached up to 88% at the highest dose via pronucleus injection and to 71% via intra-cytoplasmic injection (FIGS. 7a and 7b). Similar to the mutation patterns induced by Cas9 mRNA plus sgRNA (FIG. 6c), those induced by the Cas9 protein-sgRNA complex were mostly small deletions (FIG. 7c). These results clearly demonstrate that RGENs have high gene-targeting activity in mouse embryos.

Encouraged by the high mutant frequencies and low cytotoxicity induced by RGENs, we produced live animals by transferring the mouse embryos into the oviducts of pseudo-pregnant foster mothers.

Notably, the birth rates were very high, ranging from 58% to 73%, and were not affected by the increasing doses of Foxn1-sgRNA (Table 6).

TABLE 6
RGEN-mediated gene-targeting in FVB/NTac mice

| | Cas9 mRNA + | | Transferred | | Total | Live | |
|---|---|---|---|---|---|---|---|
| Target | sgRNA | Injected | embryos | | newborns | newborns | Founders |
| Gene | (ng/μl) | embryos | (%) | | (%) | * (%) | † (%) |
| Foxn1 | 10 + 1 | 76 | 62 | (82) | 45 (73) | 31 (50) | 12 (39) |
| | 10 + 10 | 104 | 90 | (87) | 52 (58) | 58 (64) | 33 (57) |
| | 10 + 100 | 100 | 90 | (90) | 62 (69) | 58 (64) | 54 (93) |
| | Total | 280 | 242 | (86) | 159 (66) | 147 (61) | 99 (67) |
| Prkdc | 50 + 50 | 73 | 58 | (79) | 35 (60) | 33 (57) | 11 (33) |
| | 50 + 100 | 79 | 59 | (75) | 22 (37) | 21 (36) | 7 (33) |
| | 50 + 250 | 94 | 73 | (78) | 37 (51) | 37 (51) | 21 (57) |
| | Total | 246 | 190 | (77) | 94 (49) | 91 (48) | 39 (43) |

Out of 147 newborns, we obtained 99 mutant founder mice. Consistent with the results observed in cultivated embryos (FIG. 6b), mutant fractions were proportional to the doses of Foxn1-sgRNA, and reached up to 93% (100 ng/μl Foxn1-sgRNA) (Tables 6 and 7, FIG. 5b).

TABLE 7
DNA sequences of Foxn1 mutant alleles identified from a subset
of T7E1-positive mutant founders

| | | | |
|---|---|---|---|
| ACTTCCAGGCTCCACCCGACTGGAGGGCGAACCCCAAGGGGACCTCATGCAGG | del + ins | # | Founder |
| (SEQ ID NO: 134) | | | mice |
| ACTTCCAGGC-------------------AACCCCAAGGGGACCTCATGCAGG | Δ19 | 1 | 20 |
| (SEQ ID NO: 297) | | | |
| ACTTCCAGGC------------------GAACCCCAAGGGGACCTCATGCAGG | Δ18 | 1 | 115 |
| (SEQ ID NO: 298) | | | |
| ACTTCCAGGCTCC---------------------------------------- | Δ60 | 1 | 19 |
| (SEQ ID NO: 299) | | | |
| ACTTCCAGGCTCC---------------------------------------- | Δ44 | 1 | 108 |
| (SEQ ID NO: 300) | | | |
| ACTTCCAGGCTCC---------------------CAAGGGGACCTCATGCAGG | Δ21 | 1 | 64 |
| (SEQ ID NO: 301) | | | |
| ACTTCCAGGCTCC------------TTAGGAGGCGAACCCCAAGGGGACCTCA | Δ12 + 6 | 1 | 126 |
| (SEQ ID NO: 302) | | | |
| ACTTCCAGGCTCCACC----------------------------TCATGCAGG | Δ28 | 1 | 5 |
| (SEQ ID NO: 303) | | | |
| ACTTCCAGGCTCCACCC---------------------CCAAGGGACCTCATG | Δ21 + 4 | 1 | 61 |
| (SEQ ID NO: 304) | | | |
| ACTTCCAGGCTCCACCC------------------AAGGGGACCTCATGCAGG | Δ18 | 2 | 95, 29 |
| (SEQ ID NO: 305) | | | |
| ACTTCCAGGCTCCACCC-----------------CAAGGGGACCTCATGCAGG | Δ17 | 7 | 12, 14, |
| (SEQ ID NO: 306) | | | 27, 66, |
| | | | 108, 114, |
| | | | 126 |
| ACTTCCAGGCTCCACCC---------------ACCCAAGGGGACCTCATGCAG | Δ15 + 1 | 1 | 32 |
| (SEQ ID NO: 307) | | | |
| ACTTCCAGGCTCCACCC---------------CACCCAAGGGGACCTCATGCA | Δ15 + 2 | 1 | 124 |
| (SEQ ID NO: 308) | | | |
| ACTTCCAGGCTCCACCC-------------ACCCCAAGGGGACCTCATGCAGG | Δ13 | 1 | 32 |
| (SEQ ID NO: 309) | | | |
| ACTTCCAGGCTCCACCC--------GGCGAACCCCAAGGGGACCTCATGCAGG | Δ8 | 1 | 110 |
| (SEQ ID NO: 310) | | | |
| ACTTCCAGGCTCCACCCT-------------------GGGGACCTCATGCAGG | Δ20 + 1 | 1 | 29 |
| (SEQ ID NO: 311) | | | |
| ACTTCCAGGCTCCACCCG-----------AACCCCAAGGGGACCTCATGCAGG | Δ11 | 1 | 111 |
| (SEQ ID NO: 312) | | | |
| ACTTCCAGGCTCCACCCGA----------------------ACCTCATGCAGG | Δ22 | 1 | 79 |
| (SEQ ID NO: 313) | | | |
| ACTTCCAGGCTCCACCCGA------------------GGGGACCTCATGCAGG | Δ18 | 2 | 13, 127 |
| (SEQ ID NO: 314) | | | |
| ACTTCCAGGCTCCACCCCA-----------------AGGGGACCTCATGCAGG | Δ17 | 1 | 24 |
| (SEQ ID NO: 315) | | | |
| ACTTCCAGGCTCCACCCGA-----------ACCCCAAGGGGACCTCATGCAGG | Δ11 | 5 | 14, 53, |
| (SEQ ID NO: 316) | | | 58, 69, |
| | | | 124 |
| ACTTCCAGGCTCCACCCGA----------GACCCCAAGGGGACCTCATGCAGG | Δ10 | 1 | 14 |
| (SEQ ID NO: 317) | | | |
| ACTTCCAGGCTCCACCCGA-----GGGCGAACCCCAAGGGGACCTCATGCAGG | Δ5 | 3 | 53, 79, |
| (SEQ ID NO: 318) | | | 115 |
| ACTTCCAGGCTCCACCCGAC-----------------------CTCATGCAGG | Δ23 | 1 | 108 |
| (SEQ ID NO: 319) | | | |
| ACTTCCAGGCTCCACCCGAC-----------CCCCAAGGGGACCTCATGCAGG | Δ11 | 1 | 3 |
| (SEQ ID NO: 320) | | | |
| ACTTCCAGGCTCCACCCGAC-----------GAAGGGCCCCAAGGGGACCTCA | Δ11 + 6 | 1 | 66 |
| (SEQ ID NO: 321) | | | |
| ACTTCCAGGCTCCACCCGAC--------GAACCCCAAGGGGACCTCATGCAGG | Δ8 | 2 | 3, 66 |
| (SEQ ID NO: 322) | | | |
| ACTTCCAGGCTCCACCCGAC-----GGCGAACCCCAAGGGGACCTCATGCAGG | Δ5 | 1 | 27 |
| (SEQ ID NO: 323) | | | |
| ACTTCCAGGCTCCACCCGAC--GTGCTTGAGGGCGAACCCCAAGGGGACCTCA | Δ2 + 6 | 2 | 5 |
| (SEQ ID NO: 324) | | | |
| ACTTCCAGGCTCCACCCGACT------CACTATCTTCTGGGCTCCTCCATGTC | Δ6 + 25 | 2 | 21, 114 |
| (SEQ ID NO: 325) | | | |
| ACTTCCAGGCTCCACCCGACT----TGGCGAACCCCAAGGGGACCTCATGCAG | Δ4 + 1 | 1 | 53 |
| (SEQ ID NO: 326) | | | |
| ACTTCCAGGCTCCACCCGACT--TGCAGGGCGAACCCCAAGGGGACCTCATGC | Δ2 + 3 | 1 | 126 |
| (SEQ ID NO: 327) | | | |
| ACTTCCAGGCTCCACCCGACTTGGAGGGCGAACCCCAAGGGGACCTCATGCAG | +1 | 15 | 3, 5, |
| (SEQ ID NO: 328) | | | 12, 19, |
| | | | 29, 55, |
| | | | 56, 61, |
| | | | 66, 68, |
| | | | 81, |
| | | | 108, 111, |
| | | | 124, 127 |
| ACTTCCAGGCTCCACCCGACTTTGGAGGGCGAACCCCAAGGGGACCTCATGCA | +2 | 2 | 79, 120 |
| (SEQ ID NO: 329) | | | |
| ACTTCCAGGCTCCACCCGACTGTTGGAGGGCGAACCCCAAGGGGACCTCATGC | +3 | 1 | 55 |
| (SEQ ID NO: 330) | | | |
| ACTTCCAGGCTCCACCCGACTGGAG(+455)GGCGAACCCCAAGGGGACCTCC | +455 | 1 | 13 |
| (SEQ ID NO: 331) | | | |

To generate Prkdc-targeted mice, we applied a 5-fold higher concentration of Cas9 mRNA (50 ng/μl) with increasing doses of Prkdc-sgRNA (50, 100, and 250 ng/μl). Again, the birth rates were very high, ranging from 51% to 600, enough to produce a sufficient number of newborns for the analysis (Table 6). The mutant fraction was 570 (21 mutant founders among 37 newborns) at the maximum dose of Prkdc-sgRNA. These birth rates obtained with RGENs were approximately 2- to 10-fold higher than those with TALENs reported in our previous study (Sung et al., 2013). These results demonstrate that RGENs are potent gene-targeting reagents with minimal toxicity.

To test the germ-line transmission of the mutant alleles, we crossed the Foxn1 mutant founder #108, a mosaic with four different alleles (FIG. 5c, and Table 8) with wild-type mice, and monitored the genotypes of F1 offspring.

TABLE 8
Genotypes of Foxn1 mutant mice

| | sgRNA | Genotyping | |
|---|---|---|---|
| Founder NO. | (ng/ml) | Summary | Detected alleles |
| 58* | 1 | not determined | Δ11 |
| 19 | 100 | bi-allelic | Δ60/+1 |
| 20 | 100 | bi-allelic | Δ67/Δ19 |
| 13 | 100 | bi-allelic | Δ18/+455 |
| 32 | 10 | bi-allelic | Δ13/Δ15+1 |
| | | (heterozygote) | |
| 115 | 10 | bi-allelic | Δ18/Δ5 |
| | | (heterozygote) | |
| 111 | 10 | bi-allelic | Δ11/+1 |
| | | (heterozygote) | |
| 110 | 10 | bi-allelic | Δ8/Δ8 |
| | | (homozygote) | |
| 120 | 10 | bi-allelic | +2/+2 |
| | | (homozygote) | |
| 81 | 100 | heterozygote | +1/WT |
| 69 | 100 | homozygote | Δ11/Δ11 |
| 55 | 1 | mosaic | Δ18/Δ1/+1/+3 |
| 56 | 1 | mosaic | Δ127/Δ41/Δ2/+1 |
| 127 | 1 | mosaic | Δ18/+1/WT |
| 53 | 1 | mosaic | Δ11/Δ5/Δ4+1/WT |
| 27 | 10 | mosaic | Δ17/Δ5/WT |
| 29 | 10 | mosaic | Δ18/Δ20+1/+1 |
| 95 | 10 | mosaic | Δ18/Δ14/Δ8/Δ4 |
| 108 | 10 | mosaic | +1/Δ17/Δ23/Δ44 |
| 114 | 10 | mosaic | Δ17/Δ8/Δ6+25 |
| 124 | 10 | mosaic | Δ11/Δ15+2/+1 |
| 126 | 10 | mosaic | Δ17/Δ2+3/Δ12+6 |
| 12 | 100 | mosaic | Δ30/Δ28/Δ17/+1 |
| 5 | 100 | mosaic | Δ28/Δ11/Δ2+6/+1 |
| 14 | 100 | mosaic | Δ17/Δ11/Δ10 |
| 21 | 100 | mosaic | Δ127/Δ41/Δ2/Δ6+25 |
| 24 | 100 | mosaic | Δ17/+1/WT |
| 64 | 100 | mosaic | Δ31/Δ21/+1/WT |
| 68 | 100 | mosaic | Δ17/Δ11/+1/WT |
| 79 | 100 | mosaic | Δ22/Δ5/+2/WT |
| 61 | 100 | mosaic | Δ21+4/Δ6/+1/+9 |
| 66** | 100 | mosaic | Δ17/Δ8/Δ11+6/+1/WT |
| 3 | 100 | mosaic | Δ11/Δ8/+1 |

Underlined alleles were sequenced.
Alleles in red, detected by sequencing, but not by fPCR.
*only one clone sequenced.
**Not determined by fPCR.

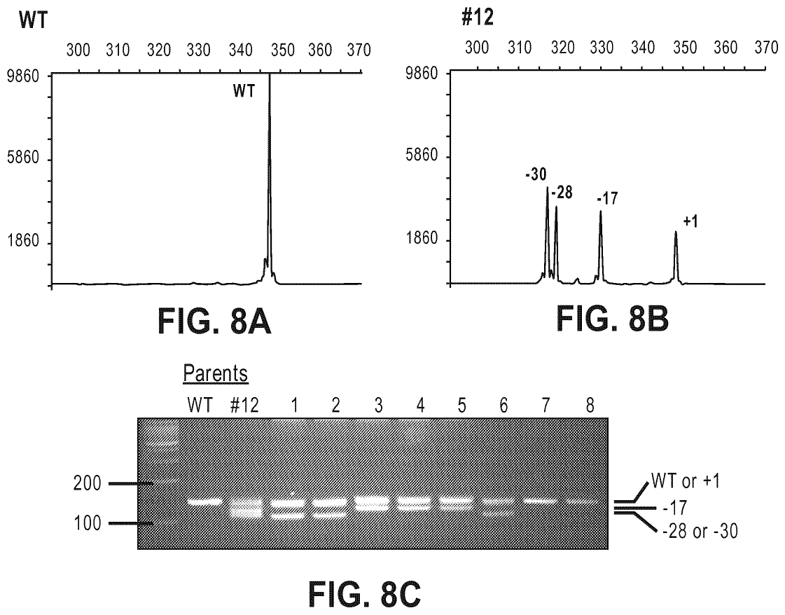

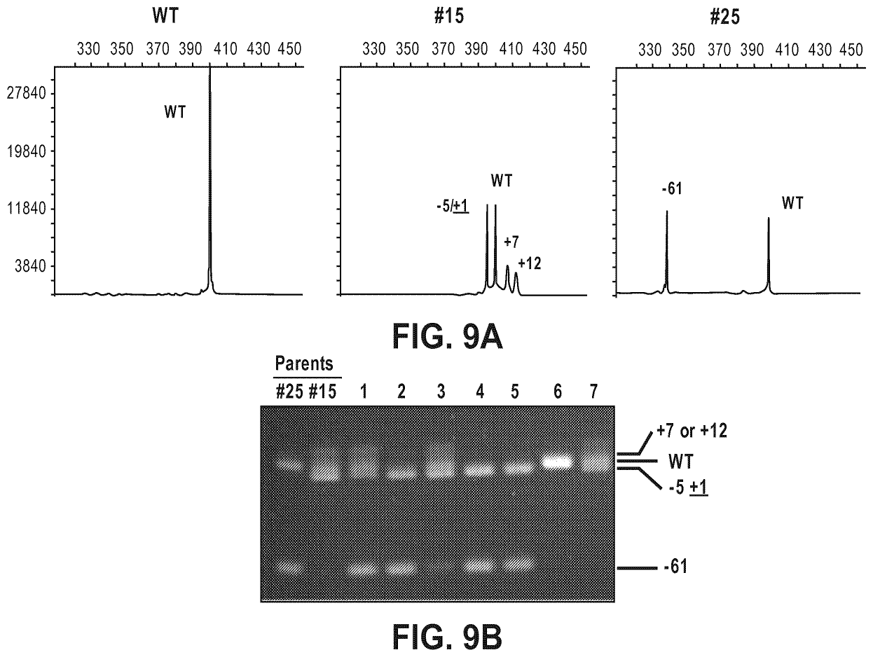

As expected, all the progenies were heterozygous mutants possessing the wild-type allele and one of the mutant alleles (FIG. 5d). We also confirmed the germ-line transmission in independent founder mice of Foxn1 (FIG. 8) and Prkdc (FIG. 9). To the best of our knowledge, these results provide the first evidence that RGEN-induced mutant alleles are stably transmitted to F1 progenies in animals.

### Example 4: RNA-Guided Genome Editing in Plants

### 4-1. Production of Cas9 Protein

The Cas9 coding sequence (4104 bps), derived from Streptococcus pyogenes strain M1 GAS (NC_002737.1), was cloned to pET28-b(+) plasmid. A nuclear targeting sequence (NLS) was included at the protein N terminus to ensure the localization of the protein to the nucleus. pET28-b(+) plasmid containing Cas9 ORF was transformed into BL21(DE3). Cas9 was then induced using 0.2 mM IPTG for 16 hrs at 18° C. and purified using Ni-NTA agarose beads (Qiagen) following the manufacturer's instructions. Purified Cas9 protein was concentrated using Ultracel—100K (Millipore).

### 4-2. Production of Guide RNA

The genomic sequence of the Arabidopsis gene encoding the BRI1 was screened for the presence of a NGG motif, the so called protospacer adjacent motif (PAM), in an exon which is required for Cas9 targeting To disrupt the BRI1 gene in Arabidopsis, we identified two RGEN target sites in an exon that contain the NGG motif. sgRNAs were produced in vitro using template DNA. Each template DNA was generated by extension with two partially overlapped oligonucleotides (Macrogen, Table X1) and Phusion polymerase (Thermo Scientific) using the following conditions—98° C. 30 sec {98° C. 10 sec, 54° C. 20 sec, 72° C. 2 min}×20, 72° C. 5 min.

TABLE 9
Oligonucleotides for the production of the
template DNA for in vitro transcription

| Oligonucleotides | Sequence (5′-3′) | SEQ ID NO |
|---|---|---|
| BRI1 target 1 | GAAATTAATACGACTCACTATAGGTTTGAAAGATGG | 73 |
| (Forward) | AAGCGCGGGTTTTAGAGCTAGAAATAGCAAGTTAAA | |
| | ATAAGGCTAGTCCG | |
| BRI1 target 2 | GAAATTAATACGACTCACTATAGGTGAAACTAAACT | 74 |
| (Forward) | GGTCCACAGTTTTAGAGCTAGAAATAGCAAGTTAAA | |
| | ATAAGGCTAGTCCG | |
| Universal | AAAAAAGCACCGACTCGGTGCCACTTTTTCAAGTTG | 75 |
| (Reverse) | ATAACGGACTAGCCTTATTTTAACTTGC | |

The extended DNA was purified and used as a template for the in vitro production of the guide RNA's using the MEGAshortscript T7 kit (Life Technologies). Guide RNA were then purified by Phenol/Chloroform extraction and ethanol precipitation. To prepare Cas9/sgRNA complexes, 10 μl of purified Cas9 protein (12 μg/μl) and 4 μl each of two sgRNAs (11 μg/μl) were mixed in 20 μl NEB3 buffer (New England Biolabs) and incubated for 10 min at 37° C.

### 4-3. Transfection of Cas9/sgRNA Complex to Protoplast

The leaves of 4-week-old Arabidopsis seedlings grown aseptically in petri dishes were digested in enzyme solution (1% cellulose R10, 0.5% macerozyme R10, 450 mM mannitol, 20 mM MES pH 5.7 and CPW salt) for 8~16 hrs at 25° C. with 40 rpm shaking in the dark. Enzyme/protoplast solutions were filtered and centrifuged at 100×g for 3~5 min. Protoplasts were re-suspended in CPW solution after counting cells under the microscope (×100) using a hemacytometer. Finally, protoplasts were re-suspended at 1×10⁶/ml in MMG solution (4 mM HEPES pH 5.7, 400 mM mannitol and 15 mM MgCl2). To transfect the protoplasts with Cas9/sgRNA complex, 200 μL (200,000 protoplasts) of the protoplast suspension were gently mixed with 3.3 or 10 μL of Cas9/sgRNA complex [Cas9 protein (6 μg/μL) and two sgRNAs (2.2 μg/μL each)] and 200 μL of 40% polyethylene glycol transfection buffer (40% PEG4000, 200 mM mannitol and 100 mM CaCl2)) in 2 ml tubes. After 5~20 min incubation at room temperature, transfection was stopped by adding wash buffer with W5 solution (2 mM MES pH 5.7, 154 mM NaCl, 125 mM CaCl2) and 5 mM KCl). Protoplasts were then collected by centrifugation for 5 min at 100×g, washed with 1 ml of W5 solution, centrifuged for another 5 min at 100×g. The density of protoplasts was adjusted to 1×10⁵/ml and they were cultured in modified KM 8p liquid medium with 400 mM glucose.

### 4-4. Detection of Mutations in Arabidopsis Protoplasts and Plants

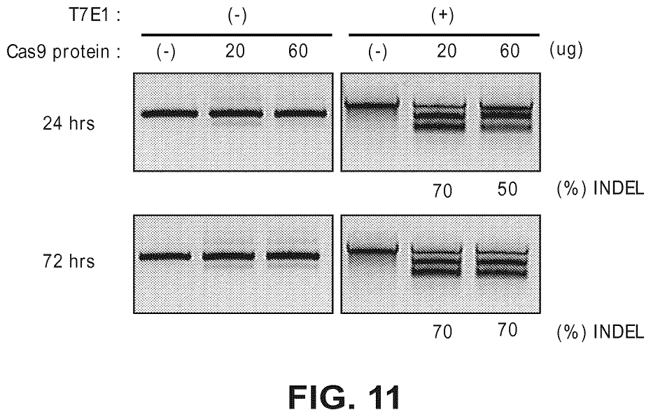

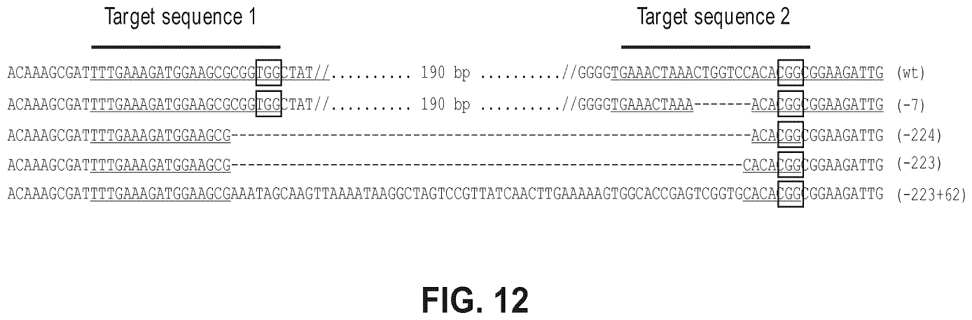

After 24 hr or 72 hr post-transfection, protoplasts were collected and genomic DNA was isolated. The genomic DNA region spanning the two target sites was PCR-amplified and subjected to the T7E1 assay. As shown in FIG. 11, indels were induced by RGENs at high frequencies that ranged from 50% to 70%. Surprisingly, mutations were induced at 24 hr post-transfection. Apparently, Cas9 protein functions immediately after transfection. PCR products were purified and cloned into T-Blunt PCR Cloning Kit (Solgent). Plasmids were purified and subjected to Sanger sequencing with M13F primer. One mutant sequence had a 7-bp deletion at one site (FIG. 12). The other three mutant sequences had deletions of ~220-bp DNA segments between the two RGEN site.

### Example 5: Cas9 Protein Transduction Using a Cell-Penetrating Peptide or Protein Transduction Domain

5-1. Construction of his-Cas9-Encoding Plasmid

Cas9 with a cysteine at the C-terminal was prepared by PCR amplification using the previously described Cas9 plasmid {Cho, 2013 #166} as the template and cloned into pET28-(a) vector (Novagen, Merk Millipore, Germany) containing His-tag at the N-terminus.

### 5-2. Cell Culture

293T (Human embryonic kidney cell line), and HeLa (human ovarian cancer cell line) were grown in DMEM (GIBCO-BRL Rockville) supplemented with 10% FBS and 1% penicillin and streptomycin.

### 5-3. Expression and Purification of Cas9 Protein

To express the Cas9 protein, E. coli BL21 cells were transformed with the pET28-(a) vector encoding Cas9 and plated onto Luria-Bertani (LB) agar medium containing 50 μg/mL kanamycin (Amresco, Solon, OH). Next day, a single colony was picked and cultured in LB broth containing 50 μg/mL kanamycin at 37° C. overnight. Following day, this starter culture at 0.1 OD600 was inoculated into Luria broth containing 50 μg/mL kanamycin and incubated for 2 hrs at 37° C. until OD600 reached to 0.6-0.8. To induce Cas9 protein expression, the cells were cultured at 30° C. overnight after addition of isopropyl-R-D-thiogalactopyranoside (IPTG) (Promega, Madison, WI) to the final concentration of 0.5 mM.

The cells were collected by centrifugation at 4000 rpm for 15-20 mins, resuspended in a lysis buffer (20 mM Tris-Cl pH8.0, 300 mM NaCl, 20 mM imidazole, 1× protease inhibitor cocktail, 1 mg/ml lysozyme), and lysed by sonication (40% duty, 10 sec pulse, 30 sec rest, for 10 mins on ice). The soluble fraction was separated as the supernatant after centrifugation at 15,000 rpm for 20 mins at 4° C. Cas9 protein was purified at 4° C. using a column containing Ni-NTA agarose resin (QIAGEN) and AKTA prime instrument (AKTA prime, GE Healthcare, UK). During this chromatography step, soluble protein fractions were loaded onto Ni-NTA agarose resin column (GE Healthcare, UK) at the flow rate of 1 mL/min. The column was washed with a washing buffer (20 mM Tris-Cl pH8.0, 300 mM NaCl, 20 mM imidazole, 1× protease inhibitor cocktail) and the bound protein was eluted at the flow rate of 0.5 ml/min with an elution buffer (20 mM Tris-Cl pH8.0, 300 mM NaCl, 250 mM imidazole, 1× protease inhibitor cocktail). The pooled eluted fraction was concentrated and dialyzed against storage buffer (50 mM Tris-HCl, pH8.0, 200 mM KCl, 0.1 mM EDTA, 1 mM DTT, 0.5 mM PMSF, 20% Glycerol). Protein concentration was quantitated by Bradford assay (Biorad, Hercules, CA) and purity was analyzed by SDS-PAGE using bovine serum albumin as the control.

### 5-4. Conjugation of Cas9 to 9R4L

1 mg Cas9 protein diluted in PBS at the concentration of 1 mg/mL and 50 μg of maleimide-9R4L peptide in 25 μL DW (Peptron, Korea) were gently mixed using a rotor at room temperature for 2 hrs and at 4° C. overnight. To remove unconjugated maleimide-9R4L, the samples were dialyzed using 50 kDa molecular weight cutoff membrane against of DPBS (pH 7.4) at 4° C. for 24 hrs. Cas9-9R4L protein was collected from the dialysis membrane and the protein amount was determined using Bradford assay.

5-5. Preparation of sgRNA-9R4L

sgRNA (1 μg) was gently added to various amounts of C9R4LC peptide (ranging from 1 to 40 weight ratio) in 100 μl of DPBS (pH 7.4). This mixture was incubated at room temperature for 30 mins and diluted to 10 folds using RNAse-free deionized water. The hydrodynamic diameter and z-potential of the formed nanoparticles were measured using dynamic light scattering (Zetasizer-nano analyzer ZS; Malvern instruments, Worcestershire, UK).

5-6. Cas9 Protein and sgRNA Treatments

Cas9-9R4L and sgRNA-C9R4LC were treated to the cells as follows: 1 μg of sgRNA and 15 μg of C9R4LC peptide were added to 250 mL of OPTIMEM medium and incubated at room temperature for 30 mins. At 24 hrs after seeding, cells were washed with OPTIMEM medium and treated with sgRNA-C9R4LC complex for 4 hrs at 37° C. Cells were washed again with OPTIMEM medium and treated with Cas9-9R4L for 2 hrs at 37° C. After treatment, culture media was replaced with serum-containing complete medium and incubated at 37° C. for 24 hrs before the next treatment. Same procedure was followed for multiple treatments of Cas9 and sgRNA for three consecutive days.

5-7. Cas9-9R4L and sgRNA-9R4L can Edit Endogenous Genes in Cultured Mammalian Cells without the Use of Additional Delivery Tools

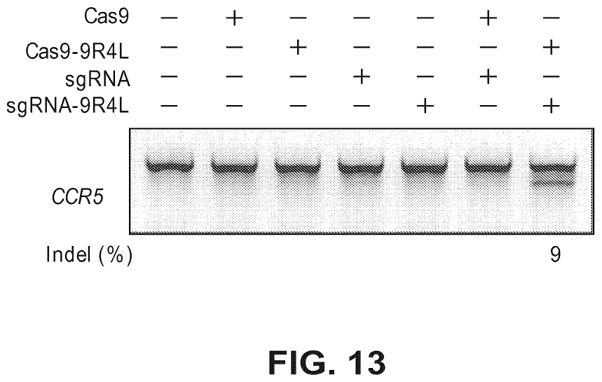

To determine whether Cas9-9R4L and sgRNA-9R4L can edit endogenous genes in cultured mammalian cells without the use of additional delivery tools, we treated 293 cells with Cas9-9R4L and sgRNA-9R4L targeting the CCR5 gene and analyzed the genomic DNA. T7E1 assay showed that 9% of CCR5 gene was disrupted in cells treated with both Cas9-9R4L and sgRNA-9R4L and that the CCR5 gene disruption was not observed in control cells including those untreated, treated with either Cas9-9R or sgRNA-9R4L, or treated with both unmodified Cas-9 and sgRNA (FIG. 13), suggesting that the treatment with Cas9-9R4L protein and sgRNA conjugated with 9R4L, but not unmodified Cas9 and sgRNA, can lead to efficient genome editing in mammalian cells.

### Example 6: Control of Off-Target Mutation According to Guide RNA Structure

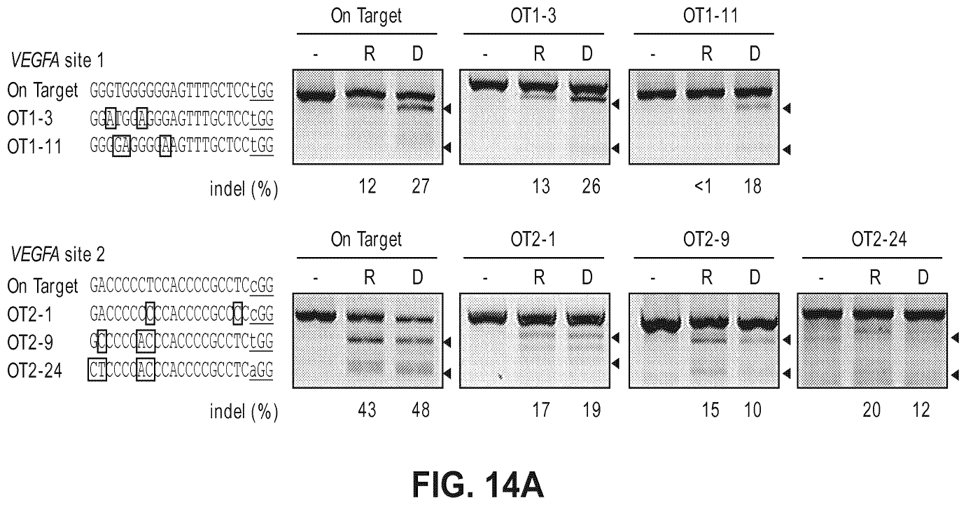

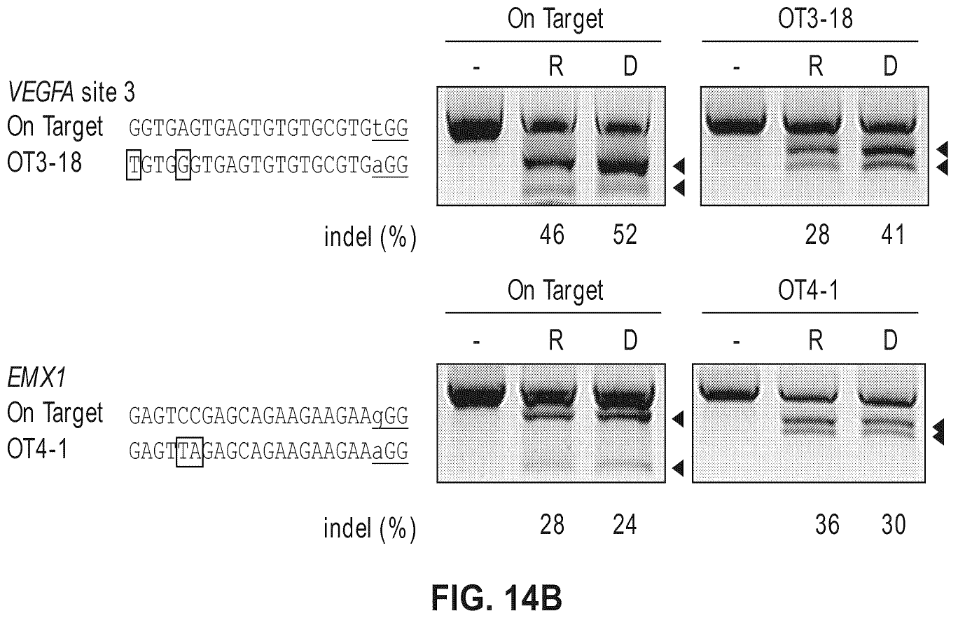

Recently, three groups reported that RGENs had off-target effects in human cells. To our surprise, RGENs induced mutations efficiently at off-target sites that differ by 3 to 5 nucleotides from on-target sites. We noticed, however, that there were several differences between our RGENs and those used by others. First, we used dualRNA, which is crRNA plus tracrRNA, rather than single-guide RNA (sgRNA) that is composed of essential portions of crRNA and tracrRNA. Second, we transfected K562 cells (but not HeLa cells) with synthetic crRNA rather than plasmids encoding crRNA. HeLa cells were transfected with crRNA-encoding plasmids. Other groups used sgRNA-encoding plasmids. Third, our guide RNA had two additional guanine nucleotides at the 5′ end, which are required for efficient transcription by T7 polymerase in vitro. No such additional nucleotides were included in the sgRNA used by others. Thus, the RNA sequence of our guide RNA can be shown as 5′-GGX₂₀, whereas 5′-GX₁₉, in which X₂₀ or GX₁₉ corresponds to the 20-bp target sequence, represents the sequence used by others. The first guanine nucleotide is required for transcription by RNA polymerase in cells. To test whether off-target RGEN effects can be attributed to these differences, we chose four RGENs that induced off-target mutations in human cells at high frequencies (13). First, we compared our method of using in vitro transcribed dualRNA with the method of transfecting sgRNA-encoding plasmids in K562 cells and measured mutation frequencies at the on-target and off-target sites via the T7E1 assay. Three RGENs showed comparable mutation frequencies at on-target and off-target sites regardless of the composition of guide RNA. Interestingly, one RGEN (VEFGA site 1) did not induce indels at one validated off-target site, which differs by three nucleotides from the on-target site (termed OT1-11, FIG. 14), when synthetic dualRNA was used. But the synthetic dualRNA did not discriminate the other validated off-target site (OT1-3), which differs by two nucleotides from the on-target site.

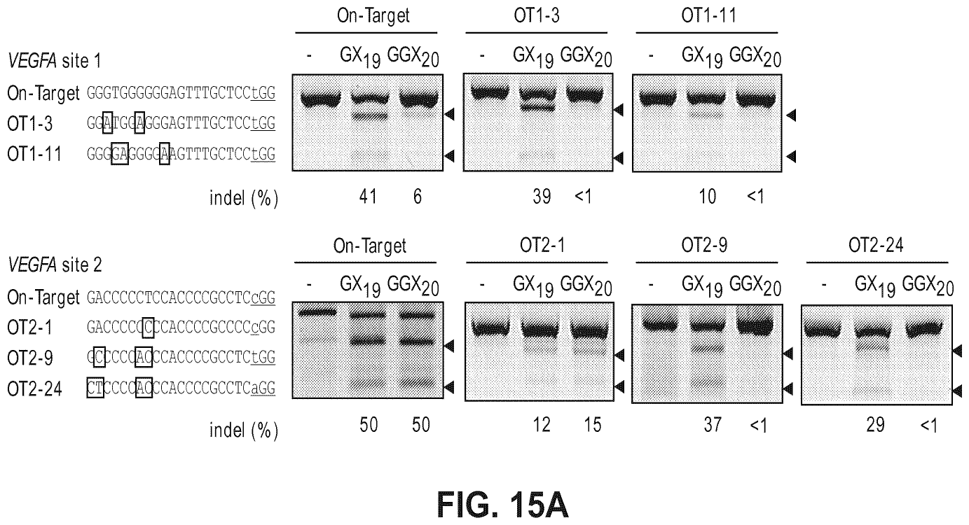

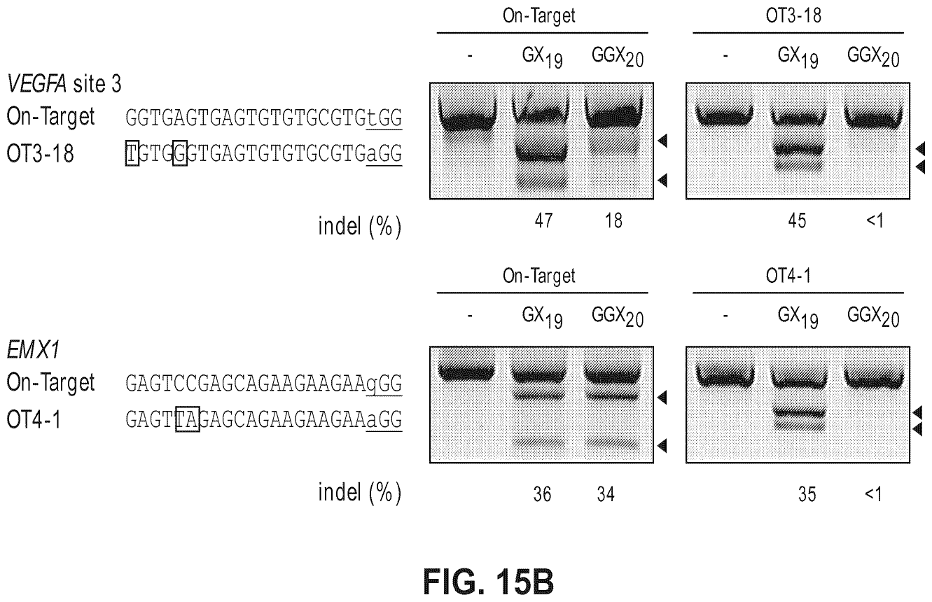

Next, we tested whether the addition of two guanine nucleotides at the 5′ end of sgRNA could make RGENs more specific by comparing 5′-GGX₂₀ (or 5′-GGGX₁₉) sgRNA with 5′-GX₁₉ sgRNA. Four GX₁₉ sgRNAs complexed with Cas9 induced indels equally efficiently at on-target and off-target sites, tolerating up to four nucleotide mismatches. In sharp contrast, GGX₂₀ sgRNAs discriminated off-target sites effectively. In fact, the T7E1 assay barely detected RGEN-induced indels at six out of the seven validated off-target sites when we used the four GGX₂₀ sgRNAs (FIG. 15). We noticed, however, that two GGX₂₀ sgRNAs (VEGFA sites 1 and 3) were less active at on-target sites than were the corresponding GX₁₉ sgRNAs. These results show that the extra nucleotides at the 5′ end can affect mutation frequencies at on-target and off-target sites, perhaps by altering guide RNA stability, concentration, or secondary structure.

These results suggest that three factors-the use of synthetic guide RNA rather than guide RNA-encoding plasmids, dualRNA rather than sgRNA, and GGX₂₀ sgRNA rather than GX₁₉ sgRNA-have cumulative effects on the discrimination of off-target sites.

### Example 7: Paired Cas9 Nickases

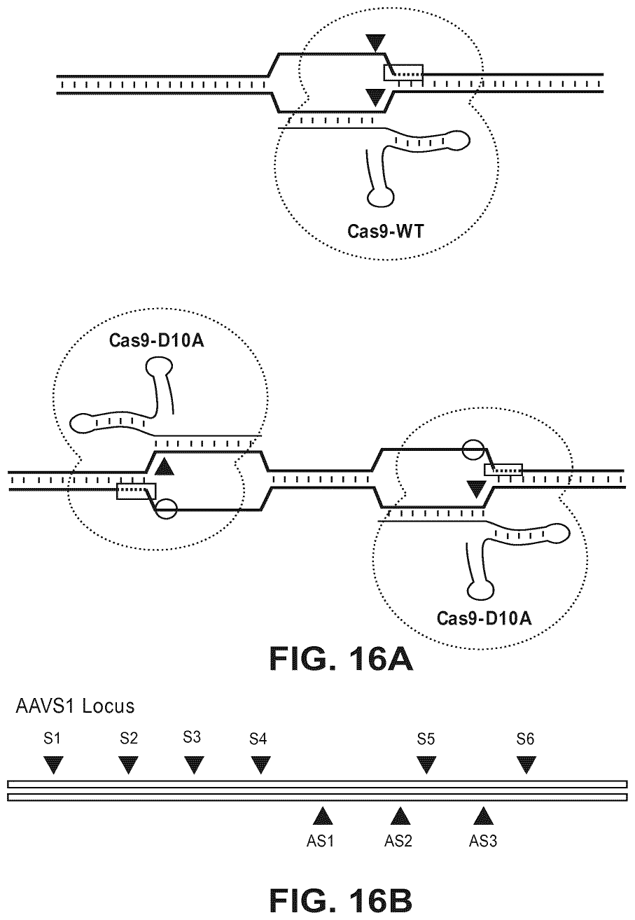

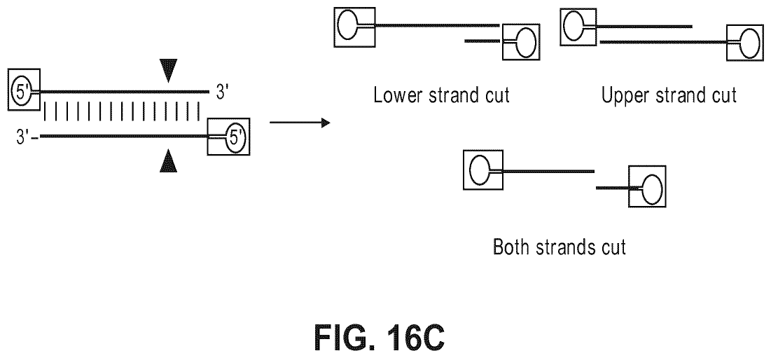

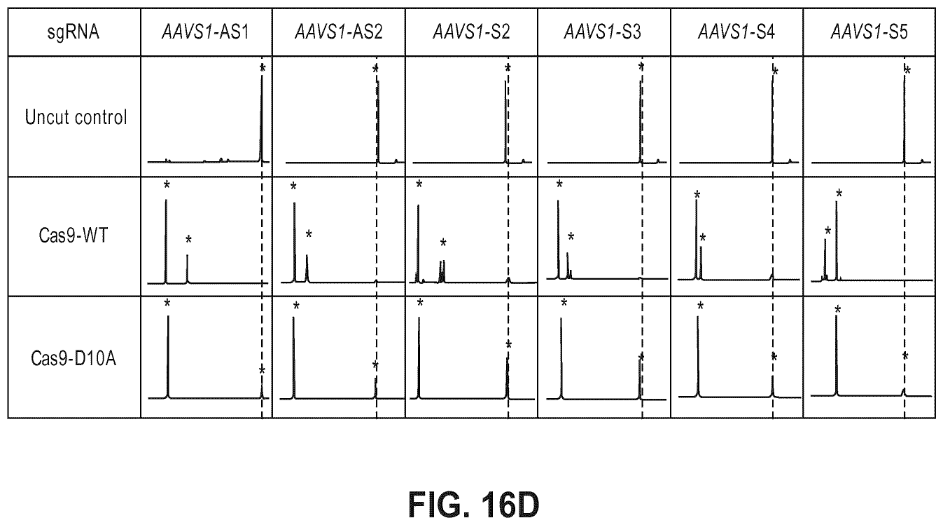

In principle, single-strand breaks (SSBs) cannot be repaired by error-prone NHEJ but still trigger high fidelity homology-directed repair (HDR) or base excision repair. But nickase-induced targeted mutagenesis via HDR is much less efficient than is nuclease-induced mutagenesis. We reasoned that paired Cas9 nickases would produce composite DSBs, which trigger DNA repair via NHEJ or HDR, leading to efficient mutagenesis (FIG. 16A). Furthermore, paired nickases would double the specificity of Cas9-based genome editing.

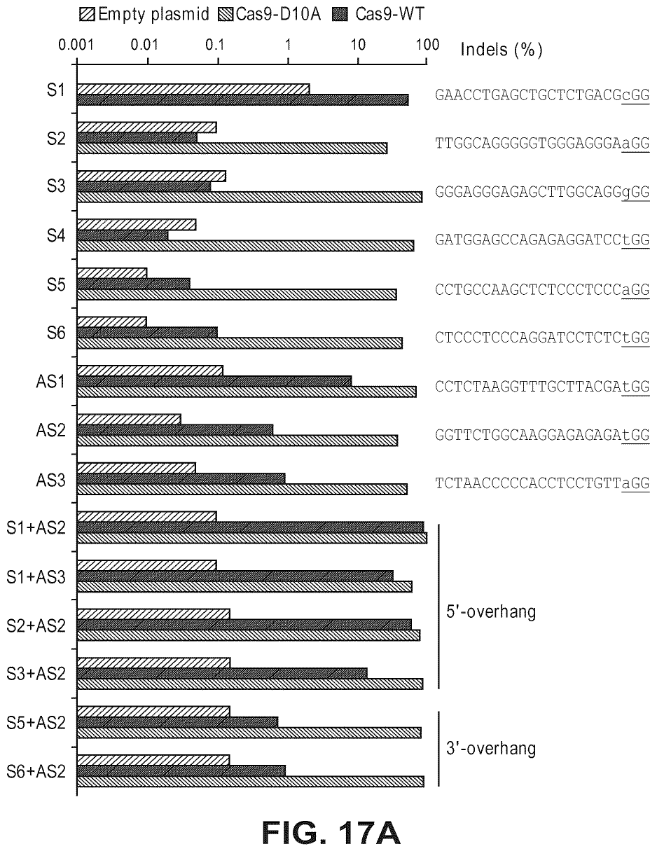

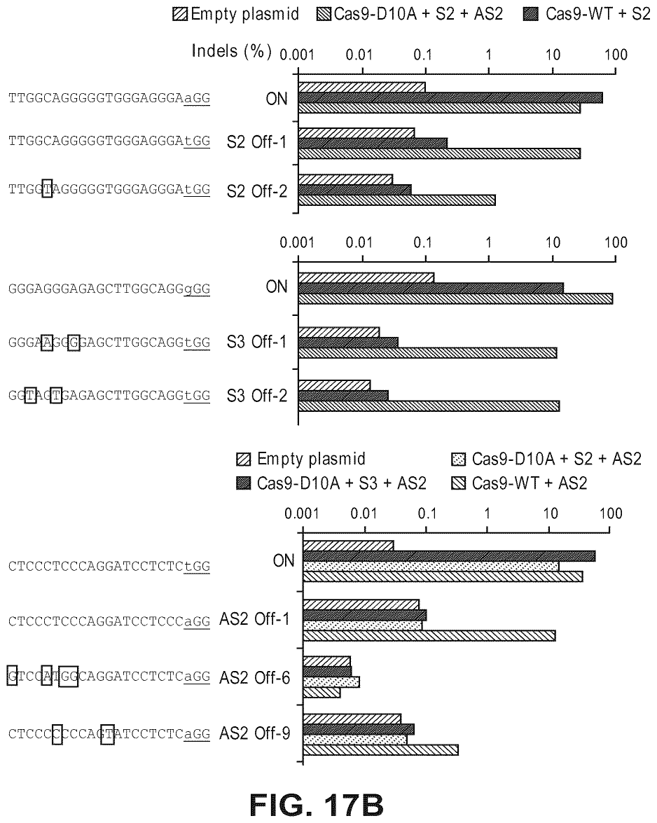

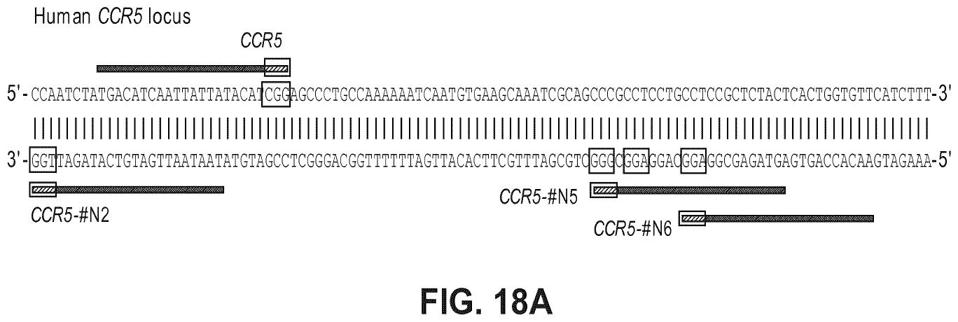

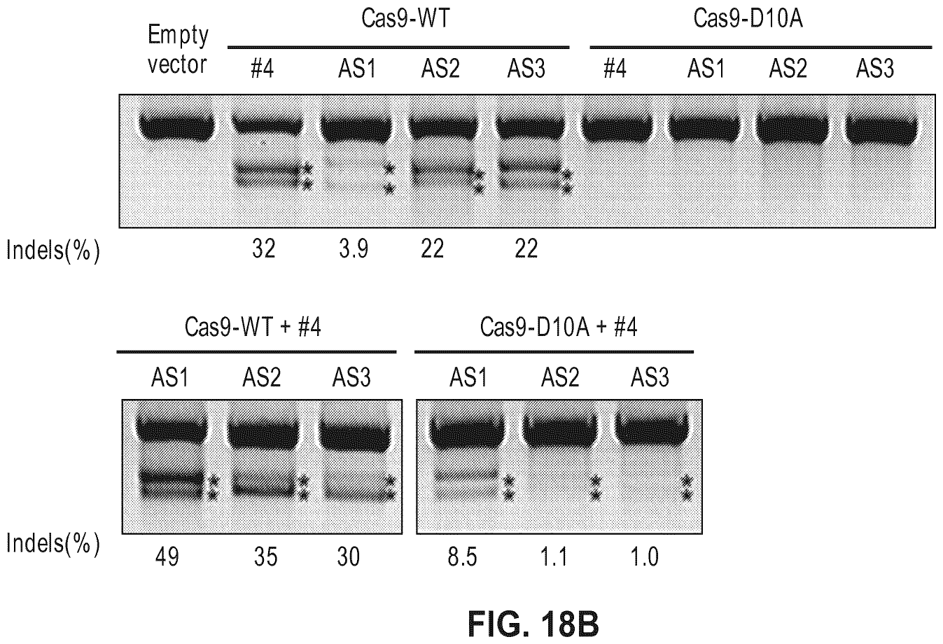

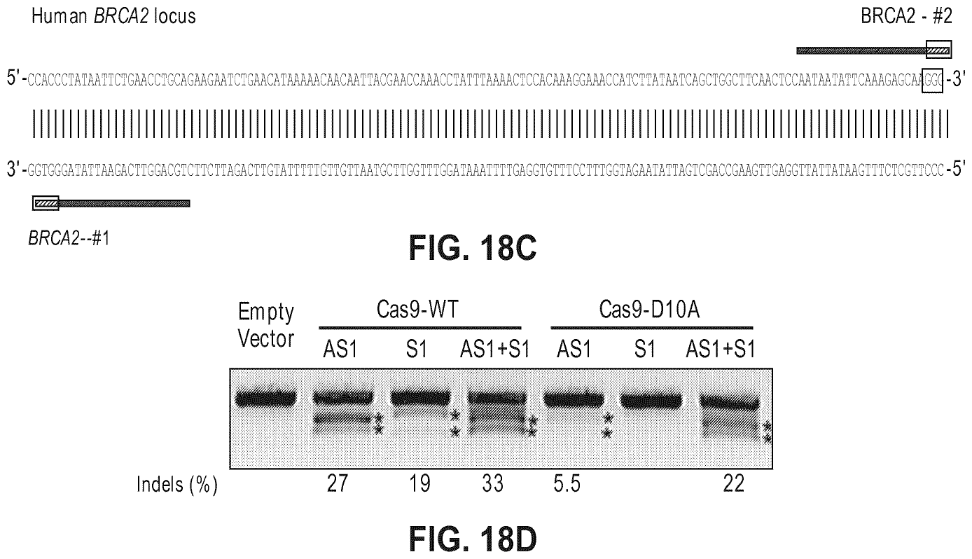

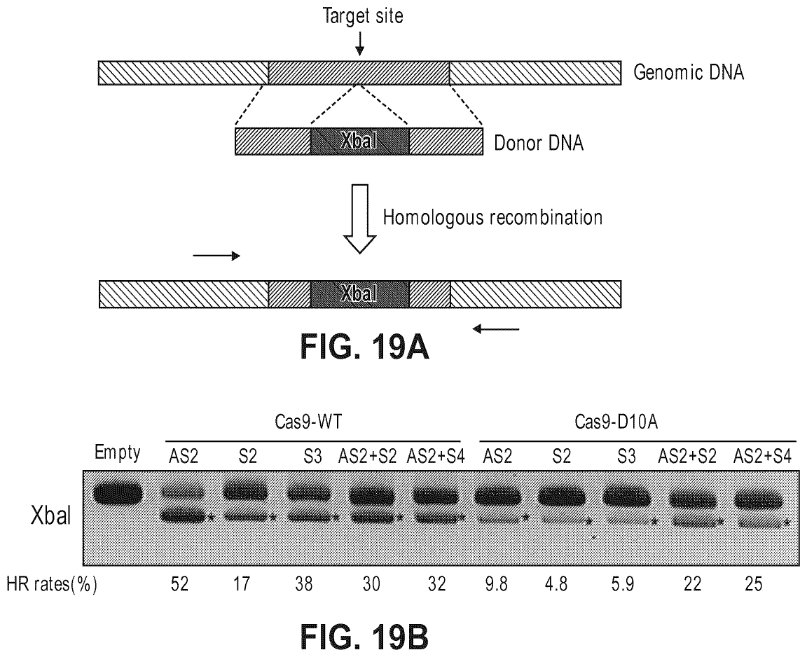

We first tested several Cas9 nucleases and nickases designed to target sites in the AAVS1 locus (FIG. 16B) in vitro via fluorescent capillary electrophoresis. Unlike Cas9 nucleases that cleaved both strands of DNA substrates, Cas9 nickases composed of guide RNA and a mutant form of Cas9 in which a catalytic aspartate residue is changed to an alanine (D10A Cas9) cleaved only one strand, producing site-specific nicks (FIG. 16C,D). Interestingly, however, some nickases (AS1, AS2, AS3, and S6 in FIG. 17A) induced indels at target sites in human cells, suggesting that nicks can be converted to DSBs, albeit inefficiently, in vivo. Paired Cas9 nickases producing two adjacent nicks on opposite DNA strands yielded indels at frequencies that ranged from 14% to 91%, comparable to the effects of paired nucleases (FIG. 17A). The repair of two nicks that would produce 5′ overhangs led to the formation of indels much more frequently than those producing 3′ overhangs at three genomic loci (FIG. 17A and FIG. 18). In addition, paired nickases enabled targeted genome editing via homology-directed repair more efficiently than did single nickases (FIG. 19).

We next measured mutation frequencies of paired nickases and nucleases at off-target sites using deep sequencing. Cas9 nucleases complexed with three sgRNAs induced off-target mutations at six sites that differ by one or two nucleotides from their corresponding on-target sites with frequencies that ranged from 0.5% to 10% (FIG. 17B). In contrast, paired Cas9 nickases did not produce indels above the detection limit of 0.1% at any of the six off-target sites. The S2 Off-1 site that differs by a single nucleotide at the first position in the PAM (i.e., N in NGG) from its on-target site can be considered as another on-target site. As expected, the Cas9 nuclease complexed with the S2 sgRNA was equally efficient at this site and the on-target site. In sharp contrast, D10A Cas9 complexed with the S2 and AS2 sgRNAs discriminated this site from the on-target site by a factor of 270 fold. This paired nickase also discriminated the AS2 off-target sites (Off-1 and Off-9 in FIG. 17B) from the on-target site by factors of 160 fold and 990 fold, respectively.

### Example 8: Chromosomal DNA Splicing Induced by Paired Cas9 Nickases

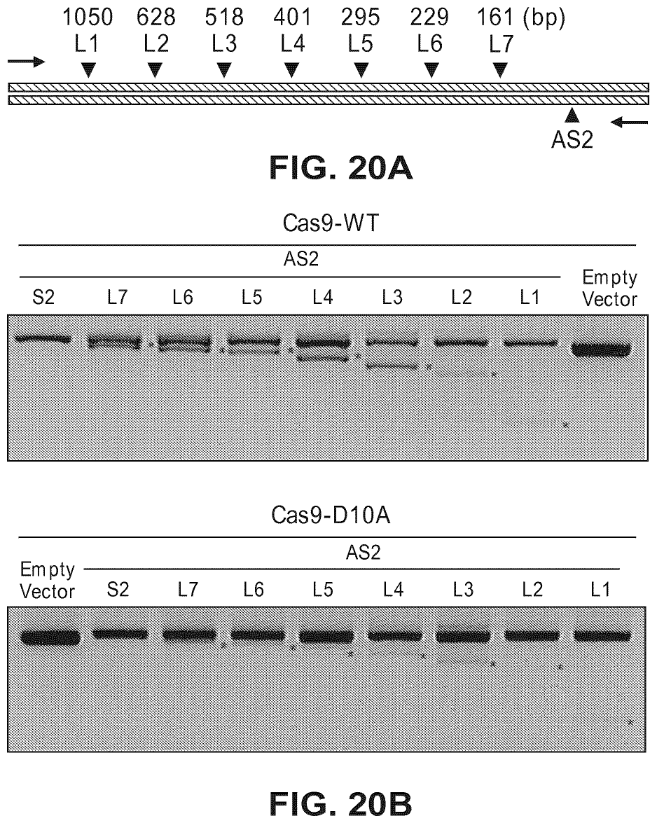

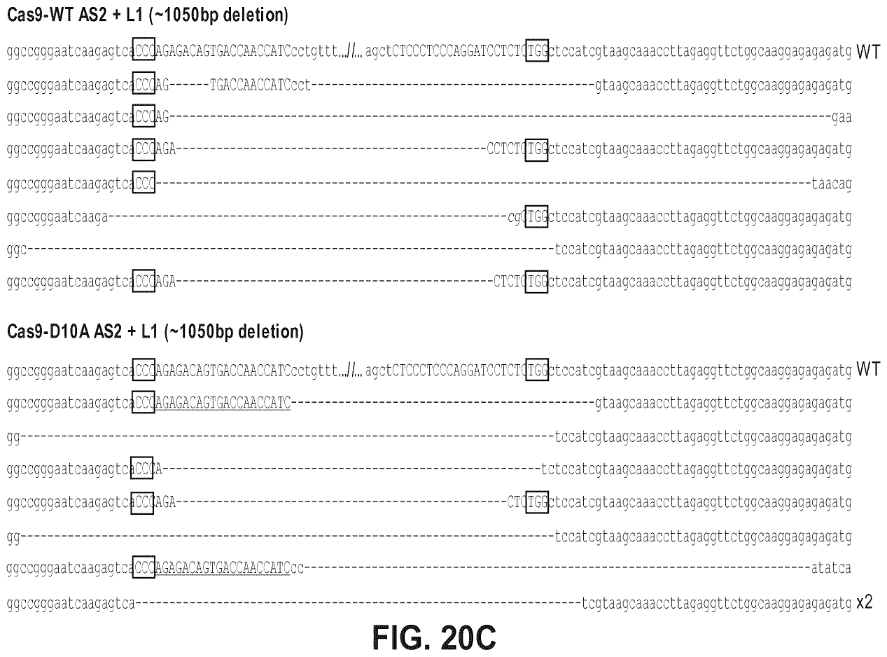

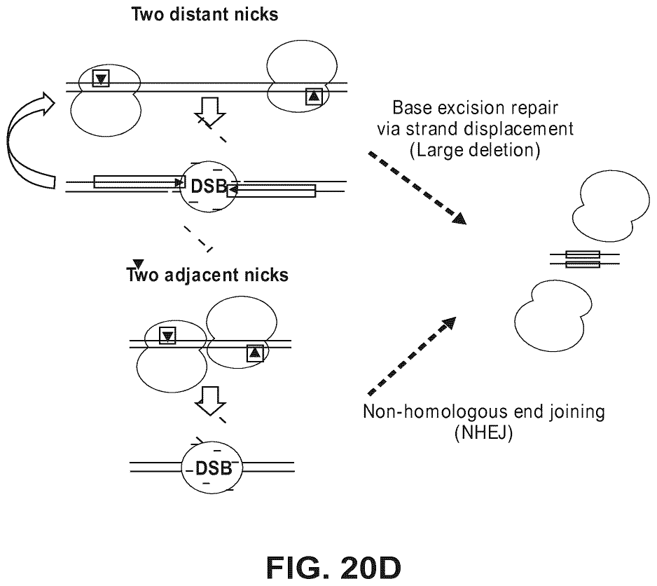

Two concurrent DSBs produced by engineered nucleases such as ZFNs and TALENs can promote large deletions of the intervening chromosomal segments has been reported. We tested whether two SSBs induced by paired Cas9 nickases can also produce deletions in human cells. We used PCR to detect deletion events and found that seven paired nickases induced deletions of up to 1.1-kbp chromosomal segments as efficiently as paired Cas9 nucleases did (FIG. 20A,B). DNA sequences of the PCR products confirmed the deletion events (FIG. 20C). Interestingly, the sgRNA-matching sequence remained intact in two out of seven deletion-specific PCR amplicons (underlined in FIG. 20C). In contrast, Cas9 nuclease pairs did not produce sequences that contained intact target sites. This finding suggests that two distant nicks were not converted to two separate DSBs to promote deletions of the intervening chromosomal segment. In addition, it is unlikely that two nicks separated by more than a 100 bp can produce a composite DSB with large overhangs under physiological conditions because the melting temperature is very high.

We propose that two distant nicks are repaired by strand displacement in a head-to-head direction, resulting in the formation of a DSB in the middle, whose repair via NHEJ causes small deletions (FIG. 20D). Because the two target sites remain intact during this process, nickases can induce SSBs again, triggering the cycle repeatedly until the target sites are deleted. This mechanism explains why two offset nicks producing 5′ overhangs but not those producing 3′ overhangs induced indels efficiently at three loci.

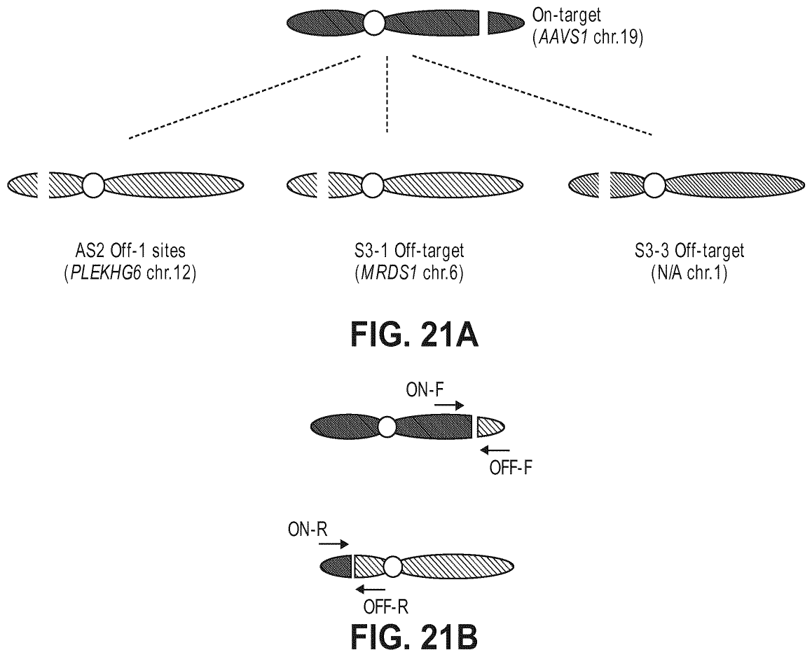

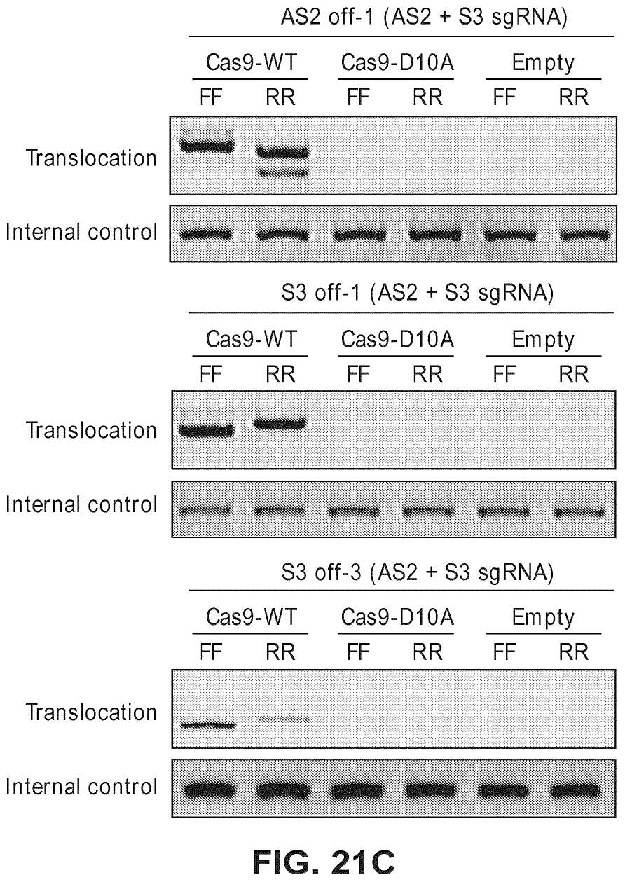

We then investigated whether Cas9 nucleases and nickases can induce unwanted chromosomal translocations that result from NHEJ repair of on-target and off-target DNA cleavages (FIG. 21A). We were able to detect translocations induced by Cas9 nucleases using PCR (FIG. 21B,C). No such PCR products were amplified using genomic DNA isolated from cells transfected with the plasmids encoding the AS2+S3 Cas9 nickase pair. This result is in line with the fact that both AS2 and S3 nickases, unlike their corresponding nucleases, did not produce indels at off-target sites (FIG. 17B).

These results suggest that paired Cas9 nickases allow targeted mutagenesis and large deletions of up to 1-kbp chromosomal segments in human cells. Importantly, paired nickases did not induce indels at off-target sites at which their corresponding nucleases induce mutations. Furthermore, unlike nucleases, paired nickases did not promote unwanted translocations associated with off-target DNA cleavages. In principle, paired nickases double the specificity of Cas9-mediated mutagenesis and will broaden the utility of RNA-guided enzymes in applications that require precise genome editing such as gene and cell therapy. One caveat to this approach is that two highly active sgRNAs are needed to make an efficient nickase pair, limiting targetable sites. As shown in this and other studies, not all sgRNAs are equally active. When single clones rather than populations of cells are used for further studies or applications, the choice of guide RNAs that represent unique sequences in the genome and the use of optimized guide RNAs would suffice to avoid off-target mutations associated with Cas9 nucleases. We propose that both Cas9 nucleases and paired nickases are powerful options that will facilitate precision genome editing in cells and organisms.

### Example 9: Genotyping with CRISPR/Cas-Derived RNA-Guided Endonucleases

Next, we reasoned that RGENs can be used in Restriction fragment length polymorphism (RFLP) analysis, replacing conventional restriction enzymes. Engineered nucleases including RGENs induce indels at target sites, when the DSBs caused by the nucleases are repaired by the error-prone non-homologous end-joining (NHEJ) system. RGENs that are designed to recognize the target sequences cannot cleave mutant sequences with indels but will cleave wildtype target sequences efficiently.

### 9-1. RGEN Components

crRNA and tracrRNA were prepared by in vitro transcription using MEGAshortcript T7 kit (Ambion) according to the manufacturer's instruction. Transcribed RNAs were resolved on a 8% denaturing urea-PAGE gel. The gel slice containing RNA was cut out and transferred to elution buffer. RNA was recovered in nuclease-free water followed by phenol:chloroform extraction, chloroform extraction, and ethanol precipitation. Purified RNA was quantified by spectrometry. Templates for crRNA were prepared by annealing an oligonucleotide whose sequence is shown as 5′-GAAATTAATACGACTCACTATAGGX₂₀GTTTTAGAGCTATGCTGTTTTG-3′ (SEQ ID NO: 76), in which X₂₀ is the target sequence, and its complementary oligonucleotide. The template for tracrRNA was synthesized by extension of forward and reverse oligonucleotides (5′-GAAATTAATACGACTCACTATAGGAACCATTCAAAACAGCATAGCAAGTTAAAATAAGGCTA GTCCG-3′ (SEQ ID NO: 77) and 5′-AAAAAAAGCACCGACTCGGTGCCACTTTTTCAAGTTGATAACGGACTAGCCTTATTTTAACT TGCTATG-3′ (SEQ ID NO: 78)) using Phusion polymerase (New England Biolabs).

### 9-2. Recombinant Cas9 Protein Purification

The Cas9 DNA construct used in our previous Example, which encodes Cas9 fused to the His6-tag at the C terminus, was inserted in the pET-28a expression vector. The recombinant Cas9 protein was expressed in E. coli strain BL21(DE3) cultured in LB medium at 25° C. for 4 hours after induction with 1 mM IPTG. Cells were harvested and resuspended in buffer containing 20 mM Tris PH 8.0, 500 mM NaCl, 5 mM imidazole, and 1 mM PMSF. Cells were frozen in liquid nitrogen, thawed at 4° C., and sonicated. After centrifugation, the Cas9 protein in the lysate was bound to Ni-NTA agarose resin (Qiagen), washed with buffer containing 20 mM Tris pH 8.0, 500 mM NaCl, and 20 mM imidazole, and eluted with buffer containing 20 mM Tris pH 8.0, 500 mM NaCl, and 250 mM imidazole. Purified Cas9 protein was dialyzed against 20 mM HEPES (pH 7.5), 150 mM KCl, 1 mM DTT, and 10% glycerol and analyzed by SDS-PAGE.

### 9-3. T7 Endonuclease I Assay

The T7E1 assay was performed as following. In brief, PCR products amplified using genomic DNA were denatured at 95° C., reannealed at 16° C., and incubated with 5 units of T7 Endonuclease I (New England BioLabs) for 20 min at 37° C. The reaction products were resolved using 2 to 2.5% agarose gel electrophoresis.

### 9-4. RGEN-RFLP Assay

PCR products (100-150 ng) were incubated for 60 min at 37° C. with optimized concentrations (Table 10) of Cas9 protein, tracrRNA, crRNA in 10 μl NEB buffer 3 (1×). After the cleavage reaction, RNase A (4 μg) was added, and the reaction mixture was incubated for 30 min at 37° C. to remove RNA. Reactions were stopped with 6× stop solution buffer containing 30% glycerol, 1.2% SDS, and 100 mM EDTA. Products were resolved with 1-2.5% agarose gel electrophoresis and visualized with EtBr staining.

TABLE 10
Concentration of RGEN components in RFLP assays

| | Cas9 | crRNA | tracrRNA |
|---|---|---|---|
| Target Name | (ng/μl) | (ng/μl) | (ng/μl) |
| C4BPB | 100 | 25 | 60 |
| PIBF-NGG-RGEN | 100 | 25 | 60 |
| HLA-B | 1.2 | 0.3 | 0.7 |
| CCR5-ZFN | 100 | 25 | 60 |
| CTNNB1 Wild type specific | 30 | 10 | 20 |
| CTNNB1 mutant specific | 30 | 10 | 20 |
| CCR5 WT-specific | 100 | 25 | 60 |
| CCR5 Δ32-specific | 10 | 2.5 | 6 |
| KRAS WT specific(wt) | 30 | 10 | 20 |
| KRAS mutant specific(m8) | 30 | 10 | 20 |
| KRAS WT specific (m6) | 30 | 10 | 20 |
| KRAS mutant specific (m6, 8) | 30 | 10 | 20 |
| PIK3CA WT specific (wt) | 100 | 25 | 60 |
| PIK3CA mutant specific(m4) | 30 | 10 | 20 |
| PIK3CA WT specific (m7) | 100 | 25 | 60 |
| PIK3CA mutant specific(m4, 7) | 30 | 10 | 20 |
| BRAF WT-specific | 30 | 10 | 20 |
| BRAF mutant-specific | 100 | 25 | 60 |
| NRAS WT-specific | 100 | 25 | 60 |
| NRAS mutant-specific | 30 | 10 | 20 |
| IDH WT-specific | 30 | 10 | 20 |
| IDH mutant-specific | 30 | 10 | 20 |
| PIBF-NAG-RGEN | 30 | 10 | 60 |

TABLE 11
Primers

| Gene(site) | Direction | Sequence(5′ to 3′) | SEQ ID NO |
|---|---|---|---|
| CCR5(RGEN) | F1 | CTCCATGGTGCTATAGAGCA | 79 |
| | F2 | GAGCCAAGCTCTCCATCTAGT | 80 |
| | R | GCCCTGTCAAGAGTTGACAC | 81 |
| CCR5(ZFN) | F | GCACAGGGTGGAACAAGATGGA | 82 |
| | R | GCCAGGTACCTATCGATTGTCAGG | 83 |
| CCR5(del32) | F | GAGCCAAGCTCTCCATCTAGT | 84 |
| | R | ACTCTGACTG GGTCACCAGC | 85 |
| C4BPB | F1 | TATTTGGCTGGTTGAAAGGG | 86 |
| | R1 | AAAGTCATGAAATAAACACACCCA | 87 |
| | F2 | CTGCATTGATATGGTAGTACCATG | 88 |
| | R2 | GCTGTTCATTGCAATGGAATG | 89 |
| CTNNB1 | F | ATGGAGTTGGACATGGCCATGG | 90 |
| | R | ACTCACTATCCACAGTTCAGCATTTACC | 91 |
| KRAS | F | TGGAGATAGCTGTCAGCAACTTT | 92 |
| | R | CAACAA AGCAAAGGTAAAGTTGGTAATAG | 93 |
| PIK3CA | F | GGTTTCAGGAGATGTGTTACAAGGC | 94 |
| | R | GATTGTGCAATTCCTATGCAATCGGTC | 95 |
| NRAS | F | CACTGGGTACTTAATCTGTAGCCTC | 96 |
| | R | GGTTCCAAGTCATTCCCAGTAGC | 97 |
| IDH1 | F | CATCACTGCAGTTGTAGGTTATAACTATCC | 98 |
| | R | TTGAAAACCACAGATCTGGTTGAACC | 99 |
| BRAF | F | GGAGTGCCAAGAGAATATCTGG | 100 |
| | R | CTGAAACTGGTTTCAAAATATTCGTTTTAAGG | 101 |
| PIBF | F | GCTCTGTATGCCCTGTAGTAGG | 102 |
| | R | TTTGCATCTGACCTTACCTTTG | 103 |

### 9-5. Plasmid Cleavage Assay

Restriction enzyme-treated linearized plasmid (100 ng) was incubated for 60 min at 37° C. with Cas9 protein (0.1 μg), tracrRNA (60 ng), and crRNA (25 ng) in 10 μl NEB 3 buffer (1×). Reactions were stopped with 6× stop solution containing 30% glycerol, 1.2% SDS, and 100 mM EDTA. Products were resolved with 1% agarose gel electrophoresis and visualized with EtBr staining.

### 9-6. Strategy of RFLP

New RGENs with desired DNA specificities can be readily created by replacing crRNA; no de novo purification of custom proteins is required once recombinant Cas9 protein is available. Engineered nucleases, including RGENs, induce small insertions or deletions (indels) at target sites when the DSBs caused by the nucleases are repaired by error-prone non-homologous end-joining (NHEJ). RGENs that are designed to recognize the target sequences cleave wild-type sequences efficiently but cannot cleave mutant sequences with indels (FIG. 22).

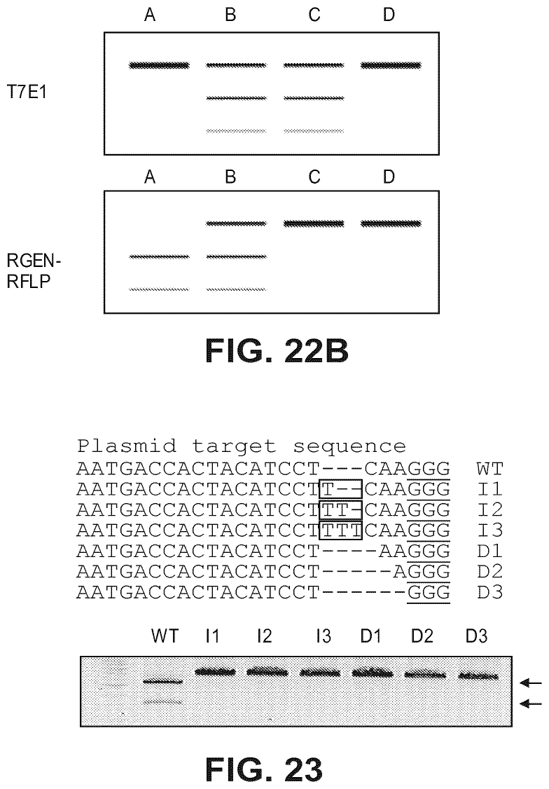

We first tested whether RGENs can differentially cleave plasmids that contain wild-type or modified C4BPB target sequences that harbor 1- to 3-base indels at the cleavage site. None of the six plasmids with these indels were cleaved by a C4BPB-specific RGEN5 composed of target-specific crRNA, tracrRNA, and recombinant Cas9 protein (FIG. 23). In contrast, the plasmid with the intact target sequence was cleaved efficiently by this RGEN.

### 9-7. Detection of Mutations Induced by the Same RGENs Using RGEN-Mediated RFLP

Next, to test the feasibility of RGEN-mediated RFLP for detection of mutations induced by the same RGENs, we utilized gene-modified K562 human cancer cell clones established using an RGEN targeting C4BPB gene (Table 12).

TABLE 12
Target sequence of RGENs used in this study

| Gene | Target sequence | SEQ ID NO |
|---|---|---|
| human C4BPB | AATGACCACTACATCCTCAAGGG | 104 |
| mouse Pibf1 | AGATGATGTCTCATCATCAGAGG | 105 |

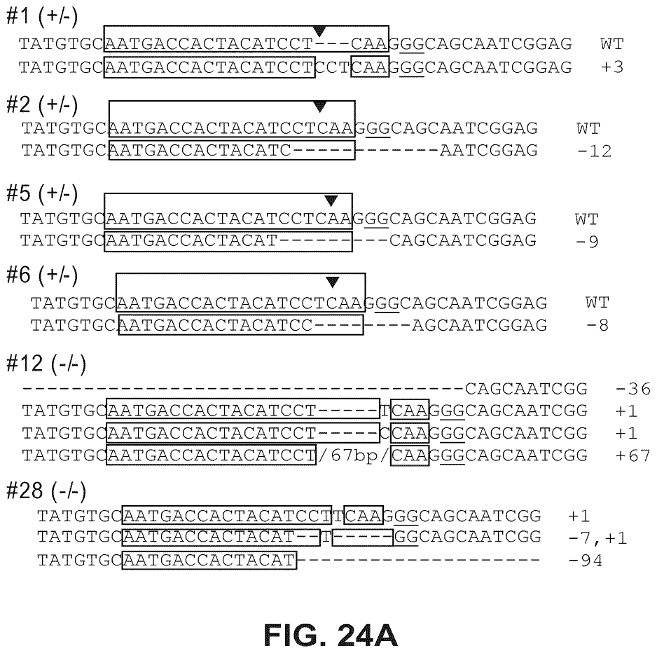

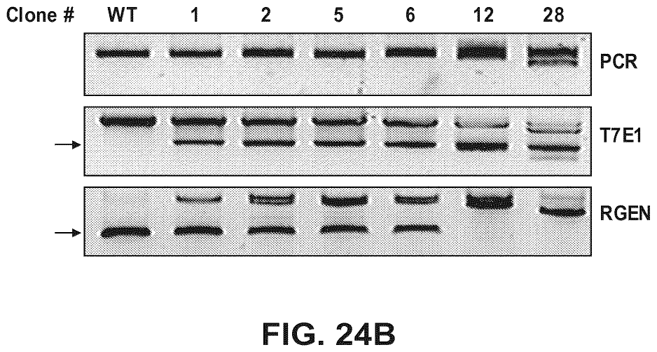

C4BPB mutant clones used in this study have various mutations ranging from 94 bp deletion to 67 bp insertion (FIG. 24A). Importantly, all mutations occurred in mutant clones resulted in the loss of RGEN target site. Among 6 C4BPB clones analyzed, 4 clones have both wildtype and mutant alleles (+/−) and 2 clones have only mutant alleles (−/−).

The PCR products spanning the RGEN target site amplified from wildtype K562 genomic DNA were digested completely by the RGEN composed of target-specific crRNA, tracrRNA, and recombinant Cas9 protein expressed in and purified from E. coli (FIG. 24B/Lane 1). When the C4BPB mutant clones were subjected to RFLP analysis using the RGEN, PCR amplicons of +/−clones that contained both wildtype and mutant alleles were partially digested, and those of −/− cloned that did not contain the wildtype allele were not digested at all, yielding no cleavage products corresponding to the wildtype sequence (FIG. 24B). Even a single-base insertion at the target site blocked the digestion (#12 and #28 clones) of amplified mutant alleles by the C4BPB RGEN, showing the high specificity of RGEN-mediated RFLP. We subjected the PCR amplicons to the mismatch-sensitive T7E1 assay in parallel (FIG. 24B). Notably, the T7E1 assay was not able to distinguish −/− clones from +/−clones. To make it matters worse, the T7E1 assay cannot distinguish homozygous mutant clones that contain the same mutant sequence from wildtype clones, because annealing of the same mutant sequence will form a homoduplex. Thus, RGEN-mediated RFLP has a critical advantage over the conventional mismatch-sensitive nuclease assay in the analysis of mutant clones induced by engineered nucleases including ZFNs, TALENs and RGENs.

### 9-8. Quantitative Assay for RGEN-RFLP Analysis

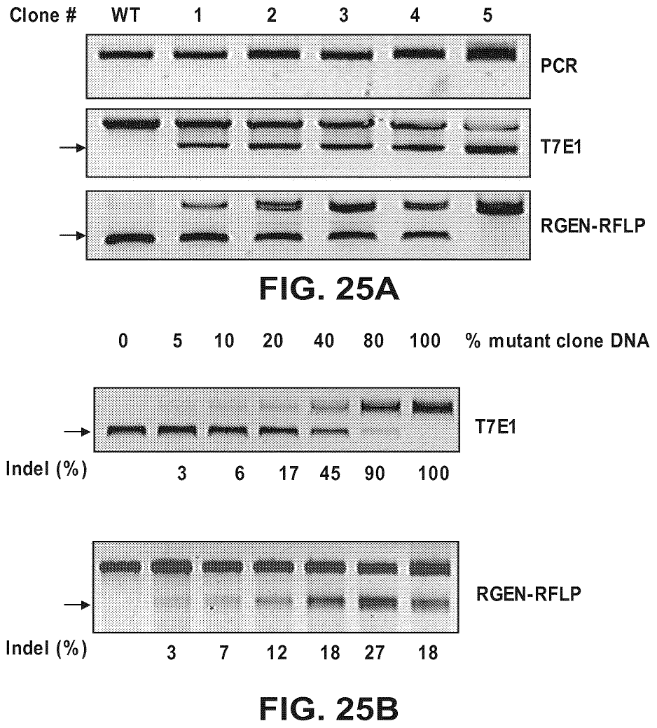

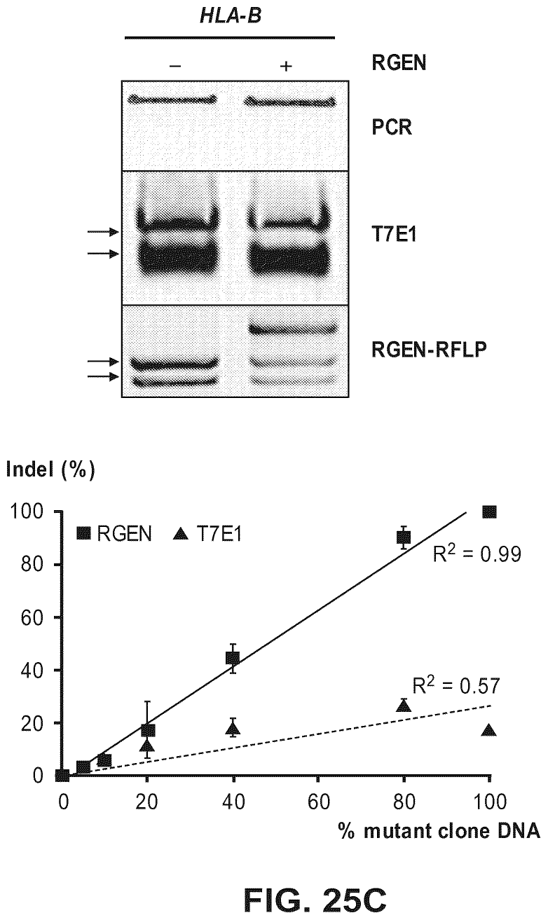

We also investigated whether RGEN-RFLP analysis is a quantitative method. Genomic DNA samples isolated from the C4BPB null clone and the wild-type cells were mixed at various ratios and used for PCR amplifications. The PCR products were subjected to RGEN genotyping and the T7E1 assay in parallel (FIG. 25b). As expected, DNA cleavage by the RGEN was proportional to the wild type to mutant ratio. In contrast, results of the T7E1 assay correlated poorly with mutation frequencies inferred from the ratios and were inaccurate, especially at high mutant %, a situation in which complementary mutant sequences can hybridize with each other to form homoduplexes.

### 9-9. Analysis of Mutant Mouse Founders Using a RGEN-Mediated RFLP Genotyping

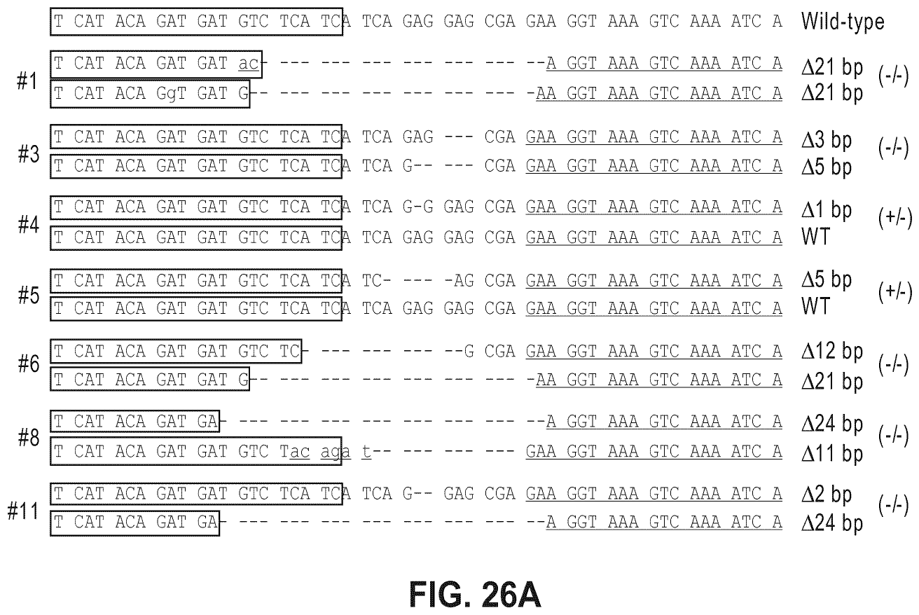

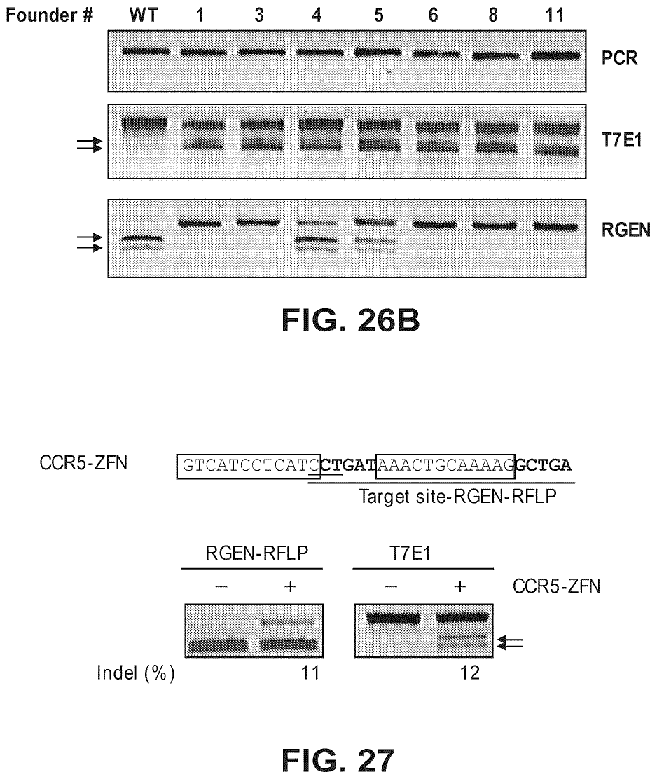

We also applied RGEN-mediated RFLP genotyping (RGEN genotyping in short) to the analysis of mutant mouse founders that had been established by injection of TALENs into mouse one-cell embryos (FIG. 26A). We designed and used an RGEN that recognized the TALEN target site in the Pibf1 gene (Table 10). Genomic DNA was isolated from a wildtype mouse and mutant mice and subjected to RGEN genotyping after PCR amplification. RGEN genotyping successfully detected various mutations, which ranged from one to 27-bp deletions (FIG. 26B). Unlike the T7E1 assay, RGEN genotyping enabled differential detection of +/− and −/− founder.

### 9-10. Detection of Mutations Induced in Human Cells by a CCR5-Specific ZFN Using RGENs

In addition, we used RGENs to detect mutations induced in human cells by a CCR5-specific ZFN, representing yet another class of engineered nucleases (FIG. 27). These results show that RGENs can detect mutations induced by nucleases other than RGENs themselves. In fact, we expect that RGENs can be designed to detect mutations induced by most, if not all, engineered nucleases. The only limitation in the design of an RGEN genotyping assay is the requirement for the GG or AG (CC or CT on the complementary strand) dinucleotide in the PAM sequence recognized by the Cas9 protein, which occurs once per 4 bp on average. Indels induced anywhere within the seed region of several bases in crRNA and the PAM nucleotides are expected to disrupt RGEN-catalyzed DNA cleavage. Indeed, we identified at least one RGEN site in most (98%) of the ZFN and TALEN sites.

### 9-11. Detection of Polymorphisms or Variations Using RGEN

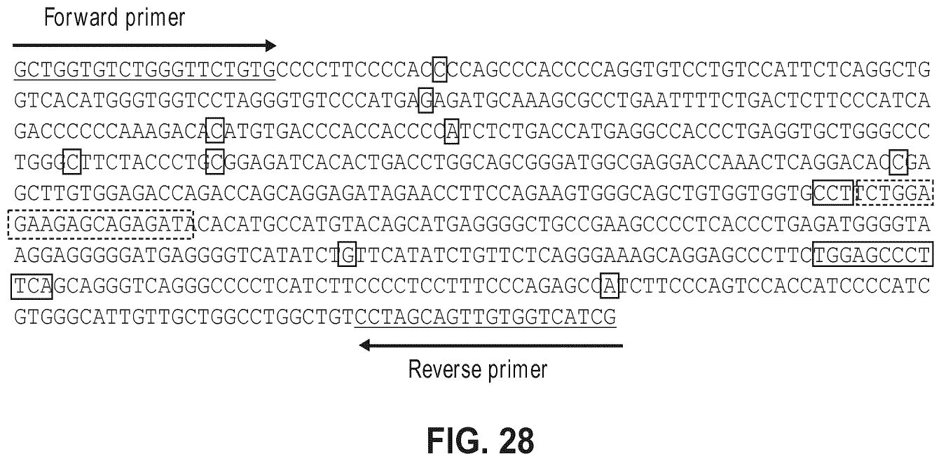

Next, we designed and tested a new RGEN that targets a highly polymorphic locus, HLA-B, that encodes Human Leukocyte Antigen B (a.k.a. MHC class I protein) (FIG. 28). HeLa cells were transfected with RGEN plasmids, and the genomic DNA was subjected to T7E1 and RGEN-RFLP analyses in parallel. T7E1 produced false positive bands that resulted from sequence polymorphisms near the target site (FIG. 25c). As expected, however, the same RGEN used for gene disruption cleaved PCR products from wild-type cells completely but those from RGEN-transfected cells partially, indicating the presence of RGEN-induced indels at the target site. This result shows that RGEN-RFLP analysis has a clear advantage over the T7E1 assay, especially when it is not known whether target genes have polymorphisms or variations in cells of interest.

### 9-12. Detection of Recurrent Mutations Found in Cancer and Naturally-Occurring Polymorphisms Through RGEN-RFLP Analysis

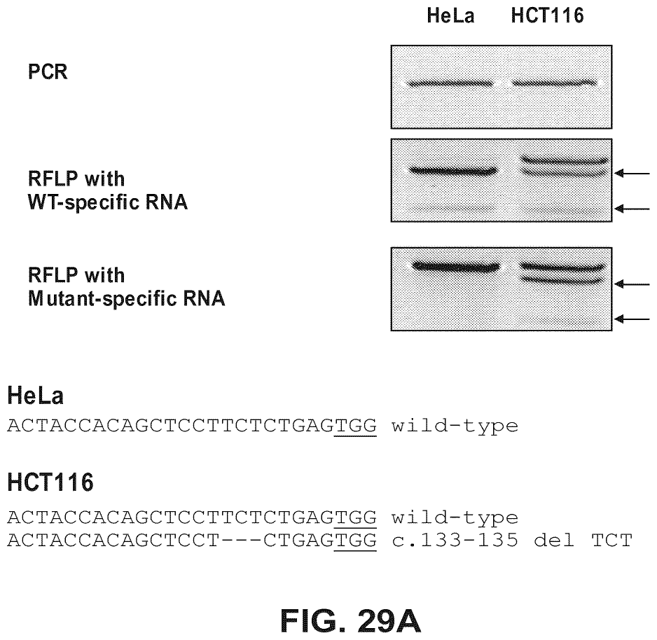

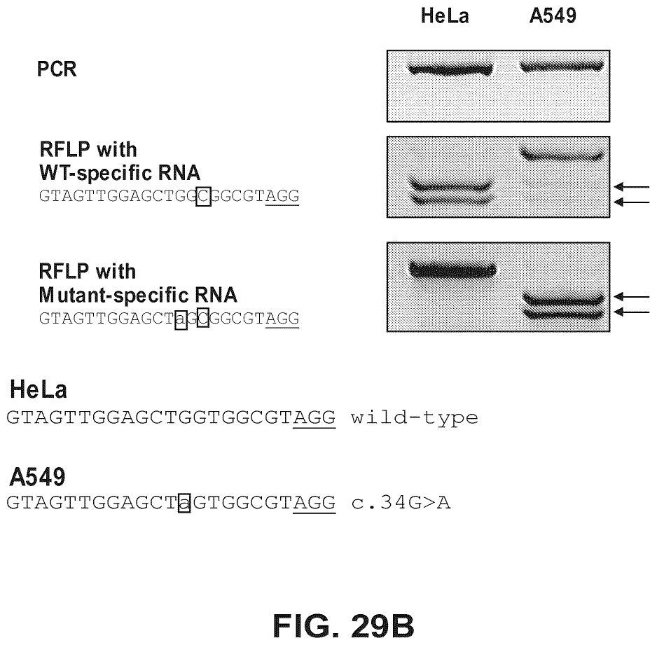

RGEN-RFLP analysis has applications beyond genotyping of engineered nuclease-induced mutations. We sought to use RGEN genotyping to detect recurrent mutations found in cancer and naturally-occurring polymorphisms. We chose the human colorectal cancer cell line, HCT116, which carries a gain-of-function 3-bp deletion in the oncogenic CTNNB1 gene encoding beta-catenin. PCR products amplified from HCT116 genomic DNA were cleaved partially by both wild-type-specific and mutant-specific RGENs, in line with the heterozygous genotype in HCT116 cells (FIG. 29a). In sharp contrast, PCR products amplified from DNA from HeLa cells harboring only wild-type alleles were digested completely by the wild-type-specific RGEN and were not cleaved at all by the mutation-specific RGEN.

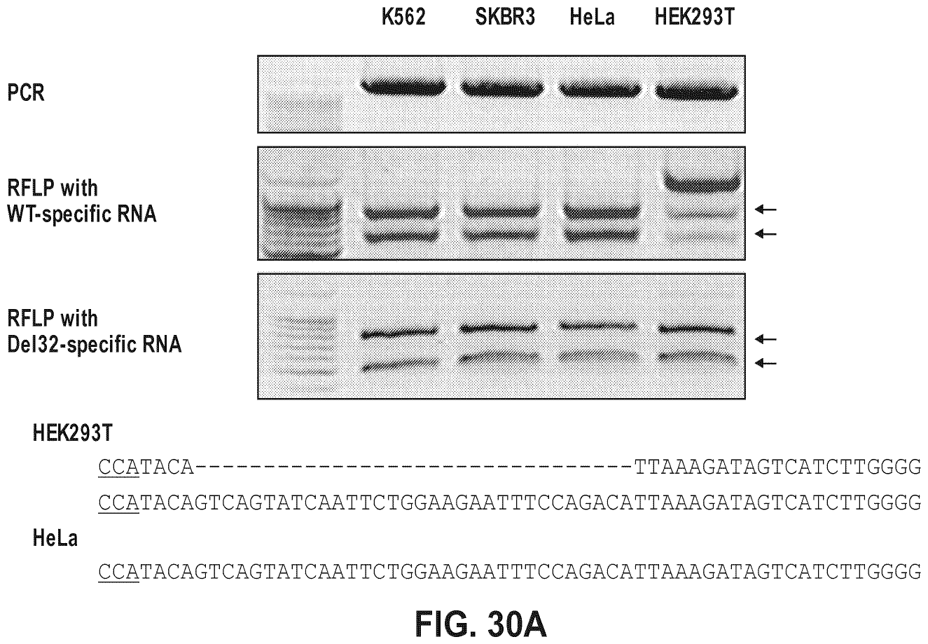

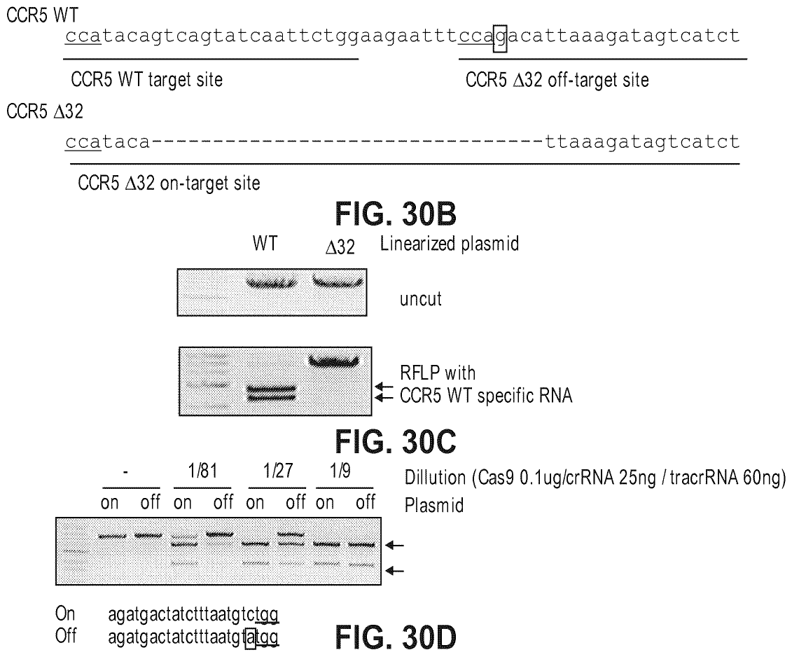

We also noted that HEK293 cells harbor the 32-bp deletion (del32) in the CCR5 gene, which encodes an essential co-receptor of HIV infection: Homozygous del32 CCR5 carriers are immune to HIV infection. We designed one RGEN specific to the del32 allele and the other to the wild-type allele. As expected, the wild-type-specific RGEN cleaved the PCR products obtained from K562, SKBR3, or HeLa cells (used as wild-type controls) completely but those from HEK293 cells partially (FIG. 30a), confirming the presence of the uncleavable del32 allele in HEK293 cells. Unexpectedly, however, the del32-specific RGEN cleaved the PCR products from wild-type cells as efficiently as those from HEK293 cells. Interestingly, this RGEN had an off-target site with a single-base mismatch immediately downstream of the on-target site (FIG. 30). These results suggest that RGENs can be used to detect naturally-occurring indels but cannot distinguish sequences with single nucleotide polymorphisms or point mutations due to their off-target effects.

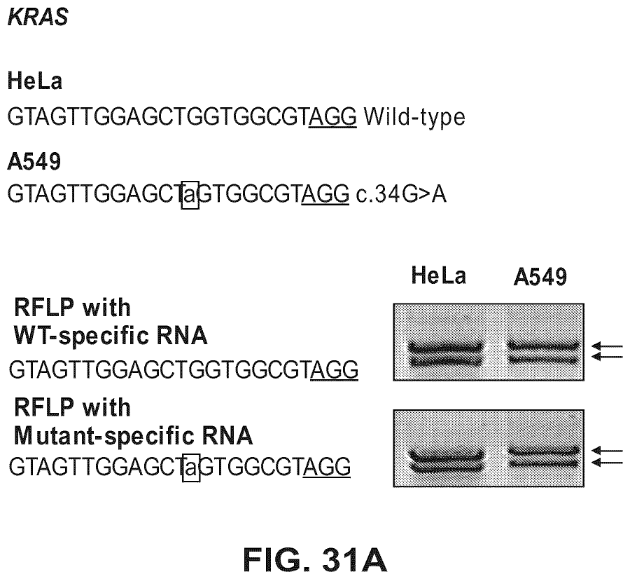

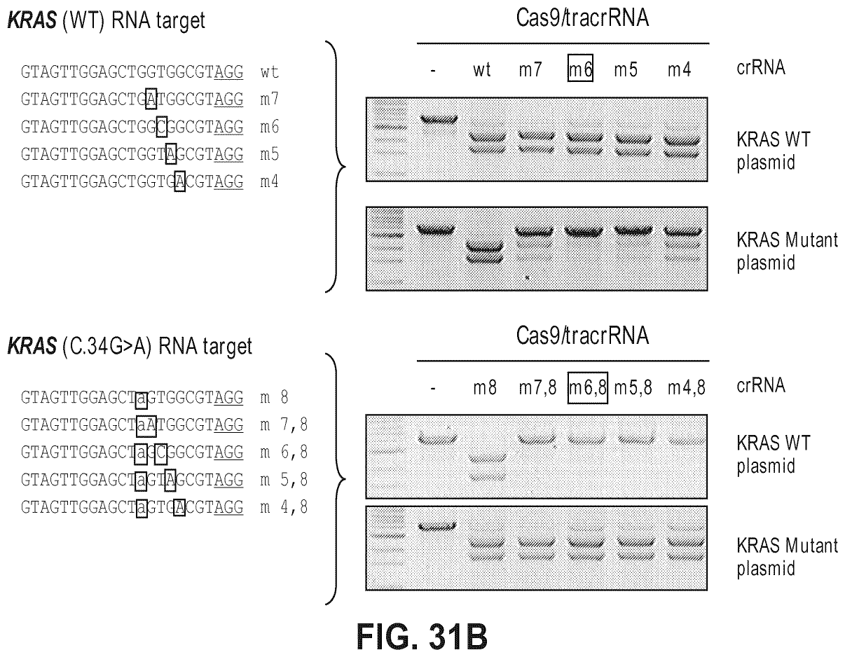

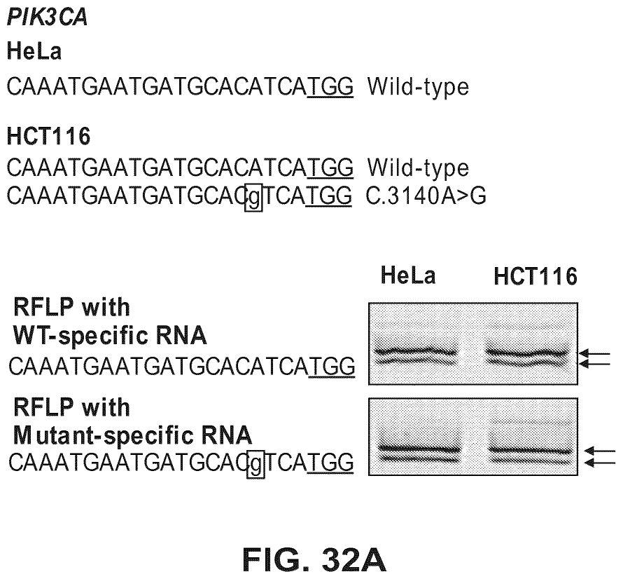

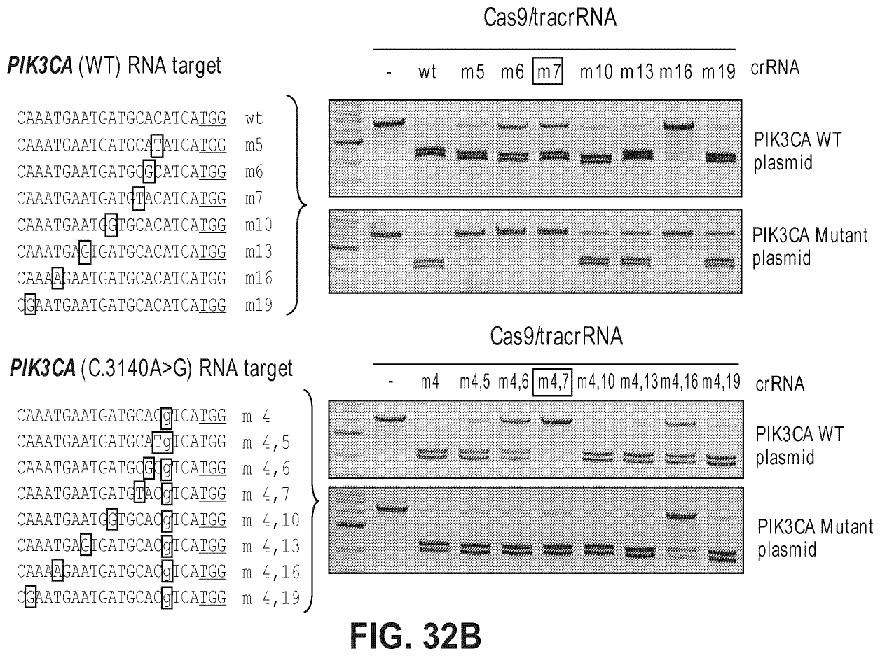

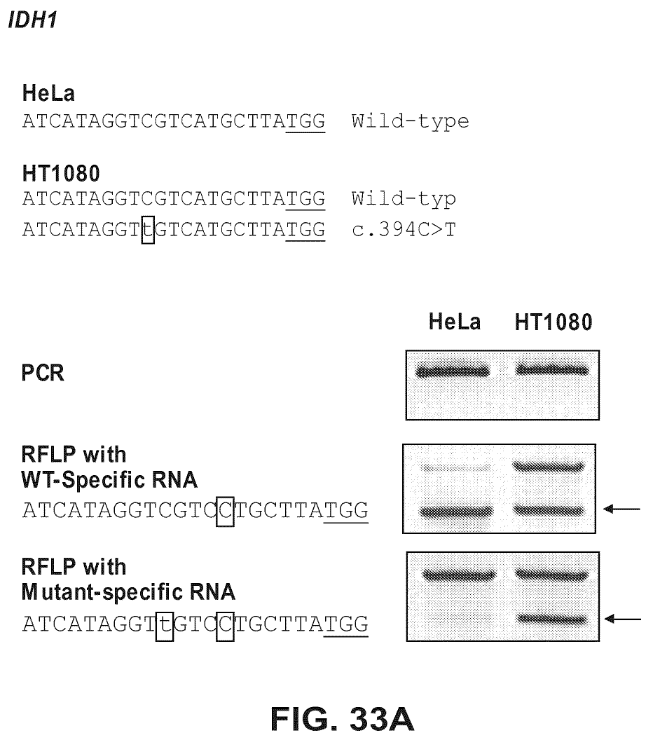

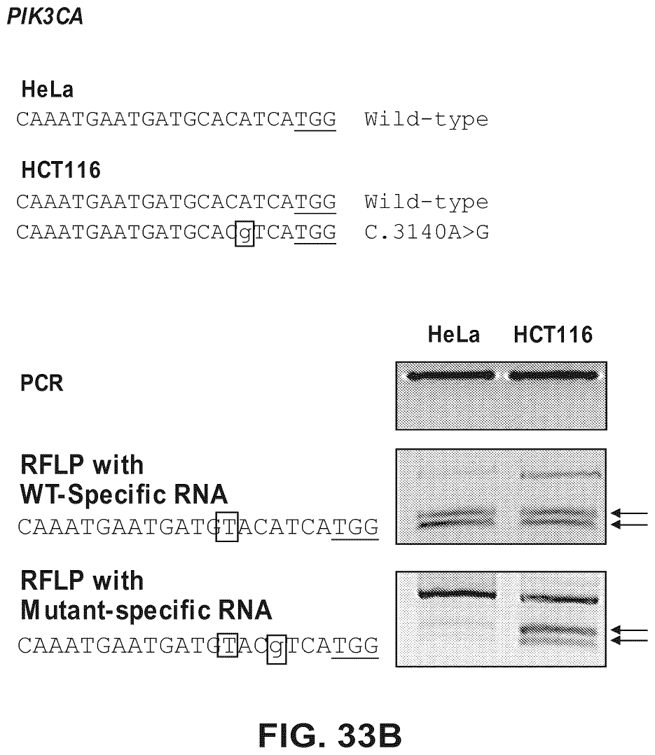

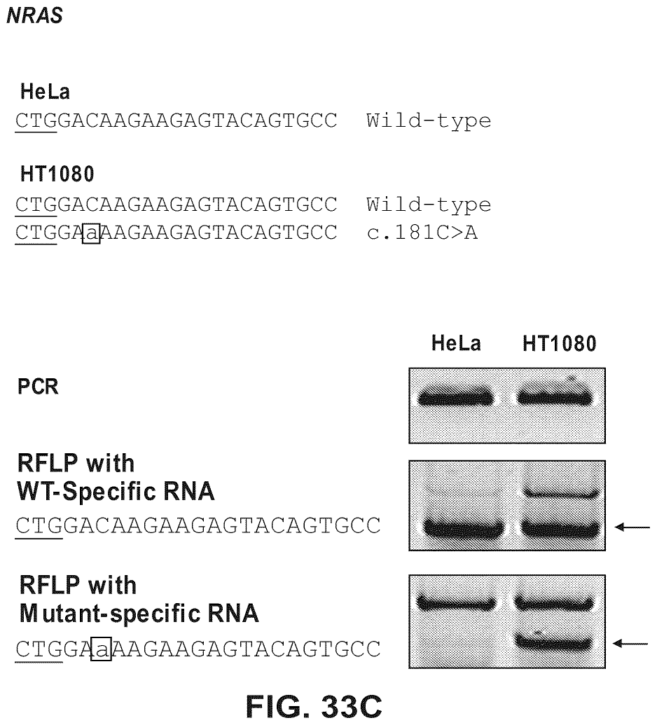

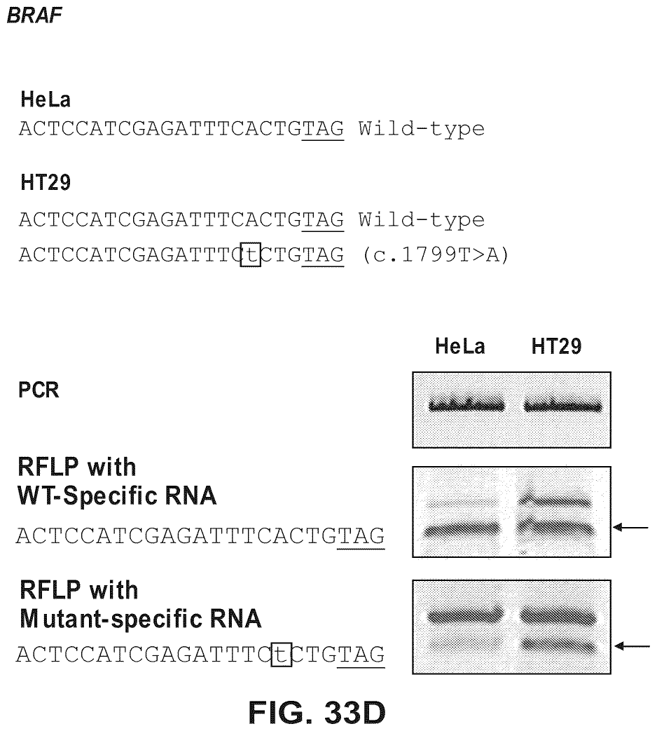

To genotype oncogenic single-nucleotide variations using RGENs, we attenuated RGEN activity by employing a single-base mismatched guide RNA instead of a perfectly-matched RNA. RGENs that contained the perfectly-matched guide RNA specific to the wild-type sequence or mutant sequence cleaved both sequences (FIGS. 31a and 32a). In contrast, RGENs that contained a single-base mismatched guide RNA distinguished the two sequences, enabling genotyping of three recurrent oncogenic point mutations in the KRAS, PIK3CA, and IDH1 genes in human cancer cell lines (FIG. 29b and FIG. 33a, b). In addition, we were able to detect point mutations in the BRAF and NRAS genes using RGENs that recognize the NAG PAM sequence (FIG. 33c, d). We believe that we can use RGEN-RFLP to genotype almost any, if not all, mutations or polymorphisms in the human and other genomes.

The above data proposes RGENs as providing a platform to use simple and robust RFLP analysis for various sequence variations. With high flexibility in reprogramming target sequence, RGENs can be used to detect various genetic variations (single nucleotide variations, small insertion/deletions, structural variations) such as disease-related recurring mutations, genotypes related to drug-response by a patient and also mutations induced by engineered nucleases in cells. Here, we used RGEN genotyping to detect mutations induced by engineered nucleases in cells and animals. In principle, one could also use RGENs that will specifically detect and cleave naturally-occurring variations and mutations.

Based on the above description, it should be understood by those skilled in the art that various alternatives to the embodiments of the invention described herein may be employed in practicing the invention without departing from the technical idea or essential features of the invention as defined in the following claims. In this regard, the above-described examples are for illustrative purposes only, and the invention is not intended to be limited by these examples. The scope of the present invention should be understood to include all of the modifications or modified form derived from the meaning and scope of the following claims or its equivalent concepts.

### REFERENCES

1. M. Jinek et al., Science 337, 816 (Aug. 17, 2012).
2. H. Kim, E. Um, S. R. Cho, C. Jung, J. S. Kim, Nat Methods 8, 941 (November, 2011).
3. H. J. Kim, H. J. Lee, H. Kim, S. W. Cho, J. S. Kim, Genome Res 19, 1279 (July, 2009).
4. E. E. Perez et al., Nat Biotechnol 26, 808 (July, 2008).
5. J. C. Miller et al., Nat Biotechnol 29, 143 (February, 2011).
6. C. Mussolino et al., Nucleic Acids Res 39, 9283 (November, 2011).
7. J. Cohen, Science 332, 784 (May 13, 2011).
8. V. Pattanayak, C. L. Ramirez, J. K. Joung, D. R. Liu, Nat Methods 8, 765 (September, 2011).
9. R. Gabriel et al., Nat Biotechnol 29, 816 (September, 2011).
10. E. Kim et al., Genome Res, (Apr. 20, 2012).
11. H. J. Lee, J. Kweon, E. Kim, S. Kim, J. S. Kim, Genome Res 22, 539 (March, 2012).
12. H. J. Lee, E. Kim, J. S. Kim, Genome Res 20, 81 (January, 2010).
13. Fu Y, Foden J A, Khayter C, Maeder M L, Reyon D, Joung J K, Sander J D. High-frequency off-target mutagenesis induced by CRISPR-Cas nucleases in human cells. Nat Biotech advance online publication (2013)

1. A method for producing a genome modified embryo, the method comprising:
providing a Cas9/sgRNA complex, comprising a Cas9 protein and a single-guide RNA (sgRNA) in a cell-free buffer, wherein the sgRNA comprises a CRISPR RNA (crRNA) and a transactivating crRNA (tracrRNA); and
introducing the Cas9/sgRNA complex into a non-human embryo, wherein the Cas9/sgRNA complex induces a modification at a target endogenous nucleic acid of the non-human embryo to provide for a genome modified embryo.

2. The method of claim 1, the method further comprising transferring the genome modified embryo into a foster mother.

3. The method of claim 2, the method further comprising allowing the foster mother to produce a F0 animal having the modification at the target endogenous nucleic acid.

4. The method of claim 3, the method further comprising mating the F0 animal with a wild-type non-human animal or another genome-modified non-human animal of the same species as the F0 animal having the modification at the target endogenous nucleic acid to provide a F1 offspring having the modification at the target endogenous nucleic acid.

5. The method of claim 1, wherein the step of introducing the Cas9/sgRNA complex into the non-human embryo comprises introducing the Cas9/sgRNA complex into a pronucleus of the non-human embryo.

6. The method of claim 1, wherein the step of introducing the Cas9/sgRNA complex into the non-human embryo comprises introducing the Cas9/sgRNA complex into a cytoplasm of the non-human embryo.

7. The method of claim 1, wherein the step of introducing the Cas9/sgRNA complex into a non-human embryo comprises microinjection.

8. The method of claim 1, wherein the step of introducing the Cas9/sgRNA complex into a non-human embryo comprises transfection.

9. The method of claim 1, wherein the step of introducing the Cas9/sgRNA complex into a non-human embryo comprises electroporation.

10. The method of claim 1, wherein the non-human embryo is a mammalian embryo.

11. The method of claim 1, wherein the non-human embryo is a livestock embryo.

12. The method of claim 1, wherein the non-human embryo is a mouse embryo.

13. The method of claim 1, wherein the non-human embryo is a fish embryo.

14. The method of claim 1, wherein the non-human embryo is a poultry embryo.


SEQUENCE LISTING (332 sequences)

SEQ ID NO: 1 - DNA, 4107 bp, synthetic construct
  misc_feature 1..4107: note = Description of Artificial Sequence: Synthetic polynucleotide
  misc_feature 1..4107: note = Cas9-coding sequence
  source 1..4107: mol_type = other DNA
atggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactaa

SEQ ID NO: 2 - protein, 21 aa, synthetic construct
  REGION 1..21: note = Description of Artificial Sequence: Synthetic peptide
  REGION 1..21: note = Peptide tag
GGSGPPKKKRKVYPYDVPDYA

SEQ ID NO: 3 - DNA, 34 bp, synthetic construct
  misc_feature 1..34: note = Description of Artificial Sequence: Synthetic oligonucleotide
  misc_feature 1..34: note = F primer for CCR5
  source 1..34: mol_type = other DNA
aattcatgacatcaattattatacatcggaggag

SEQ ID NO: 4 - DNA, 34 bp, synthetic construct
  misc_feature 1..34: note = Description of Artificial Sequence: Synthetic oligonucleotide
  misc_feature 1..34: note = R primer for CCR5
  source 1..34: mol_type = other DNA
gatcctcctccgatgtataataattgatgtcatg

SEQ ID NO: 5 - DNA, 20 bp, synthetic construct
  misc_feature 1..20: note = Description of Artificial Sequence: Synthetic oligonucleotide
  misc_feature 1..20: note = F1 primer for CCR5
  source 1..20: mol_type = other DNA
ctccatggtgctatagagca

SEQ ID NO: 6 - DNA, 21 bp, synthetic construct
  misc_feature 1..21: note = Description of Artificial Sequence: Synthetic oligonucleotide
  misc_feature 1..21: note = F2 primer for CCR5
  source 1..21: mol_type = other DNA
gagccaagctctccatctagt

SEQ ID NO: 7 - DNA, 20 bp, synthetic construct
  misc_feature 1..20: note = Description of Artificial Sequence: Synthetic oligonucleotide
  misc_feature 1..20: note = R primer for CCR5
  source 1..20: mol_type = other DNA
gccctgtcaagagttgacac

SEQ ID NO: 8 - DNA, 20 bp, synthetic construct
  misc_feature 1..20: note = Description of Artificial Sequence: Synthetic oligonucleotide
  misc_feature 1..20: note = F1 primer for C4BPB
  source 1..20: mol_type = other DNA
tatttggctggttgaaaggg

SEQ ID NO: 9 - DNA, 24 bp, synthetic construct
  misc_feature 1..24: note = Description of Artificial Sequence: Synthetic oligonucleotide
  misc_feature 1..24: note = R1 primer for C4BPB
  source 1..24: mol_type = other DNA
aaagtcatgaaataaacacaccca

SEQ ID NO: 10 - DNA, 24 bp, synthetic construct
  misc_feature 1..24: note = Description of Artificial Sequence: Synthetic oligonucleotide
  misc_feature 1..24: note = F2 primer for C4BPB
  source 1..24: mol_type = other DNA
ctgcattgatatggtagtaccatg

SEQ ID NO: 11 - DNA, 21 bp, synthetic construct
  misc_feature 1..21: note = Description of Artificial Sequence: Synthetic oligonucleotide
  misc_feature 1..21: note = R2 primer for C4BPB
  source 1..21: mol_type = other DNA
gctgttcattgcaatggaatg

SEQ ID NO: 12 - DNA, 20 bp, synthetic construct
  misc_feature 1..20: note = Description of Artificial Sequence: Synthetic oligonucleotide
  misc_feature 1..20: note = F1 primer for ADCY5
  source 1..20: mol_type = other DNA
gctcccaccttagtgctctg

SEQ ID NO: 13 - DNA, 20 bp, synthetic construct
  misc_feature 1..20: note = Description of Artificial Sequence: Synthetic oligonucleotide
  misc_feature 1..20: note = R1 primer for ADCY5
  source 1..20: mol_type = other DNA
ggtggcaggaacctgtatgt

SEQ ID NO: 14 - DNA, 21 bp, synthetic construct
  misc_feature 1..21: note = Description of Artificial Sequence: Synthetic oligonucleotide
  misc_feature 1..21: note = F2 primer for ADCY5
  source 1..21: mol_type = other DNA
gtcattggccagagatgtgga

SEQ ID NO: 15 - DNA, 20 bp, synthetic construct
  misc_feature 1..20: note = Description of Artificial Sequence: Synthetic oligonucleotide
  misc_feature 1..20: note = R2 primer for ADCY5
  source 1..20: mol_type = other DNA
gtcccatgacaggcgtgtat

SEQ ID NO: 16 - DNA, 20 bp, synthetic construct
  misc_feature 1..20: note = Description of Artificial Sequence: Synthetic oligonucleotide
  misc_feature 1..20: note = F primer for KCNJ6
  source 1..20: mol_type = other DNA
gcctggccaagtttcagtta

SEQ ID NO: 17 - DNA, 20 bp, synthetic construct
  misc_feature 1..20: note = Description of Artificial Sequence: Synthetic oligonucleotide
  misc_feature 1..20: note = R1 primer for KCNJ6
  source 1..20: mol_type = other DNA
tggagccattggtttgcatc

SEQ ID NO: 18 - DNA, 22 bp, synthetic construct
  misc_feature 1..22: note = Description of Artificial Sequence: Synthetic oligonucleotide
  misc_feature 1..22: note = R2 primer for KCNJ6
  source 1..22: mol_type = other DNA
ccagaactaagccgtttctgac

SEQ ID NO: 19 - DNA, 20 bp, synthetic construct
  misc_feature 1..20: note = Description of Artificial Sequence: Synthetic oligonucleotide
  misc_feature 1..20: note = F1 primer for CNTNAP2
  source 1..20: mol_type = other DNA
atcaccgacaaccagtttcc

SEQ ID NO: 20 - DNA, 20 bp, synthetic construct
  misc_feature 1..20: note = Description of Artificial Sequence: Synthetic oligonucleotide
  misc_feature 1..20: note = F2 primer for CNTNAP2
  source 1..20: mol_type = other DNA
tgcagtgcagactctttcca

SEQ ID NO: 21 - DNA, 20 bp, synthetic construct
  misc_feature 1..20: note = Description of Artificial Sequence: Synthetic oligonucleotide
  misc_feature 1..20: note = R primer for CNTNAP2
  source 1..20: mol_type = other DNA
aaggacacagggcaactgaa

SEQ ID NO: 22 - DNA, 20 bp, synthetic construct
  misc_feature 1..20: note = Description of Artificial Sequence: Synthetic oligonucleotide
  misc_feature 1..20: note = F1 primer for N/A Chr. 5
  source 1..20: mol_type = other DNA
tgtggaacgagtggtgacag

SEQ ID NO: 23 - DNA, 22 bp, synthetic construct
  misc_feature 1..22: note = Description of Artificial Sequence: Synthetic oligonucleotide
  misc_feature 1..22: note = R1 primer for N/A Chr. 5
  source 1..22: mol_type = other DNA
gctggattaggaggcaggattc

SEQ ID NO: 24 - DNA, 22 bp, synthetic construct
  misc_feature 1..22: note = Description of Artificial Sequence: Synthetic oligonucleotide
  misc_feature 1..22: note = F2 primer for N/A Chr. 5
  source 1..22: mol_type = other DNA
gtgctgagaacgcttcatagag

SEQ ID NO: 25 - DNA, 23 bp, synthetic construct
  misc_feature 1..23: note = Description of Artificial Sequence: Synthetic oligonucleotide
  misc_feature 1..23: note = R2 primer for N/A Chr. 5
  source 1..23: mol_type = other DNA
ggaccaaaccacattcttctcac

SEQ ID NO: 26 - DNA, 20 bp, synthetic construct
  misc_feature 1..20: note = Description of Artificial Sequence: Synthetic oligonucleotide
  misc_feature 1..20: note = F primer for deletion
  source 1..20: mol_type = other DNA
ccacatctcgttctcggttt

SEQ ID NO: 27 - DNA, 20 bp, synthetic construct
  misc_feature 1..20: note = Description of Artificial Sequence: Synthetic oligonucleotide
  misc_feature 1..20: note = R primer for deletion
  source 1..20: mol_type = other DNA
tcacaagcccacagatattt

SEQ ID NO: 28 - RNA, 105 nt, synthetic construct
  misc_feature 1..105: note = Description of Artificial Sequence: Synthetic polynucleotide
  misc_feature 1..105: note = sgRNA for CCR5
  source 1..105: mol_type = other RNA
ggtgacatcaattattatacatgttttagagctagaaatagcaagttaaaataaggctagtccgttatcaacttgaaaaagtggcaccgagtcggtgcttttttt

SEQ ID NO: 29 - RNA, 44 nt, synthetic construct
  misc_feature 1..44: note = Description of Artificial Sequence: Synthetic oligonucleotide
  misc_feature 1..44: note = crRNA for CCR5
  source 1..44: mol_type = other RNA
ggtgacatcaattattatacatgttttagagctatgctgttttg

SEQ ID NO: 30 - RNA, 86 nt, synthetic construct
  misc_feature 1..86: note = Description of Artificial Sequence: Synthetic oligonucleotide
  misc_feature 1..86: note = tracrRNA for CCR5
  source 1..86: mol_type = other RNA
ggaaccattcaaaacagcatagcaagttaaaataaggctagtccgttatcaacttgaaaaagtggcaccgagtcggtgcttttttt

SEQ ID NO: 31 - DNA, 86 bp, synthetic construct
  misc_feature 1..86: note = Description of Artificial Sequence: Synthetic oligonucleotide
  misc_feature 1..86: note = Foxn1 #1 sgRNA
  source 1..86: mol_type = other DNA
gaaattaatacgactcactataggcagtctgacgtcacacttccgttttagagctagaaatagcaagttaaaataaggctagtccg

SEQ ID NO: 32 - DNA, 86 bp, synthetic construct
  misc_feature 1..86: note = Description of Artificial Sequence: Synthetic oligonucleotide
  misc_feature 1..86: note = Foxn1 #2 sgRNA
  source 1..86: mol_type = other DNA
gaaattaatacgactcactataggacttccaggctccacccgacgttttagagctagaaatagcaagttaaaataaggctagtccg

SEQ ID NO: 33 - DNA, 86 bp, synthetic construct
  misc_feature 1..86: note = Description of Artificial Sequence: Synthetic oligonucleotide
  misc_feature 1..86: note = Foxn1 #3 sgRNA
  source 1..86: mol_type = other DNA
gaaattaatacgactcactataggccaggctccacccgactggagttttagagctagaaatagcaagttaaaataaggctagtccg

SEQ ID NO: 34 - DNA, 86 bp, synthetic construct
  misc_feature 1..86: note = Description of Artificial Sequence: Synthetic oligonucleotide
  misc_feature 1..86: note = Foxn1 #4 sgRNA
  source 1..86: mol_type = other DNA
gaaattaatacgactcactataggactggagggcgaaccccaaggttttagagctagaaatagcaagttaaaataaggctagtccg

SEQ ID NO: 35 - DNA, 86 bp, synthetic construct
  misc_feature 1..86: note = Description of Artificial Sequence: Synthetic oligonucleotide
  misc_feature 1..86: note = Foxn1 #5 sgRNA
  source 1..86: mol_type = other DNA
gaaattaatacgactcactataggaccccaaggggacctcatgcgttttagagctagaaatagcaagttaaaataaggctagtccg

SEQ ID NO: 36 - DNA, 86 bp, synthetic construct
  misc_feature 1..86: note = Description of Artificial Sequence: Synthetic oligonucleotide
  misc_feature 1..86: note = Prkdc #1 sgRNA
  source 1..86: mol_type = other DNA
gaaattaatacgactcactataggttagttttttccagagacttgttttagagctagaaatagcaagttaaaataaggctagtccg

SEQ ID NO: 37 - DNA, 86 bp, synthetic construct
  misc_feature 1..86: note = Description of Artificial Sequence: Synthetic oligonucleotide
  misc_feature 1..86: note = Prkdc #2 sgRNA
  source 1..86: mol_type = other DNA
gaaattaatacgactcactataggttggtttgcttgtgtttatcgttttagagctagaaatagcaagttaaaataaggctagtccg

SEQ ID NO: 38 - DNA, 86 bp, synthetic construct
  misc_feature 1..86: note = Description of Artificial Sequence: Synthetic oligonucleotide
  misc_feature 1..86: note = Prkdc #3 sgRNA
  source 1..86: mol_type = other DNA
gaaattaatacgactcactataggcacaagcaaaccaaagtctcgttttagagctagaaatagcaagttaaaataaggctagtccg

SEQ ID NO: 39 - DNA, 86 bp, synthetic construct
  misc_feature 1..86: note = Description of Artificial Sequence: Synthetic oligonucleotide
  misc_feature 1..86: note = Prkdc #4 sgRNA
  source 1..86: mol_type = other DNA
gaaattaatacgactcactataggcctcaatgctaagcgacttcgttttagagctagaaatagcaagttaaaataaggctagtccg

SEQ ID NO: 40 - DNA, 29 bp, synthetic construct
  misc_feature 1..29: note = Description of Artificial Sequence: Synthetic oligonucleotide
  misc_feature 1..29: note = F1 primer for Foxn1
  source 1..29: mol_type = other DNA
gtctgtctatcatctcttcccttctctcc

SEQ ID NO: 41 - DNA, 25 bp, synthetic construct
  misc_feature 1..25: note = Description of Artificial Sequence: Synthetic oligonucleotide
  misc_feature 1..25: note = F2 primer for Foxn1
  source 1..25: mol_type = other DNA
tccctaatccgatggctagctccag

SEQ ID NO: 42 - DNA, 23 bp, synthetic construct
  misc_feature 1..23: note = Description of Artificial Sequence: Synthetic oligonucleotide
  misc_feature 1..23: note = R1 primer for Foxn1
  source 1..23: mol_type = other DNA
acgagcagctgaagttagcatgc

SEQ ID NO: 43 - DNA, 32 bp, synthetic construct
  misc_feature 1..32: note = Description of Artificial Sequence: Synthetic oligonucleotide
  misc_feature 1..32: note = R2 primer for Foxn1
  source 1..32: mol_type = other DNA
ctactcaatgctcttagagctaccaggcttgc

SEQ ID NO: 44 - DNA, 20 bp, synthetic construct
  misc_feature 1..20: note = Description of Artificial Sequence: Synthetic oligonucleotide
  misc_feature 1..20: note = F primer for Prkdc
  source 1..20: mol_type = other DNA
gactgttgtggggagggccg

SEQ ID NO: 45 - DNA, 24 bp, synthetic construct
  misc_feature 1..24: note = Description of Artificial Sequence: Synthetic oligonucleotide
  misc_feature 1..24: note = F2 primer for Prkdc
  source 1..24: mol_type = other DNA
gggagggccgaaagtcttattttg

SEQ ID NO: 46 - DNA, 28 bp, synthetic construct
  misc_feature 1..28: note = Description of Artificial Sequence: Synthetic oligonucleotide
  misc_feature 1..28: note = R1 primer for Prkdc
  source 1..28: mol_type = other DNA
cctgaagactgaagttggcagaagtgag

SEQ ID NO: 47 - DNA, 27 bp, synthetic construct
  misc_feature 1..27: note = Description of Artificial Sequence: Synthetic oligonucleotide
  misc_feature 1..27: note = R2 primer for Prkdc
  source 1..27: mol_type = other DNA
ctttagggcttcttctctacaatcacg

SEQ ID NO: 48 - DNA, 38 bp, synthetic construct
  misc_feature 1..38: note = Description of Artificial Sequence: Synthetic oligonucleotide
  misc_feature 1..38: note = F primer for Foxn1
  source 1..38: mol_type = other DNA
ctcggtgtgtagccctgacctcggtgtgtagccctgac

SEQ ID NO: 49 - DNA, 21 bp, synthetic construct
  misc_feature 1..21: note = Description of Artificial Sequence: Synthetic oligonucleotide
  misc_feature 1..21: note = R primer for Foxn1
  source 1..21: mol_type = other DNA
agactggcctggaactcacag

SEQ ID NO: 50 - DNA, 23 bp, synthetic construct
  misc_feature 1..23: note = Description of Artificial Sequence: Synthetic oligonucleotide
  misc_feature 1..23: note = F primer for Foxn1
  source 1..23: mol_type = other DNA
cactaaagcctgtcaggaagccg

SEQ ID NO: 51 - DNA, 21 bp, synthetic construct
  misc_feature 1..21: note = Description of Artificial Sequence: Synthetic oligonucleotide
  misc_feature 1..21: note = R primer for Foxn1
  source 1..21: mol_type = other DNA
ctgtggagagcacacagcagc

SEQ ID NO: 52 - DNA, 19 bp, synthetic construct
  misc_feature 1..19: note = Description of Artificial Sequence: Synthetic oligonucleotide
  misc_feature 1..19: note = F primer for Foxn1
  source 1..19: mol_type = other DNA
gctgcgacctgagaccatg

SEQ ID NO: 53 - DNA, 26 bp, synthetic construct
  misc_feature 1..26: note = Description of Artificial Sequence: Synthetic oligonucleotide
  misc_feature 1..26: note = R primer for Foxn1
  source 1..26: mol_type = other DNA
cttcaatggcttcctgcttaggctac

SEQ ID NO: 54 - DNA, 23 bp, synthetic construct
  misc_feature 1..23: note = Description of Artificial Sequence: Synthetic oligonucleotide
  misc_feature 1..23: note = F primer for Foxn1
  source 1..23: mol_type = other DNA
ggttcagatgaggccatcctttc

SEQ ID NO: 55 - DNA, 24 bp, synthetic construct
  misc_feature 1..24: note = Description of Artificial Sequence: Synthetic oligonucleotide
  misc_feature 1..24: note = R primer for Foxn1
  source 1..24: mol_type = other DNA
cctgatctgcaggcttaacccttg

SEQ ID NO: 56 - DNA, 22 bp, synthetic construct
  misc_feature 1..22: note = Description of Artificial Sequence: Synthetic oligonucleotide
  misc_feature 1..22: note = F primer for Prkdc
  source 1..22: mol_type = other DNA
ctcacctgcacatcacatgtgg

SEQ ID NO: 57 - DNA, 20 bp, synthetic construct
  misc_feature 1..20: note = Description of Artificial Sequence: Synthetic oligonucleotide
  misc_feature 1..20: note = R primer for Prkdc
  source 1..20: mol_type = other DNA
ggcatccaccctatggggtc

SEQ ID NO: 58 - DNA, 25 bp, synthetic construct
  misc_feature 1..25: note = Description of Artificial Sequence: Synthetic oligonucleotide
  misc_feature 1..25: note = F primer for Prkdc
  source 1..25: mol_type = other DNA
gccttgacctagagcttaaagagcc

SEQ ID NO: 59 - DNA, 25 bp, synthetic construct
  misc_feature 1..25: note = Description of Artificial Sequence: Synthetic oligonucleotide
  misc_feature 1..25: note = R primer for Prkdc
  source 1..25: mol_type = other DNA
ggtcttgttagcaggaaggacactg

SEQ ID NO: 60 - DNA, 27 bp, synthetic construct
  misc_feature 1..27: note = Description of Artificial Sequence: Synthetic oligonucleotide
  misc_feature 1..27: note = F primer for Prkdc
  source 1..27: mol_type = other DNA
aaaactctgcttgatgggatatgtggg

SEQ ID NO: 61 - DNA, 26 bp, synthetic construct
  misc_feature 1..26: note = Description of Artificial Sequence: Synthetic oligonucleotide
  misc_feature 1..26: note = R primer for Prkdc
  source 1..26: mol_type = other DNA
ctctcactggttatctgtgctccttc

SEQ ID NO: 62 - DNA, 23 bp, synthetic construct
  misc_feature 1..23: note = Description of Artificial Sequence: Synthetic oligonucleotide
  misc_feature 1..23: note = F primer for Prkdc
  source 1..23: mol_type = other DNA
ggatcaataggtggtgggggatg

SEQ ID NO: 63 - DNA, 27 bp, synthetic construct
  misc_feature 1..27: note = Description of Artificial Sequence: Synthetic oligonucleotide
  misc_feature 1..27: note = R primer for Prkdc
  source 1..27: mol_type = other DNA
gtgaatgacacaatgtgacagcttcag

SEQ ID NO: 64 - DNA, 28 bp, synthetic construct
  misc_feature 1..28: note = Description of Artificial Sequence: Synthetic oligonucleotide
  misc_feature 1..28: note = F primer for Prkdc
  source 1..28: mol_type = other DNA
cacaagacagacctctcaacattcagtc

SEQ ID NO: 65 - DNA, 32 bp, synthetic construct
  misc_feature 1..32: note = Description of Artificial Sequence: Synthetic oligonucleotide
  misc_feature 1..32: note = R primer for Prkdc
  source 1..32: mol_type = other DNA
gtgcatgcatataatccattctgattgctctc

SEQ ID NO: 66 - DNA, 17 bp, synthetic construct
  misc_feature 1..17: note = Description of Artificial Sequence: Synthetic oligonucleotide
  misc_feature 1..17: note = F1 primer for Prkdc
  source 1..17: mol_type = other DNA
gggaggcagaggcaggt

SEQ ID NO: 67 - DNA, 23 bp, synthetic construct
  misc_feature 1..23: note = Description of Artificial Sequence: Synthetic oligonucleotide
  misc_feature 1..23: note = F2 primer for Prkdc
  source 1..23: mol_type = other DNA
ggatctctgtgagtttgaggcca

SEQ ID NO: 68 - DNA, 24 bp, synthetic construct
  misc_feature 1..24: note = Description of Artificial Sequence: Synthetic oligonucleotide
  misc_feature 1..24: note = R1 primer for Prkdc
  source 1..24: mol_type = other DNA
gctccagaactcactcttaggctc

SEQ ID NO: 69 - DNA, 20 bp, synthetic construct
  misc_feature 1..20: note = Description of Artificial Sequence: Synthetic oligonucleotide
  misc_feature 1..20: note = Primer for Foxn1
  source 1..20: mol_type = other DNA
ctactccctccgcagtctga

SEQ ID NO: 70 - DNA, 20 bp, synthetic construct
  misc_feature 1..20: note = Description of Artificial Sequence: Synthetic oligonucleotide
  misc_feature 1..20: note = Primer for Foxn1
  source 1..20: mol_type = other DNA
ccaggcctaggttccaggta

SEQ ID NO: 71 - DNA, 20 bp, synthetic construct
  misc_feature 1..20: note = Description of Artificial Sequence: Synthetic oligonucleotide
  misc_feature 1..20: note = Primer for Prkdc
  source 1..20: mol_type = other DNA
ccccagcattgcagatttcc

SEQ ID NO: 72 - DNA, 23 bp, synthetic construct
  misc_feature 1..23: note = Description of Artificial Sequence: Synthetic oligonucleotide
  misc_feature 1..23: note = Primer for Prkdc
  source 1..23: mol_type = other DNA
agggcttcttctctacaatcacg

SEQ ID NO: 73 - DNA, 86 bp, synthetic construct
  misc_feature 1..86: note = Description of Artificial Sequence: Synthetic oligonucleotide
  misc_feature 1..86: note = BRI1 target 1
  source 1..86: mol_type = other DNA
gaaattaatacgactcactataggtttgaaagatggaagcgcgggttttagagctagaaatagcaagttaaaataaggctagtccg

SEQ ID NO: 74 - DNA, 86 bp, synthetic construct
  misc_feature 1..86: note = Description of Artificial Sequence: Synthetic oligonucleotide
  misc_feature 1..86: note = BRI1 target 2
  source 1..86: mol_type = other DNA
gaaattaatacgactcactataggtgaaactaaactggtccacagttttagagctagaaatagcaagttaaaataaggctagtccg

SEQ ID NO: 75 - DNA, 64 bp, synthetic construct
  misc_feature 1..64: note = Description of Artificial Sequence: Synthetic oligonucleotide
  misc_feature 1..64: note = Universal
  source 1..64: mol_type = other DNA
aaaaaagcaccgactcggtgccactttttcaagttgataacggactagccttattttaacttgc

SEQ ID NO: 76 - DNA, 65 bp, synthetic construct
  misc_feature 1..65: note = Description of Artificial Sequence: Synthetic oligonucleotide
  misc_feature 1..65: note = Templates for crRNA
  misc_difference 25..44: note = modified_base - a, c, t, g, unknown or other
  source 1..65: mol_type = other DNA
gaaattaatacgactcactataggnnnnnnnnnnnnnnnnnnnngttttagagctatgctgtttt

SEQ ID NO: 77 - DNA, 67 bp, synthetic construct
  misc_feature 1..67: note = Description of Artificial Sequence: Synthetic oligonucleotide
  misc_feature 1..67: note = tracrRNA
  source 1..67: mol_type = other DNA
gaaattaatacgactcactataggaaccattcaaaacagcatagcaagttaaaataaggctagtccg

SEQ ID NO: 78 - DNA, 69 bp, synthetic construct
  misc_feature 1..69: note = Description of Artificial Sequence: Synthetic oligonucleotide
  misc_feature 1..69: note = tracrRNA
  source 1..69: mol_type = other DNA
aaaaaaagcaccgactcggtgccactttttcaagttgataacggactagccttattttaacttgctatg

SEQ ID NO: 79 - DNA, 20 bp, synthetic construct
  misc_feature 1..20: note = Description of Artificial Sequence: Synthetic oligonucleotide
  misc_feature 1..20: note = Primer
  source 1..20: mol_type = other DNA
ctccatggtgctatagagca

SEQ ID NO: 80 - DNA, 21 bp, synthetic construct
  misc_feature 1..21: note = Description of Artificial Sequence: Synthetic oligonucleotide
  misc_feature 1..21: note = Primer
  source 1..21: mol_type = other DNA
gagccaagctctccatctagt

SEQ ID NO: 81 - DNA, 20 bp, synthetic construct
  misc_feature 1..20: note = Description of Artificial Sequence: Synthetic oligonucleotide
  misc_feature 1..20: note = Primer
  source 1..20: mol_type = other DNA
gccctgtcaagagttgacac

SEQ ID NO: 82 - DNA, 22 bp, synthetic construct
  misc_feature 1..22: note = Description of Artificial Sequence: Synthetic oligonucleotide
  misc_feature 1..22: note = Primer
  source 1..22: mol_type = other DNA
gcacagggtggaacaagatgga

SEQ ID NO: 83 - DNA, 24 bp, synthetic construct
  misc_feature 1..24: note = Description of Artificial Sequence: Synthetic oligonucleotide
  misc_feature 1..24: note = Primer
  source 1..24: mol_type = other DNA
gccaggtacctatcgattgtcagg

SEQ ID NO: 84 - DNA, 21 bp, synthetic construct
  misc_feature 1..21: note = Description of Artificial Sequence: Synthetic oligonucleotide
  misc_feature 1..21: note = Primer
  source 1..21: mol_type = other DNA
gagccaagctctccatctagt

SEQ ID NO: 85 - DNA, 20 bp, synthetic construct
  misc_feature 1..20: note = Description of Artificial Sequence: Synthetic oligonucleotide
  misc_feature 1..20: note = Primer
  source 1..20: mol_type = other DNA
actctgactgggtcaccagc

SEQ ID NO: 86 - DNA, 20 bp, synthetic construct
  misc_feature 1..20: note = Description of Artificial Sequence: Synthetic oligonucleotide
  misc_feature 1..20: note = Primer
  source 1..20: mol_type = other DNA
tatttggctggttgaaaggg

SEQ ID NO: 87 - DNA, 24 bp, synthetic construct
  misc_feature 1..24: note = Description of Artificial Sequence: Synthetic oligonucleotide
  misc_feature 1..24: note = Primer
  source 1..24: mol_type = other DNA
aaagtcatgaaataaacacaccca

SEQ ID NO: 88 - DNA, 24 bp, synthetic construct
  misc_feature 1..24: note = Description of Artificial Sequence: Synthetic oligonucleotide
  misc_feature 1..24: note = Primer
  source 1..24: mol_type = other DNA
ctgcattgatatggtagtaccatg

SEQ ID NO: 89 - DNA, 21 bp, synthetic construct
  misc_feature 1..21: note = Description of Artificial Sequence: Synthetic oligonucleotide
  misc_feature 1..21: note = Primer
  source 1..21: mol_type = other DNA
gctgttcattgcaatggaatg

SEQ ID NO: 90 - DNA, 22 bp, synthetic construct
  misc_feature 1..22: note = Description of Artificial Sequence: Synthetic oligonucleotide
  misc_feature 1..22: note = Primer
  source 1..22: mol_type = other DNA
atggagttggacatggccatgg

SEQ ID NO: 91 - DNA, 28 bp, synthetic construct
  misc_feature 1..28: note = Description of Artificial Sequence: Synthetic oligonucleotide
  misc_feature 1..28: note = Primer
  source 1..28: mol_type = other DNA
actcactatccacagttcagcatttacc

SEQ ID NO: 92 - DNA, 23 bp, synthetic construct
  misc_feature 1..23: note = Description of Artificial Sequence: Synthetic oligonucleotide
  misc_feature 1..23: note = Primer
  source 1..23: mol_type = other DNA
tggagatagctgtcagcaacttt

SEQ ID NO: 93 - DNA, 29 bp, synthetic construct
  misc_feature 1..29: note = Description of Artificial Sequence: Synthetic oligonucleotide
  misc_feature 1..29: note = Primer
  source 1..29: mol_type = other DNA
caacaaagcaaaggtaaagttggtaatag

SEQ ID NO: 94 - DNA, 25 bp, synthetic construct
  misc_feature 1..25: note = Description of Artificial Sequence: Synthetic oligonucleotide
  misc_feature 1..25: note = Primer
  source 1..25: mol_type = other DNA
ggtttcaggagatgtgttacaaggc

SEQ ID NO: 95 - DNA, 27 bp, synthetic construct
  misc_feature 1..27: note = Description of Artificial Sequence: Synthetic oligonucleotide
  misc_feature 1..27: note = Primer
  source 1..27: mol_type = other DNA
gattgtgcaattcctatgcaatcggtc

SEQ ID NO: 96 - DNA, 25 bp, synthetic construct
  misc_feature 1..25: note = Description of Artificial Sequence: Synthetic oligonucleotide
  misc_feature 1..25: note = Primer
  source 1..25: mol_type = other DNA
cactgggtacttaatctgtagcctc

SEQ ID NO: 97 - DNA, 23 bp, synthetic construct
  misc_feature 1..23: note = Description of Artificial Sequence: Synthetic oligonucleotide
  misc_feature 1..23: note = Primer
  source 1..23: mol_type = other DNA
ggttccaagtcattcccagtagc

SEQ ID NO: 98 - DNA, 30 bp, synthetic construct
  misc_feature 1..30: note = Description of Artificial Sequence: Synthetic oligonucleotide
  misc_feature 1..30: note = Primer
  source 1..30: mol_type = other DNA
catcactgcagttgtaggttataactatcc

SEQ ID NO: 99 - DNA, 26 bp, synthetic construct
  misc_feature 1..26: note = Description of Artificial Sequence: Synthetic oligonucleotide
  misc_feature 1..26: note = Primer
  source 1..26: mol_type = other DNA
ttgaaaaccacagatctggttgaacc

SEQ ID NO: 100 - DNA, 22 bp, synthetic construct
  misc_feature 1..22: note = Description of Artificial Sequence: Synthetic oligonucleotide
  misc_feature 1..22: note = Primer
  source 1..22: mol_type = other DNA
ggagtgccaagagaatatctgg

SEQ ID NO: 101 - DNA, 32 bp, synthetic construct
  misc_feature 1..32: note = Description of Artificial Sequence: Synthetic oligonucleotide
  misc_feature 1..32: note = Primer
  source 1..32: mol_type = other DNA
ctgaaactggtttcaaaatattcgttttaagg

SEQ ID NO: 102 - DNA, 22 bp, synthetic construct
  misc_feature 1..22: note = Description of Artificial Sequence: Synthetic oligonucleotide
  misc_feature 1..22: note = Primer
  source 1..22: mol_type = other DNA
gctctgtatgccctgtagtagg

SEQ ID NO: 103 - DNA, 22 bp, synthetic construct
  misc_feature 1..22: note = Description of Artificial Sequence: Synthetic oligonucleotide
  misc_feature 1..22: note = Primer
  source 1..22: mol_type = other DNA
tttgcatctgaccttacctttg

SEQ ID NO: 104 - DNA, 23 bp, synthetic construct
  misc_feature 1..23: note = Description of Artificial Sequence: Synthetic oligonucleotide
  misc_feature 1..23: note = Target sequence of RGEN
  source 1..23: mol_type = other DNA
aatgaccactacatcctcaaggg

SEQ ID NO: 105 - DNA, 23 bp, synthetic construct
  misc_feature 1..23: note = Description of Artificial Sequence: Synthetic oligonucleotide
  misc_feature 1..23: note = Target sequence of RGEN
  source 1..23: mol_type = other DNA
agatgatgtctcatcatcagagg

SEQ ID NO: 106 - DNA, 4170 bp, synthetic construct
  misc_feature 1..4170: note = Description of Artificial Sequence: Synthetic polynucleotide
  misc_feature 1..4170: note = Cas9-coding sequence in p3s-Cas9HC (humanized, C-term tagging, human cell experiments)
  source 1..4170: mol_type = other DNA
atggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgacggcggctccggacctccaaagaaaaagagaaaagtatacccctacgacgtgcccgactacgcctaa

SEQ ID NO: 107 - DNA, 4194 bp, synthetic construct
  misc_feature 1..4194: note = Description of Artificial Sequence: Synthetic polynucleotide
  misc_feature 1..4194: note = Cas9 coding sequence in p3s-Cas9HN (humanized codon, N-term tagging (underlined), human cell experiments)
  source 1..4194: mol_type = other DNA
atggtgtacccctacgacgtgcccgactacgccgaattgcctccaaaaaagaagagaaaggtagggatccgaattcccggggaaaaaccggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactaa

SEQ ID NO: 108 - DNA, 4107 bp, synthetic construct
  misc_feature 1..4107: note = Description of Artificial Sequence: Synthetic polynucleotide
  misc_feature 1..4107: note = Cas9-coding sequence in Streptococcus pyogenes
  source 1..4107: mol_type = other DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaa

SEQ ID NO: 109 - protein, 1368 aa, synthetic construct [sequence omitted]

SEQ ID NO: 110 - DNA, 4221 bp, synthetic construct
  misc_feature 1..4221: note = Description of Artificial Sequence: Synthetic polynucleotide
  misc_feature 1..4221: note = Cas9-coding sequence in pET-Cas9N3T for the production of recombinant Cas9 protein in E. coli (humanized codon; hexa-His-tag and a nuclear localization signal at the N terminus)
  source 1..4221: mol_type = other DNA
atgggcagcagccatcatcatcatcatcatgtgtacccctacgacgtgcccgactacgccgaattgcctccaaaaaagaagagaaaggtagggatcgagaacctgtacttccagggcgacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactaa

SEQ ID NO: 111 - protein, 1406 aa, synthetic construct
  REGION 1..1406: note = Description of Artificial Sequence: Synthetic polypeptide
  REGION 1..1406: note = Amino acid sequence of Cas9 (pET-Cas9N3T)
MGSSHHHHHHVYPYDVPDYAELPPKKKRKVGIENLYFQGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 112 - DNA, 34 bp, Homo sapiens
  source 1..34: mol_type = unassigned DNA
caatctatgacatcaattattatacatcggagcc

SEQ ID NO: 113 - RNA, 64 nt, synthetic construct
  misc_feature 1..64: note = Description of Artificial Sequence: Synthetic oligonucleotide
  source 1..64: mol_type = other RNA
ggtgacatcaattattatacatgttttagagctagaaatagcaagttaaaataaggctagtccg

SEQ ID NO: 114 - DNA, 49 bp, synthetic construct
  misc_feature 1..49: note = Description of Artificial Sequence: Synthetic oligonucleotide
  source 1..49: mol_type = other DNA
caatctatgacatcaattattatacatcggagccctgccaaaaaatcaa

SEQ ID NO: 115 - DNA, 50 bp, synthetic construct
  misc_feature 1..50: note = Description of Artificial Sequence: Synthetic oligonucleotide
  source 1..50: mol_type = other DNA
caatctatgacatcaattattataacatcggagccctgccaaaaaatcaa

SEQ ID NO: 116 - DNA, 36 bp, synthetic construct
  misc_feature 1..36: note = Description of Artificial Sequence: Synthetic oligonucleotide
  source 1..36: mol_type = other DNA
caatctatgacatcaattattatgccaaaaaatcaa

SEQ ID NO: 117 - DNA, 35 bp, synthetic construct
  misc_feature 1..35: note = Description of Artificial Sequence: Synthetic oligonucleotide
  source 1..35: mol_type = other DNA
caatctatgacatcggagccctgccaaaaaatcaa

SEQ ID NO: 118 - DNA, 31 bp, synthetic construct
  misc_feature 1..31: note = Description of Artificial Sequence: Synthetic oligonucleotide
  source 1..31: mol_type = other DNA
caatctatgacatgccctgccaaaaaatcaa

SEQ ID NO: 119 - DNA, 30 bp, synthetic construct
  misc_feature 1..30: note = Description of Artificial Sequence: Synthetic oligonucleotide
  source 1..30: mol_type = other DNA
caatctatgacatcaattattataaatcaa

SEQ ID NO: 120 - DNA, 25 bp, synthetic construct
  misc_feature 1..25: note = Description of Artificial Sequence: Synthetic oligonucleotide
  source 1..25: mol_type = other DNA
caatctatgacatccaaaaaatcaa

SEQ ID NO: 121 - DNA, 19 bp, synthetic construct
  misc_feature 1..19: note = Description of Artificial Sequence: Synthetic oligonucleotide
  source 1..19: mol_type = other DNA
caatctatgacaaaatcaa

SEQ ID NO: 122 - DNA, 46 bp, Homo sapiens
  source 1..46: mol_type = unassigned DNA
tatgtgcaatgaccactacatcctcaagggcagcaatcggagccag

SEQ ID NO: 123 - DNA, 47 bp, synthetic construct
  misc_feature 1..47: note = Description of Artificial Sequence: Synthetic oligonucleotide
  source 1..47: mol_type = other DNA
tatgtgcaatgaccactacatccttcaagggcagcaatcggagccag

SEQ ID NO: 124 - DNA, 48 bp, synthetic construct
  misc_feature 1..48: note = Description of Artificial Sequence: Synthetic oligonucleotide
  source 1..48: mol_type = other DNA
tatgtgcaatgaccactacatcctctcaagggcagcaatcggagccag

SEQ ID NO: 125 - DNA, 18 bp, synthetic construct
  misc_feature 1..18: note = Description of Artificial Sequence: Synthetic oligonucleotide
  source 1..18: mol_type = other DNA
tatgtgcaatggagccag

SEQ ID NO: 126 - DNA, 13 bp, synthetic construct
  misc_feature 1..13: note = Description of Artificial Sequence: Synthetic oligonucleotide
  source 1..13: mol_type = other DNA
tatgtgcaatgac

SEQ ID NO: 127 - DNA, 23 bp, Homo sapiens
  source 1..23: mol_type = unassigned DNA
tgacatcaattattatacatcgg

SEQ ID NO: 128 - DNA, 23 bp, Homo sapiens
  source 1..23: mol_type = unassigned DNA
tgacatcaattattatagatgga

SEQ ID NO: 129 - DNA, 23 bp, Homo sapiens
  source 1..23: mol_type = unassigned DNA
tgacatcacttattatgcatggg

SEQ ID NO: 130 - DNA, 23 bp, Homo sapiens
  source 1..23: mol_type = unassigned DNA
tgacataaattattctacatggg

SEQ ID NO: 131 - DNA, 23 bp, Homo sapiens
  source 1..23: mol_type = unassigned DNA
tgaaatcaattatcatagatcgg

SEQ ID NO: 132 - DNA, 23 bp, Homo sapiens
  source 1..23: mol_type = unassigned DNA
ccaggctccacccgactggaggg

SEQ ID NO: 133 - RNA, 106 nt, synthetic construct
  misc_feature 1..106: note = Description of Artificial Sequence: Synthetic polynucleotide
  source 1..106: mol_type = other RNA
ggccaggctccacccgactggagttttagagctagaaatagcaagttaaaataaggctagtccgttatcaacttgaaaaagtggcaccgagtcggtgctttttttt

SEQ ID NO: 134 - DNA, 53 bp, Homo sapiens
  source 1..53: mol_type = unassigned DNA
acttccaggctccacccgactggagggcgaaccccaaggggacctcatgcagg

SEQ ID NO: 135 - DNA, 13 bp, synthetic construct
  misc_feature 1..13: note = Description of Artificial Sequence: Synthetic oligonucleotide
  source 1..13: mol_type = other DNA
acttccaggctcc

SEQ ID NO: 136 - DNA, 30 bp, synthetic construct
  misc_feature 1..30: note = Description of Artificial Sequence: Synthetic oligonucleotide
  source 1..30: mol_type = other DNA
acttccaggctccacccgacctcatgcagg

SEQ ID NO: 137 - DNA, 36 bp, synthetic construct
  misc_feature 1..36: note = Description of Artificial Sequence: Synthetic oligonucleotide
  source 1..36: mol_type = other DNA
acttccaggctccaccccaaggggacctcatgcagg

SEQ ID NO: 138 - DNA, 54 bp, synthetic construct
  misc_feature 1..54: note = Description of Artificial Sequence: Synthetic oligonucleotide
  source 1..54: mol_type = other DNA
acttccaggctccacccgacttggagggcgaaccccaaggggacctcatgcagg

SEQ ID NO: 139 - DNA, 43 bp, synthetic construct
  misc_feature 1..43: note = Description of Artificial Sequence: Synthetic oligonucleotide
  source 1..43: mol_type = other DNA
acttccaggctccacccgaaccccaaggggacctcatgcaggg

SEQ ID NO: 140 - DNA, 47 bp, synthetic construct
  misc_feature 1..47: note = Description of Artificial Sequence: Synthetic oligonucleotide
  source 1..47: mol_type = other DNA
acttccaggctccacccgactcactatcttctgggctcctccatgtc

SEQ ID NO: 141 - DNA, 45 bp, synthetic construct
  misc_feature 1..45: note = Description of Artificial Sequence: Synthetic oligonucleotide
  source 1..45: mol_type = other DNA
acttccaggctccacccgacgaaccccaaggggacctcatgcagg

SEQ ID NO: 142 - protein, 18 aa, Homo sapiens
LPGSTRLEGEPQGDLMQA

SEQ ID NO: 143 - DNA, 57 bp, Homo sapiens
  source 1..57: mol_type = unassigned DNA
  CDS 2..55
acttccaggctccacccgactggagggcgaaccccaaggggacctcatgcaggctcc

SEQ ID NO: 144 - DNA, 46 bp, synthetic construct
  misc_feature 1..46: note = Description of Artificial Sequence: Synthetic oligonucleotide
  source 1..46: mol_type = other DNA
acttccaggctccacccgaaccccaaggggacctcatgcaggctcc

SEQ ID NO: 145 - DNA, 43 bp, synthetic construct
  misc_feature 1..43: note = Description of Artificial Sequence: Synthetic oligonucleotide
  source 1..43: mol_type = other DNA
acttccaggctccacccgaaccccaaggggacctcatgcaggc

SEQ ID NO: 146 - DNA, 20 bp, synthetic construct
  misc_feature 1..20: note = Description of Artificial Sequence: Synthetic oligonucleotide
  source 1..20: mol_type = other DNA
acttccaggctccacccgac

SEQ ID NO: 147 - DNA, 40 bp, synthetic construct
  misc_feature 1..40: note = Description of Artificial Sequence: Synthetic oligonucleotide
  source 1..40: mol_type = other DNA
acttccaggctccaccccaaggggacctcatgcaggctcc

SEQ ID NO: 148 - DNA, 58 bp, synthetic construct
  misc_feature 1..58: note = Description of Artificial Sequence: Synthetic oligonucleotide
  source 1..58: mol_type = other DNA
acttccaggctccacccgacttggagggcgaaccccaaggggacctcatgcaggctcc

SEQ ID NO: 149 - DNA, 46 bp, synthetic construct
  misc_feature 1..46: note = Description of Artificial Sequence: Synthetic oligonucleotide
  source 1..46: mol_type = other DNA
acttccaggctccaggcgaaccccaaggggacctcatgcaggctcc

SEQ ID NO: 150 - DNA, 32 bp, synthetic construct
  misc_feature 1..32: note = Description of Artificial Sequence: Synthetic oligonucleotide
  source 1..32: mol_type = other DNA
ggcgaaccccaaggggacctcatgcaggctcc

SEQ ID NO: 151 - DNA, 32 bp, synthetic construct
  misc_feature 1..32: note = Description of Artificial Sequence: Synthetic oligonucleotide
  source 1..32: mol_type = other DNA
acttccaggcaaggggacctcatgcaggctcc

SEQ ID NO: 152 - DNA, 33 bp, synthetic construct
  misc_feature 1..33: note = Description of Artificial Sequence: Synthetic oligonucleotide
  source 1..33: mol_type = other DNA
acttccaggctaaggggacctcatgcaggctcc

SEQ ID NO: 153 - DNA, 52 bp, Homo sapiens
  source 1..52: mol_type = unassigned DNA
acttccaggctccacccgactggagggcgaaccccaaggggacctcatgcag

SEQ ID NO: 154 - DNA, 34 bp, synthetic construct
  misc_feature 1..34: note = Description of Artificial Sequence: Synthetic oligonucleotide
  source 1..34: mol_type = other DNA
acttccaggcgaaccccaaggggacctcatgcag

SEQ ID NO: 155 - DNA, 32 bp, synthetic construct
  misc_feature 1..32: note = Description of Artificial Sequence: Synthetic oligonucleotide
  source 1..32: mol_type = other DNA
acttccaggctccacaaggggacctcatgcag

SEQ ID NO: 156 - DNA, 34 bp, synthetic construct
  misc_feature 1..34: note = Description of Artificial Sequence: Synthetic oligonucleotide
  source 1..34: mol_type = other DNA
acttccaggctccacccaaggggacctcatgccc

SEQ ID NO: 157 - DNA, 35 bp, synthetic construct
  misc_feature 1..35: note = Description of Artificial Sequence: Synthetic oligonucleotide
  source 1..35: mol_type = other DNA
acttccaggctccaccccaaggggacctcatgcag

SEQ ID NO: 158 - DNA, 41 bp, synthetic construct
  misc_feature 1..41: note = Description of Artificial Sequence: Synthetic oligonucleotide
  source 1..41: mol_type = other DNA
acttccaggctccacccgaaccccaaggggacctcatgcag

SEQ ID NO: 159 - DNA, 50 bp, synthetic construct
  misc_feature 1..50: note = Description of Artificial Sequence: Synthetic oligonucleotide
  source 1..50: mol_type = other DNA
acttccaggctccacccgaaggagggcgaaccccaaggggacctcatgca

SEQ ID NO: 160 - DNA, 50 bp, synthetic construct
  misc_feature 1..50: note = Description of Artificial Sequence: Synthetic oligonucleotide
  source 1..50: mol_type = other DNA
acttccaggctccacccgactagggcgaaccccaaggggacctcatgcag

SEQ ID NO: 161 - DNA, 52 bp, synthetic construct
  misc_feature 1..52: note = Description of Artificial Sequence: Synthetic oligonucleotide
  source 1..52: mol_type = other DNA
acttccaggctccacccgactgggagggcgaaccccaaggggacctcatgca

SEQ ID NO: 162 - DNA, 52 bp, synthetic construct
  misc_feature 1..52: note = Description of Artificial Sequence: Synthetic oligonucleotide
  source 1..52: mol_type = other DNA
acttccaggctccacccgacttggagggcgaaccccaaggggacctcatgca

SEQ ID NO: 163 - DNA, 46 bp, synthetic construct
  misc_feature 1..46: note = Description of Artificial Sequence: Synthetic oligonucleotide
  source 1..46: mol_type = other DNA
acttccaggctccacccgaggcgaaccccaaggggacctcatgcag

SEQ ID NO: 164 - DNA, 47 bp, synthetic construct
  misc_feature 1..47: note = Description of Artificial Sequence: Synthetic oligonucleotide
  source 1..47: mol_type = other DNA
acttccaggctccacccgagggcgaaccccaaggggacctcatgcag

SEQ ID NO: 165 - DNA, 24 bp, synthetic construct
  misc_feature 1..24: note = Description of Artificial Sequence: Synthetic oligonucleotide
  source 1..24: mol_type = other DNA
acttccaggctccacctcatgcag

SEQ ID NO: 166 - DNA, 29 bp, synthetic construct
  misc_feature 1..29: note = Description of Artificial Sequence: Synthetic oligonucleotide
  source 1..29: mol_type = other DNA
agggcgaaccccaaggggacctcatgcag

SEQ ID NO: 167 - DNA, 45 bp, synthetic construct
  misc_feature 1..45: note = Description of Artificial Sequence: Synthetic oligonucleotide
  source 1..45: mol_type = other DNA
caatctatgacatcaattattatcggagccctgccaaaaaatcaa

SEQ ID NO: 168 - DNA, 45 bp, synthetic construct
  misc_feature 1..45: note = Description of Artificial Sequence: Synthetic oligonucleotide
  source 1..45: mol_type = other DNA
caatctatgacatcaattatcatcggagccctgccaaaaaatcaa

SEQ ID NO: 169 - DNA, 42 bp, synthetic construct
  misc_feature 1..42: note = Description of Artificial Sequence: Synthetic oligonucleotide
  source 1..42: mol_type = other DNA
caatctatgacatcaattatcggagccctgccaaaaaatcaa

SEQ ID NO: 170 - DNA, 48 bp, synthetic construct
  misc_feature 1..48: note = Description of Artificial Sequence: Synthetic oligonucleotide
  source 1..48: mol_type = other DNA
caatctatgacatcaattattatcatcggagccctgccaaaaaatcaa

SEQ ID NO: 171 - DNA, 33 bp, synthetic construct
  misc_feature 1..33: note = Description of Artificial Sequence: Synthetic oligonucleotide
  source 1..33: mol_type = other DNA
caatctatgacaagagccctgccaaaaaatcaa

SEQ ID NO: 172 - DNA, 52 bp, Homo sapiens
  source 1..52: mol_type = unassigned DNA
ttctcaaggcagcatcatacttcccccacggtgggacagctgccctccctgg

SEQ ID NO: 173 - DNA, 46 bp, synthetic construct
  misc_feature 1..46: note = Description of Artificial Sequence: Synthetic oligonucleotide
  source 1..46: mol_type = other DNA
ttctcaaggcagcatcatacttccctgggacagctgccctccctgg

SEQ ID NO: 174 - DNA, 49 bp, synthetic construct
  misc_feature 1..49: note = Description of Artificial Sequence: Synthetic oligonucleotide
  source 1..49: mol_type = other DNA
ttctcaaggcagcatcatacttccacggtgggacagctgccctccctgg

SEQ ID NO: 175 - DNA, 25 bp, synthetic construct
  misc_feature 1..25: note = Description of Artificial Sequence: Synthetic oligonucleotide
  source 1..25: mol_type = other DNA
ttctcaaggcagctgccctccctgg

SEQ ID NO: 176 - DNA, 32 bp, synthetic construct
  misc_feature 1..32: note = Description of Artificial Sequence: Synthetic oligonucleotide
  source 1..32: mol_type = other DNA
ttctcaaggcagcatcatacttccctccctgg

SEQ ID NO: 177 - DNA, 264 bp, Homo sapiens
  misc_difference 38..227: note = modified_base - a, c, t, g, unknown or other
  source 1..264: mol_type = unassigned DNA
acaaagcgattttgaaagatggaagcgcggtggctatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggggtgaaactaaactggtccacacggcggaagattg

SEQ ID NO: 178 - DNA, 257 bp, synthetic construct
  misc_feature 1..257: note = Description of Artificial Sequence: Synthetic polynucleotide
  misc_difference 38..227: note = modified_base - a, c, t, g, unknown or other
  source 1..257: mol_type = other DNA
acaaagcgattttgaaagatggaagcgcggtggctatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggggtgaaactaaaacacggcggaagattg

SEQ ID NO: 179 - DNA, 43 bp, synthetic construct
  misc_feature 1..43: note = Description of Artificial Sequence: Synthetic oligonucleotide
  source 1..43: mol_type = other DNA
acaaagcgattttgaaagatggaagcgacacggcggaagattg

SEQ ID NO: 180 - DNA, 44 bp, synthetic construct
  misc_feature 1..44: note = Description of Artificial Sequence: Synthetic oligonucleotide
  source 1..44: mol_type = other DNA
acaaagcgattttgaaagatggaagcgcacacggcggaagattg

SEQ ID NO: 181 - DNA, 106 bp, synthetic construct
  misc_feature 1..106: note = Description of Artificial Sequence: Synthetic polynucleotide
  source 1..106: mol_type = other DNA
acaaagcgattttgaaagatggaagcgaaatagcaagttaaaataaggctagtccgttatcaacttgaaaaagtggcaccgagtcggtgcacacggcggaagattg

SEQ ID NO: 182 - DNA, 23 bp, synthetic construct
  misc_feature 1..23: note = Description of Artificial Sequence: Synthetic oligonucleotide
  source 1..23: mol_type = other DNA
gggtggggggagtttgctcctgg

SEQ ID NO: 183 - DNA, 23 bp, synthetic construct
  misc_feature 1..23: note = Description of Artificial Sequence: Synthetic oligonucleotide
  source 1..23: mol_type = other DNA
ggatggagggagtttgctcctgg

SEQ ID NO: 184 - DNA, 23 bp, synthetic construct
  misc_feature 1..23: note = Description of Artificial Sequence: Synthetic oligonucleotide
  source 1..23: mol_type = other DNA
ggggaggggaagtttgctcctgg

SEQ ID NO: 185 - DNA, 23 bp, synthetic construct
  misc_feature 1..23: note = Description of Artificial Sequence: Synthetic oligonucleotide
  source 1..23: mol_type = other DNA
gaccccctccaccccgcctccgg

SEQ ID NO: 186 - DNA, 23 bp, synthetic construct
  misc_feature 1..23: note = Description of Artificial Sequence: Synthetic oligonucleotide
  source 1..23: mol_type = other DNA
gaccccccccaccccgcccccgg

SEQ ID NO: 187 - DNA, 23 bp, synthetic construct
  misc_feature 1..23: note = Description of Artificial Sequence: Synthetic oligonucleotide
  source 1..23: mol_type = other DNA
gcccccacccaccccgcctctgg

SEQ ID NO: 188 - DNA, 23 bp, synthetic construct
  misc_feature 1..23: note = Description of Artificial Sequence: Synthetic oligonucleotide
  source 1..23: mol_type = other DNA
ctccccacccaccccgcctcagg

SEQ ID NO: 189 - DNA, 23 bp, synthetic construct
  misc_feature 1..23: note = Description of Artificial Sequence: Synthetic oligonucleotide
  source 1..23: mol_type = other DNA
ggtgagtgagtgtgtgcgtgtgg

SEQ ID NO: 190 - DNA, 23 bp, synthetic construct
  misc_feature 1..23: note = Description of Artificial Sequence: Synthetic oligonucleotide
  source 1..23: mol_type = other DNA
tgtgggtgagtgtgtgcgtgagg

SEQ ID NO: 191 - DNA, 23 bp, synthetic construct
  misc_feature 1..23: note = Description of Artificial Sequence: Synthetic oligonucleotide
  source 1..23: mol_type = other DNA
gagtccgagcagaagaagaaggg

SEQ ID NO: 192 - DNA, 23 bp, synthetic construct
  misc_feature 1..23: note = Description of Artificial Sequence: Synthetic oligonucleotide
  source 1..23: mol_type = other DNA
gagttagagcagaagaagaaagg

SEQ ID NO: 193 - DNA, 23 bp, synthetic construct
  misc_feature 1..23: note = Description of Artificial Sequence: Synthetic oligonucleotide
  source 1..23: mol_type = other DNA
gaacctgagctgctctgacgcgg

SEQ ID NO: 194 - DNA, 23 bp, synthetic construct
  misc_feature 1..23: note = Description of Artificial Sequence: Synthetic oligonucleotide
  source 1..23: mol_type = other DNA
ttggcagggggtgggagggaagg

SEQ ID NO: 195 - DNA, 23 bp, synthetic construct
  misc_feature 1..23: note = Description of Artificial Sequence: Synthetic oligonucleotide
  source 1..23: mol_type = other DNA
gggagggagagcttggcaggggg

SEQ ID NO: 196 - DNA, 23 bp, synthetic construct
  misc_feature 1..23: note = Description of Artificial Sequence: Synthetic oligonucleotide
  source 1..23: mol_type = other DNA
gatggagccagagaggatcctgg

SEQ ID NO: 197 - DNA, 23 bp, synthetic construct
  misc_feature 1..23: note = Description of Artificial Sequence: Synthetic oligonucleotide
  source 1..23: mol_type = other DNA
cctgccaagctctccctcccagg

SEQ ID NO: 198 - DNA, 23 bp, synthetic construct
  misc_feature 1..23: note = Description of Artificial Sequence: Synthetic oligonucleotide
  source 1..23: mol_type = other DNA
ctccctcccaggatcctctctgg

SEQ ID NO: 199 - DNA, 23 bp, synthetic construct
  misc_feature 1..23: note = Description of Artificial Sequence: Synthetic oligonucleotide
  source 1..23: mol_type = other DNA
cctctaaggtttgcttacgatgg

SEQ ID NO: 200 - DNA, 23 bp, synthetic construct
  misc_feature 1..23: note = Description of Artificial Sequence: Synthetic oligonucleotide
  source 1..23: mol_type = other DNA
ggttctggcaaggagagagatgg

SEQ ID NO: 201 - DNA, 23 bp, synthetic construct
  misc_feature 1..23: note = Description of Artificial Sequence: Synthetic oligonucleotide
  source 1..23: mol_type = other DNA
tctaacccccacctcctgttagg

SEQ ID NO: 202 - DNA, 23 bp, synthetic construct
  misc_feature 1..23: note = Description of Artificial Sequence: Synthetic oligonucleotide
  source 1..23: mol_type = other DNA
ttggcagggggtgggagggatgg

SEQ ID NO: 203 - DNA, 23 bp, synthetic construct
  misc_feature 1..23: note = Description of Artificial Sequence: Synthetic oligonucleotide
  source 1..23: mol_type = other DNA
ttggtagggggtgggagggatgg

SEQ ID NO: 204 - DNA, 23 bp, synthetic construct
  misc_feature 1..23: note = Description of Artificial Sequence: Synthetic oligonucleotide
  source 1..23: mol_type = other DNA
gggaaggggagcttggcaggtgg

SEQ ID NO: 205 - DNA, 23 bp, synthetic construct
  misc_feature 1..23: note = Description of Artificial Sequence: Synthetic oligonucleotide
  source 1..23: mol_type = other DNA
ggtagtgagagcttggcaggtgg

SEQ ID NO: 206 - DNA, 23 bp, synthetic construct
  misc_feature 1..23: note = Description of Artificial Sequence: Synthetic oligonucleotide
  source 1..23: mol_type = other DNA
ctccctcccaggatcctcccagg

SEQ ID NO: 207 - DNA, 23 bp, synthetic construct
  misc_feature 1..23: note = Description of Artificial Sequence: Synthetic oligonucleotide
  source 1..23: mol_type = other DNA
gtccatggcaggatcctctcagg

SEQ ID NO: 208 - DNA, 23 bp, synthetic construct
  misc_feature 1..23: note = Description of Artificial Sequence: Synthetic oligonucleotide
  source 1..23: mol_type = other DNA
ctcccccccagtatcctctcagg

SEQ ID NO: 209 - DNA, 108 bp, Homo sapiens
  source 1..108: mol_type = unassigned DNA
ccaatctatgacatcaattattatacatcggagccctgccaaaaaatcaatgtgaagcaaatcgcagcccgcctcctgcctccgctctactcactggtgttcatcttt

SEQ ID NO: 210 - DNA, 138 bp, Homo sapiens
  source 1..138: mol_type = unassigned DNA
ccaccctataattctgaacctgcagaagaatctgaacataaaaacaacaattacgaaccaaacctatttaaaactccacaaaggaaaccatcttataatcagctggcttcaactccaataatattcaaagagcaaggg

SEQ ID NO: 211 - DNA, 49 bp, Homo sapiens
  source 1..49: mol_type = unassigned DNA
ggccgggaatcaagagtcacccagagacagtgaccaaccatccctgttt

SEQ ID NO: 212 - DNA, 77 bp, synthetic construct
  misc_feature 1..77: note = Description of Artificial Sequence: Synthetic oligonucleotide
  source 1..77: mol_type = other DNA
ggccgggaatcaagagtcacccagtgaccaaccatccctgtaagcaaaccttagaggttctggcaaggagagagatg

SEQ ID NO: 213 - DNA, 27 bp, synthetic construct
  misc_feature 1..27: note = Description of Artificial Sequence: Synthetic oligonucleotide
  source 1..27: mol_type = other DNA
ggccgggaatcaagagtcacccaggaa

SEQ ID NO: 214 - DNA, 79 bp, synthetic construct
  misc_feature 1..79: note = Description of Artificial Sequence: Synthetic oligonucleotide
  source 1..79: mol_type = other DNA
ggccgggaatcaagagtcacccagacctctctggctccatcgtaagcaaaccttagaggttctggcaaggagagagatg

SEQ ID NO: 215 - DNA, 28 bp, synthetic construct
  misc_feature 1..28: note = Description of Artificial Sequence: Synthetic oligonucleotide
  source 1..28: mol_type = other DNA
ggccgggaatcaagagtcaccctaacag

SEQ ID NO: 216 - DNA, 66 bp, synthetic construct
  misc_feature 1..66: note = Description of Artificial Sequence: Synthetic oligonucleotide
  source 1..66: mol_type = other DNA
ggccgggaatcaagacgctggctccatcgtaagcaaaccttagaggttctggcaaggagagagatg

SEQ ID NO: 217 - DNA, 47 bp, synthetic construct
  misc_feature 1..47: note = Description of Artificial Sequence: Synthetic oligonucleotide
  source 1..47: mol_type = other DNA
ggctccatcgtaagcaaaccttagaggttctggcaaggagagagatg

SEQ ID NO: 218 - DNA, 78 bp, synthetic construct
  misc_feature 1..78: note = Description of Artificial Sequence: Synthetic oligonucleotide
  source 1..78: mol_type = other DNA
ggccgggaatcaagagtcacccagactctctggctccatcgtaagcaaaccttagaggttctggcaaggagagagatg

SEQ ID NO: 219 - DNA, 80 bp, synthetic construct
  misc_feature 1..80: note = Description of Artificial Sequence: Synthetic oligonucleotide
  source 1..80: mol_type = other DNA
ggccgggaatcaagagtcacccagagacagtgaccaaccatcgtaagcaaaccttagaggttctggcaaggagagagatg

SEQ ID NO: 220 - DNA, 46 bp, synthetic construct
  misc_feature 1..46: note = Description of Artificial Sequence: Synthetic oligonucleotide
  source 1..46: mol_type = other DNA
ggtccatcgtaagcaaaccttagaggttctggcaaggagagagatg

SEQ ID NO: 221 - DNA, 69 bp, synthetic construct
  misc_feature 1..69: note = Description of Artificial Sequence: Synthetic oligonucleotide
  source 1..69: mol_type = other DNA
ggccgggaatcaagagtcacccatctccatcgtaagcaaaccttagaggttctggcaaggagagagatg

SEQ ID NO: 222 - DNA, 76 bp, synthetic construct
  misc_feature 1..76: note = Description of Artificial Sequence: Synthetic oligonucleotide
  source 1..76: mol_type = other DNA
ggccgggaatcaagagtcacccagactctggctccatcgtaagcaaaccttagaggttctggcaaggagagagatg

SEQ ID NO: 223 - DNA, 50 bp, synthetic construct
  misc_feature 1..50: note = Description of Artificial Sequence: Synthetic oligonucleotide
  source 1..50: mol_type = other DNA
ggccgggaatcaagagtcacccagagacagtgaccaaccatcccatatca

SEQ ID NO: 224 - DNA, 59 bp, synthetic construct
  misc_feature 1..59: note = Description of Artificial Sequence: Synthetic oligonucleotide
  source 1..59: mol_type = other DNA
ggccgggaatcaagagtcatcgtaagcaaaccttagaggttctggcaaggagagagatg

SEQ ID NO: 225 - DNA, 24 bp, synthetic construct
  misc_feature 1..24: note = Description of Artificial Sequence: Synthetic oligonucleotide
  source 1..24: mol_type = other DNA
aatgaccactacatccttcaaggg

SEQ ID NO: 226 - DNA, 25 bp, synthetic construct
  misc_feature 1..25: note = Description of Artificial Sequence: Synthetic oligonucleotide
  source 1..25: mol_type = other DNA
aatgaccactacatcctttcaaggg

SEQ ID NO: 227 - DNA, 26 bp, synthetic construct
  misc_feature 1..26: note = Description of Artificial Sequence: Synthetic oligonucleotide
  source 1..26: mol_type = other DNA
aatgaccactacatccttttcaaggg

SEQ ID NO: 228 - DNA, 22 bp, synthetic construct
  misc_feature 1..22: note = Description of Artificial Sequence: Synthetic oligonucleotide
  source 1..22: mol_type = other DNA
aatgaccactacatcctaaggg

SEQ ID NO: 229 - DNA, 21 bp, synthetic construct
  misc_feature 1..21: note = Description of Artificial Sequence: Synthetic oligonucleotide
  source 1..21: mol_type = other DNA
aatgaccactacatcctaggg

SEQ ID NO: 230 - DNA, 20 bp, synthetic construct
  misc_feature 1..20: note = Description of Artificial Sequence: Synthetic oligonucleotide
  source 1..20: mol_type = other DNA
aatgaccactacatcctggg

SEQ ID NO: 231 - DNA, 42 bp, Homo sapiens
  source 1..42: mol_type = unassigned DNA
tatgtgcaatgaccactacatcctcaagggcagcaatcggag

SEQ ID NO: 232 - DNA, 45 bp, synthetic construct
  misc_feature 1..45: note = Description of Artificial Sequence: Synthetic oligonucleotide
  source 1..45: mol_type = other DNA
tatgtgcaatgaccactacatcctcctcaagggcagcaatcggag

SEQ ID NO: 233 - DNA, 30 bp, synthetic construct
  misc_feature 1..30: note = Description of Artificial Sequence: Synthetic oligonucleotide
  source 1..30: mol_type = other DNA
tatgtgcaatgaccactacatcaatcggag

SEQ ID NO: 234 - DNA, 33 bp, synthetic construct
  misc_feature 1..33: note = Description of Artificial Sequence: Synthetic oligonucleotide
  source 1..33: mol_type = other DNA
tatgtgcaatgaccactacatcagcaatcggag

SEQ ID NO: 235 - DNA, 34 bp, synthetic construct
  misc_feature 1..34: note = Description of Artificial Sequence: Synthetic oligonucleotide
  source 1..34: mol_type = other DNA
tatgtgcaatgaccactacatccagcaatcggag

SEQ ID NO: 236 - DNA, 10 bp, synthetic construct
  misc_feature 1..10: note = Description of Artificial Sequence: Synthetic oligonucleotide
  source 1..10: mol_type = other DNA
cagcaatcgg

SEQ ID NO: 237 - DNA, 41 bp, synthetic construct
  misc_feature 1..41: note = Description of Artificial Sequence: Synthetic oligonucleotide
  source 1..41: mol_type = other DNA
tatgtgcaatgaccactacatccttcaagggcagcaatcgg

SEQ ID NO: 238 - DNA, 41 bp, synthetic construct
  misc_feature 1..41: note = Description of Artificial Sequence: Synthetic oligonucleotide
  source 1..41: mol_type = other DNA
tatgtgcaatgaccactacatcctccaagggcagcaatcgg

SEQ ID NO: 239 - DNA, 41 bp, synthetic construct
  misc_feature 1..41: note = Description of Artificial Sequence: Synthetic oligonucleotide
  source 1..41: mol_type = other DNA
tatgtgcaatgaccactacatcctbcaagggcagcaatcgg

SEQ ID NO: 240 - DNA, 34 bp, synthetic construct
  misc_feature 1..34: note = Description of Artificial Sequence: Synthetic oligonucleotide
  source 1..34: mol_type = other DNA
tatgtgcaatgaccactacattggcagcaatcgg

SEQ ID NO: 241 - DNA, 21 bp, synthetic construct
  misc_feature 1..21: note = Description of Artificial Sequence: Synthetic oligonucleotide
  source 1..21: mol_type = other DNA
tatgtgcaatgaccactacat

SEQ ID NO: 242 - DNA, 53 bp, Homo sapiens
  source 1..53: mol_type = unassigned DNA
tcatacagatgatgtctcatcatcagaggagcgagaaggtaaagtcaaaatca

SEQ ID NO: 243 - DNA, 32 bp, synthetic construct
  misc_feature 1..32: note = Description of Artificial Sequence: Synthetic oligonucleotide
  source 1..32: mol_type = other DNA
tcatacagatgatacaggtaaagtcaaaatca

SEQ ID NO: 244 - DNA, 32 bp, synthetic construct
  misc_feature 1..32: note = Description of Artificial Sequence: Synthetic oligonucleotide
  source 1..32: mol_type = other DNA
tcatacaggtgatgaaggtaaagtcaaaatca

SEQ ID NO: 245 - DNA, 50 bp, synthetic construct
  misc_feature 1..50: note = Description of Artificial Sequence: Synthetic oligonucleotide
  source 1..50: mol_type = other DNA
tcatacagatgatgtctcatcatcagagcgagaaggtaaagtcaaaatca

SEQ ID NO: 246 - DNA, 48 bp, synthetic construct
  misc_feature 1..48: note = Description of Artificial Sequence: Synthetic oligonucleotide
  source 1..48: mol_type = other DNA
tcatacagatgatgtctcatcatcagcgagaaggtaaagtcaaaatca

SEQ ID NO: 247 - DNA, 52 bp, synthetic construct
  misc_feature 1..52: note = Description of Artificial Sequence: Synthetic oligonucleotide
  source 1..52: mol_type = other DNA
tcatacagatgatgtctcatcatcagggagcgagaaggtaaagtcaaaatca

SEQ ID NO: 248 - DNA, 41 bp, synthetic construct
  misc_feature 1..41: note = Description of Artificial Sequence: Synthetic oligonucleotide
  source 1..41: mol_type = other DNA
tcatacagatgatgtctcgcgagaaggtaaagtcaaaatca

SEQ ID NO: 249 - DNA, 32 bp, synthetic construct
  misc_feature 1..32: note = Description of Artificial Sequence: Synthetic oligonucleotide
  source 1..32: mol_type = other DNA
tcatacagatgatgaaggtaaagtcaaaatca

SEQ ID NO: 250 - DNA, 29 bp, synthetic construct
  misc_feature 1..29: note = Description of Artificial Sequence: Synthetic oligonucleotide
  source 1..29: mol_type = other DNA
tcatacagatgaaggtaaagtcaaaatca

SEQ ID NO: 251 - DNA, 42 bp, synthetic construct
  misc_feature 1..42: note = Description of Artificial Sequence: Synthetic oligonucleotide
  source 1..42: mol_type = other DNA
tcatacagatgatgtctacagatgaaggtaaagtcaaaatca

SEQ ID NO: 252 - DNA, 51 bp, synthetic construct
  misc_feature 1..51: note = Description of Artificial Sequence: Synthetic oligonucleotide
  source 1..51: mol_type = other DNA
tcatacagatgatgtctcatcatcaggagcgagaaggtaaagtcaaaatca

SEQ ID NO: 253 - DNA, 34 bp, synthetic construct
  misc_feature 1..34: note = Description of Artificial Sequence: Synthetic oligonucleotide
  source 1..34: mol_type = other DNA
gtcatcctcatcctgataaactgcaaaaggctga

SEQ ID NO: 254 - DNA, 606 bp, Homo sapiens
  source 1..606: mol_type = unassigned DNA
gctggtgtctgggttctgtgccccttccccaccccagcccaccccaggtgtcctgtccattctcaggctggtcacatgggtggtcctagggtgtcccatgagagatgcaaagcgcctgaattttctgactcttcccatcagaccccccaaagacacatgtgacccaccaccccatctctgaccatgaggccaccctgaggtgctgggccctgggcttctaccctgcggagatcacactgacctggcagcgggatggcgaggaccaaactcaggacaccgagcttgtggagaccagaccagcaggagatagaaccttccagaagtgggcagctgtggtggtgccttctggagaagagcagagatacacatgccatgtacagcatgaggggctgccgaagcccctcaccctgagatggggtaaggagggggatgaggggtcatatctgttcatatctgttctcagggaaagcaggagcccttctggagcccttcagcagggtcagggcccctcatcttcccctcctttcccagagccatcttcccagtccaccatccccatcgtgggcattgttgctggcctggctgtcctagcagttgtggtcatcg

SEQ ID NO: 255 - DNA, 26 bp, Homo sapiens
  source 1..26: mol_type = unassigned DNA
actaccacagctccttctctgagtgg

SEQ ID NO: 256 - DNA, 23 bp, synthetic construct
  misc_feature 1..23: note = Description of Artificial Sequence: Synthetic oligonucleotide
  source 1..23: mol_type = other DNA
actaccacagctcctctgagtgg

SEQ ID NO: 257 - DNA, 23 bp, Homo sapiens
  source 1..23: mol_type = unassigned DNA
gtagttggagctggcggcgtagg

SEQ ID NO: 258 - DNA, 23 bp, synthetic construct
  misc_feature 1..23: note = Description of Artificial Sequence: Synthetic oligonucleotide
  source 1..23: mol_type = other DNA
gtagttggagctagcggcgtagg

SEQ ID NO: 259 - DNA, 23 bp, Homo sapiens
  source 1..23: mol_type = unassigned DNA
gtagttggagctggtggcgtagg

SEQ ID NO: 260 - DNA, 23 bp, synthetic construct
  misc_feature 1..23: note = Description of Artificial Sequence: Synthetic oligonucleotide
  source 1..23: mol_type = other DNA
gtagttggagctagtggcgtagg

SEQ ID NO: 261 - DNA, 28 bp, synthetic construct
  misc_feature 1..28: note = Description of Artificial Sequence: Synthetic oligonucleotide
  source 1..28: mol_type = other DNA
ccatacattaaagatagtcatcttgggg

SEQ ID NO: 262 - DNA, 60 bp, Homo sapiens
  source 1..60: mol_type = unassigned DNA
ccatacagtcagtatcaattctggaagaatttccagacattaaagatagtcatcttgggg

SEQ ID NO: 263 - DNA, 55 bp, Homo sapiens
  source 1..55: mol_type = unassigned DNA
ccatacagtcagtatcaattctggaagaatttccagacattaaagatagtcatct

SEQ ID NO: 264 - DNA, 23 bp, synthetic construct
  misc_feature 1..23: note = Description of Artificial Sequence: Synthetic oligonucleotide
  source 1..23: mol_type = other DNA
ccatacattaaagatagtcatct

SEQ ID NO: 265 - DNA, 23 bp, Homo sapiens
  source 1..23: mol_type = unassigned DNA
agatgactatctttaatgtctgg

SEQ ID NO: 266 - DNA, 23 bp, Homo sapiens
  source 1..23: mol_type = unassigned DNA
agatgactatctttaatgtatgg

SEQ ID NO: 267 - DNA, 23 bp, synthetic construct
  misc_feature 1..23: note = Description of Artificial Sequence: Synthetic oligonucleotide
  source 1..23: mol_type = other DNA
gtagttggagctgatggcgtagg

SEQ ID NO: 268 - DNA, 23 bp, synthetic construct
  misc_feature 1..23: note = Description of Artificial Sequence: Synthetic oligonucleotide
  source 1..23: mol_type = other DNA
gtagttggagctggtagcgtagg

SEQ ID NO: 269 - DNA, 23 bp, synthetic construct
  misc_feature 1..23: note = Description of Artificial Sequence: Synthetic oligonucleotide
  source 1..23: mol_type = other DNA
gtagttggagctggtgacgtagg

SEQ ID NO: 270 - DNA, 23 bp, synthetic construct
  misc_feature 1..23: note = Description of Artificial Sequence: Synthetic oligonucleotide
  source 1..23: mol_type = other DNA
gtagttggagctaatggcgtagg

SEQ ID NO: 271 - DNA, 23 bp, synthetic construct
  misc_feature 1..23: note = Description of Artificial Sequence: Synthetic oligonucleotide
  source 1..23: mol_type = other DNA
gtagttggagctagtagcgtagg

SEQ ID NO: 272 - DNA, 23 bp, synthetic construct
  misc_feature 1..23: note = Description of Artificial Sequence: Synthetic oligonucleotide
  source 1..23: mol_type = other DNA
gtagttggagctagtgacgtagg

SEQ ID NO: 273 - DNA, 23 bp, Homo sapiens
  source 1..23: mol_type = unassigned DNA
caaatgaatgatgcacatcatgg

SEQ ID NO: 274 - DNA, 23 bp, synthetic construct
  misc_feature 1..23: note = Description of Artificial Sequence: Synthetic oligonucleotide
  source 1..23: mol_type = other DNA
caaatgaatgatgcacgtcatgg

SEQ ID NO: 275 - DNA, 23 bp, synthetic construct
  misc_feature 1..23: note = Description of Artificial Sequence: Synthetic oligonucleotide
  source 1..23: mol_type = other DNA
caaatgaatgatgcatatcatgg

SEQ ID NO: 276 - DNA, 23 bp, synthetic construct
  misc_feature 1..23: note = Description of Artificial Sequence: Synthetic oligonucleotide
  source 1..23: mol_type = other DNA
caaatgaatgatgcgcatcatgg

SEQ ID NO: 277 - DNA, 23 bp, synthetic construct
  misc_feature 1..23: note = Description of Artificial Sequence: Synthetic oligonucleotide
  source 1..23: mol_type = other DNA
caaatgaatgatgtacatcatgg

SEQ ID NO: 278 - DNA, 23 bp, synthetic construct
  misc_feature 1..23: note = Description of Artificial Sequence: Synthetic oligonucleotide
  source 1..23: mol_type = other DNA
caaatgaatggtgcacatcatgg

SEQ ID NO: 279 - DNA, 23 bp, synthetic construct
  misc_feature 1..23: note = Description of Artificial Sequence: Synthetic oligonucleotide
  source 1..23: mol_type = other DNA
caaatgagtgatgcacatcatgg

SEQ ID NO: 280 - DNA, 23 bp, synthetic construct
  misc_feature 1..23: note = Description of Artificial Sequence: Synthetic oligonucleotide
  source 1..23: mol_type = other DNA
caaaagaatgatgcacatcatgg

SEQ ID NO: 281 - DNA, 23 bp, synthetic construct
  misc_feature 1..23: note = Description of Artificial Sequence: Synthetic oligonucleotide
  source 1..23: mol_type = other DNA
cgaatgaatgatgcacatcatgg

SEQ ID NO: 282 - DNA, 23 bp, synthetic construct
  misc_feature 1..23: note = Description of Artificial Sequence: Synthetic oligonucleotide
  source 1..23: mol_type = other DNA
caaatgaatgatgcatgtcatgg

SEQ ID NO: 283 - DNA, 23 bp, synthetic construct
  misc_feature 1..23: note = Description of Artificial Sequence: Synthetic oligonucleotide
  source 1..23: mol_type = other DNA
caaatgaatgatgcgcgtcatgg

SEQ ID NO: 284 - DNA, 23 bp, synthetic construct
  misc_feature 1..23: note = Description of Artificial Sequence: Synthetic oligonucleotide
  source 1..23: mol_type = other DNA
caaatgaatgatgtacgtcatgg

SEQ ID NO: 285 - DNA, 23 bp, synthetic construct
  misc_feature 1..23: note = Description of Artificial Sequence: Synthetic oligonucleotide
  source 1..23: mol_type = other DNA
caaatgaatggtgcacgtcatgg

SEQ ID NO: 286 - DNA, 23 bp, synthetic construct
  misc_feature 1..23: note = Description of Artificial Sequence: Synthetic oligonucleotide
  source 1..23: mol_type = other DNA
caaatgagtgatgcacgtcatgg

SEQ ID NO: 287 - DNA, 23 bp, synthetic construct
  misc_feature 1..23: note = Description of Artificial Sequence: Synthetic oligonucleotide
  source 1..23: mol_type = other DNA
caaaagaatgatgcacgtcatgg

SEQ ID NO: 288 - DNA, 23 bp, synthetic construct
  misc_feature 1..23: note = Description of Artificial Sequence: Synthetic oligonucleotide
  source 1..23: mol_type = other DNA
cgaatgaatgatgcacgtcatgg

SEQ ID NO: 289 - DNA, 23 bp, Homo sapiens
  source 1..23: mol_type = unassigned DNA
atcataggtcgtcatgcttatgg

SEQ ID NO: 290 - DNA, 23 bp, synthetic construct
  misc_feature 1..23: note = Description of Artificial Sequence: Synthetic oligonucleotide
  source 1..23: mol_type = other DNA
atcataggttgtcatgcttatgg

SEQ ID NO: 291 - DNA, 23 bp, Homo sapiens
  source 1..23: mol_type = unassigned DNA
atcataggtcgtcctgcttatgg

SEQ ID NO: 292 - DNA, 23 bp, synthetic construct
  misc_feature 1..23: note = Description of Artificial Sequence: Synthetic oligonucleotide
  source 1..23: mol_type = other DNA
atcataggttgtcctgcttatgg

SEQ ID NO: 293 - DNA, 23 bp, Homo sapiens
  source 1..23: mol_type = unassigned DNA
ctggacaagaagagtacagtgcc

SEQ ID NO: 294 - DNA, 23 bp, synthetic construct
  misc_feature 1..23: note = Description of Artificial Sequence: Synthetic oligonucleotide
  source 1..23: mol_type = other DNA
ctggaaaagaagagtacagtgcc

SEQ ID NO: 295 - DNA, 23 bp, Homo sapiens
  source 1..23: mol_type = unassigned DNA
actccatcgagatttcactgtag

SEQ ID NO: 296 - DNA, 23 bp, synthetic construct
  misc_feature 1..23: note = Description of Artificial Sequence: Synthetic oligonucleotide
  source 1..23: mol_type = other DNA
actccatcgagatttctctgtag

SEQ ID NO: 297 - DNA, 34 bp, synthetic construct
  misc_feature 1..34: note = Description of Artificial Sequence: Synthetic oligonucleotide
  source 1..34: mol_type = other DNA
acttccaggcaaccccaaggggacctcatgcagg

SEQ ID NO: 298 - DNA, 35 bp, synthetic construct
  misc_feature 1..35: note = Description of Artificial Sequence: Synthetic oligonucleotide
  source 1..35: mol_type = other DNA
acttccaggcgaaccccaaggggacctcatgcagg

SEQ ID NO: 299 - DNA, 13 bp, synthetic construct
  misc_feature 1..13: note = Description of Artificial Sequence: Synthetic oligonucleotide
  source 1..13: mol_type = other DNA
acttccaggctcc

SEQ ID NO: 300 - DNA, 13 bp, synthetic construct
  misc_feature 1..13: note = Description of Artificial Sequence: Synthetic oligonucleotide
  source 1..13: mol_type = other DNA
acttccaggctcc

SEQ ID NO: 301 - DNA, 32 bp, synthetic construct
  misc_feature 1..32: note = Description of Artificial Sequence: Synthetic oligonucleotide
  source 1..32: mol_type = other DNA
acttccaggctcccaaggggacctcatgcagg

SEQ ID NO: 302 - DNA, 41 bp, synthetic construct
  misc_feature 1..41: note = Description of Artificial Sequence: Synthetic oligonucleotide
  source 1..41: mol_type = other DNA
acttccaggctccttaggaggcgaaccccaaggggacctca

SEQ ID NO: 303 - DNA, 25 bp, synthetic construct
  misc_feature 1..25: note = Description of Artificial Sequence: Synthetic oligonucleotide
  source 1..25: mol_type = other DNA
acttccaggctccacctcatgcagg

SEQ ID NO: 304 - DNA, 32 bp, synthetic construct
  misc_feature 1..32: note = Description of Artificial Sequence: Synthetic oligonucleotide
  source 1..32: mol_type = other DNA
acttccaggctccacccccaagggacctcatg

SEQ ID NO: 305 - DNA, 35 bp, synthetic construct
  misc_feature 1..35: note = Description of Artificial Sequence: Synthetic oligonucleotide
  source 1..35: mol_type = other DNA
acttccaggctccacccaaggggacctcatgcagg

SEQ ID NO: 306 - DNA, 36 bp, synthetic construct; identical to SEQ ID NO: 137
  misc_feature 1..36: note = Description of Artificial Sequence: Synthetic oligonucleotide
  source 1..36: mol_type = other DNA

SEQ ID NO: 307 - DNA, 38 bp, synthetic construct
  misc_feature 1..38: note = Description of Artificial Sequence: Synthetic oligonucleotide
  source 1..38: mol_type = other DNA
acttccaggctccacccacccaaggggacctcatgcag

SEQ ID NO: 308 - DNA, 38 bp, synthetic construct
  misc_feature 1..38: note = Description of Artificial Sequence: Synthetic oligonucleotide
  source 1..38: mol_type = other DNA
acttccaggctccaccccacccaaggggacctcatgca

SEQ ID NO: 309 - DNA, 40 bp, synthetic construct
  misc_feature 1..40: note = Description of Artificial Sequence: Synthetic oligonucleotide
  source 1..40: mol_type = other DNA
acttccaggctccacccaccccaaggggacctcatgcagg

SEQ ID NO: 310 - DNA, 45 bp, synthetic construct
  misc_feature 1..45: note = Description of Artificial Sequence: Synthetic oligonucleotide
  source 1..45: mol_type = other DNA
acttccaggctccacccggcgaaccccaaggggacctcatgcagg

SEQ ID NO: 311 - DNA, 34 bp, synthetic construct
  misc_feature 1..34: note = Description of Artificial Sequence: Synthetic oligonucleotide
  source 1..34: mol_type = other DNA
acttccaggctccaccctggggacctcatgcagg

SEQ ID NO: 312 - DNA, 42 bp, synthetic construct
  misc_feature 1..42: note = Description of Artificial Sequence: Synthetic oligonucleotide
  source 1..42: mol_type = other DNA
acttccaggctccacccgaaccccaaggggacctcatgcagg

SEQ ID NO: 313 - DNA, 31 bp, synthetic construct
  misc_feature 1..31: note = Description of Artificial Sequence: Synthetic oligonucleotide
  source 1..31: mol_type = other DNA
acttccaggctccacccgaacctcatgcagg

SEQ ID NO: 314 - DNA, 35 bp, synthetic construct
  misc_feature 1..35: note = Description of Artificial Sequence: Synthetic oligonucleotide
  source 1..35: mol_type = other DNA
acttccaggctccacccgaggggacctcatgcagg

SEQ ID NO: 315 - DNA, 36 bp, synthetic construct; identical to SEQ ID NO: 137
  misc_feature 1..36: note = Description of Artificial Sequence: Synthetic oligonucleotide
  source 1..36: mol_type = other DNA

SEQ ID NO: 316 - DNA, 42 bp, synthetic construct; identical to SEQ ID NO: 312
  misc_feature 1..42: note = Description of Artificial Sequence: Synthetic oligonucleotide
  source 1..42: mol_type = other DNA

SEQ ID NO: 317 - DNA, 43 bp, synthetic construct
  misc_feature 1..43: note = Description of Artificial Sequence: Synthetic oligonucleotide
  source 1..43: mol_type = other DNA
acttccaggctccacccgagaccccaaggggacctcatgcagg

SEQ ID NO: 318 - DNA, 48 bp, synthetic construct
  misc_feature 1..48: note = Description of Artificial Sequence: Synthetic oligonucleotide
  source 1..48: mol_type = other DNA
acttccaggctccacccgagggcgaaccccaaggggacctcatgcagg

SEQ ID NO: 319 - DNA, 30 bp, synthetic construct; identical to SEQ ID NO: 136
  misc_feature 1..30: note = Description of Artificial Sequence: Synthetic oligonucleotide
  source 1..30: mol_type = other DNA

SEQ ID NO: 320 - DNA, 42 bp, synthetic construct
  misc_feature 1..42: note = Description of Artificial Sequence: Synthetic oligonucleotide
  source 1..42: mol_type = other DNA
acttccaggctccacccgacccccaaggggacctcatgcagg

SEQ ID NO: 321 - DNA, 42 bp, synthetic construct
  misc_feature 1..42: note = Description of Artificial Sequence: Synthetic oligonucleotide
  source 1..42: mol_type = other DNA
acttccaggctccacccgacgaagggccccaaggggacctca

SEQ ID NO: 322 - DNA, 45 bp, synthetic construct; identical to SEQ ID NO: 141
  misc_feature 1..45: note = Description of Artificial Sequence: Synthetic oligonucleotide
  source 1..45: mol_type = other DNA

SEQ ID NO: 323 - DNA, 48 bp, synthetic construct
  misc_feature 1..48: note = Description of Artificial Sequence: Synthetic oligonucleotide
  source 1..48: mol_type = other DNA
acttccaggctccacccgacggcgaaccccaaggggacctcatgcagg

SEQ ID NO: 324 - DNA, 51 bp, synthetic construct
  misc_feature 1..51: note = Description of Artificial Sequence: Synthetic oligonucleotide
  source 1..51: mol_type = other DNA
acttccaggctccacccgacgtgcttgagggcgaaccccaaggggacctca

SEQ ID NO: 325 - DNA, 47 bp, synthetic construct; identical to SEQ ID NO: 140
  misc_feature 1..47: note = Description of Artificial Sequence: Synthetic oligonucleotide
  source 1..47: mol_type = other DNA

SEQ ID NO: 326 - DNA, 49 bp, synthetic construct
  misc_feature 1..49: note = Description of Artificial Sequence: Synthetic oligonucleotide
  source 1..49: mol_type = other DNA
acttccaggctccacccgacttggcgaaccccaaggggacctcatgcag

SEQ ID NO: 327 - DNA, 51 bp, synthetic construct
  misc_feature 1..51: note = Description of Artificial Sequence: Synthetic oligonucleotide
  source 1..51: mol_type = other DNA
acttccaggctccacccgacttgcagggcgaaccccaaggggacctcatgc

SEQ ID NO: 328 - DNA, 53 bp, synthetic construct
  misc_feature 1..53: note = Description of Artificial Sequence: Synthetic oligonucleotide
  source 1..53: mol_type = other DNA
acttccaggctccacccgacttggagggcgaaccccaaggggacctcatgcag

SEQ ID NO: 329 - DNA, 53 bp, synthetic construct
  misc_feature 1..53: note = Description of Artificial Sequence: Synthetic oligonucleotide
  source 1..53: mol_type = other DNA
acttccaggctccacccgactttggagggcgaaccccaaggggacctcatgca

SEQ ID NO: 330 - DNA, 53 bp, synthetic construct
  misc_feature 1..53: note = Description of Artificial Sequence: Synthetic oligonucleotide
  source 1..53: mol_type = other DNA
acttccaggctccacccgactgttggagggcgaaccccaaggggacctcatgc

SEQ ID NO: 331 - DNA, 502 bp, synthetic construct
  misc_feature 1..502: note = Description of Artificial Sequence: Synthetic oligonucleotide
  misc_difference 26..480: note = misc_feature - a, c, t, g, unknown or other
  source 1..502: mol_type = other DNA
acttccaggctccacccgactggagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcgaaccccaaggggacctcc

SEQ ID NO: 332 - DNA, 72 bp, Homo sapiens
  source 1..72: mol_type = unassigned DNA
agctctccctcccaggatcctctctggctccatcgtaagcaaaccttagaggttctggcaaggagagagatg